(19) United States
(12) Patent Application Publication
(10) Pub. No.: US 2004/0002105 A1
(43) Pub. Date: Jan. 1, 2004

(54) Methods of identifying genes for the manipulation of triterpene saponins

(75) Inventors: Richard A. Dixon, Ardmore, OK (US); Lahoucine Achnine, Ardmore, OK (US); Hideyuki Suzuki, Kisarazu-shi (JP); Xian-Zhi He, Ardmore, OK (US); Liangjiang Wang, Ardmore, OK (US)

Correspondence Address: Robert E. Hanson, Fulbright & Jaworski, L.L.P., Suite 2400, 600 Congress Avenue, Austin, TX, 78701, US

(73) Assignee: The Samuel Roberts Noble Foundation

(21) Appl. No.: 10/429,949

(22) Filed: May 5, 2003

Related U.S. Application Data
(60) Provisional application No. 60/380,159, filed on May 4, 2002.

(51) Int. Cl. C12Q 1/68; G01N 33/53; G01N 33/567; A01H 1/00; C12N 15/82

(52) U.S. Cl. 435/6; 435/7.2; 800/278

(57) ABSTRACT

The invention provides methods for the isolation of plant genes and their regulatory sequences involved in the biosynthesis of triterpene saponins. Also provided by the invention are genes involved in the biosynthesis of triterpenes, including squalene synthase, squalene epoxidase and β-amyrin synthase from Medicago truncatula. The identification of triterpene biosynthesis genes allows genetic modification of the content and composition of triterpene saponins in plants for crop improvement and the development of drugs, nutriceuticals and functional foods.

### BACKGROUND OF THE INVENTION

This application claims the priority of U.S. provisional patent application serial No. 60/380,159, filed May 4, 2002, the entire disclosure of which is specifically incorporated herein by reference.

### FIELD OF THE INVENTION

The present invention generally relates to molecular biology. More specifically, the invention relates to methods for the isolation of genes in the triterpene biosynthetic pathway and the genes isolated by these methods.

### DESCRIPTION OF THE RELATED ART

Triterpene glycoside saponins are attracting increasing interest in view of their multiple biological activities. These both positively and negatively impact plant traits. Thus, whereas some saponins display allelopathic (Waller et al., 1993), anti-microbial (Nagata et al., 1985; Papadopoulou et al., 1999; Osbourn, 1996), and anti-insect (Pedersen et al., 1976; Tava and Odorati, 1997) activity, they can also be toxic to monogastric animals, act as anti-palatability factors, or negatively impact forage digestibility in ruminants (Cheeke, 1976; Oleszek, 1997). Other saponins have potentially useful pharmacological activities, including anticholesterolemic (Cheeke, 1976), anti-cancer (Haridas et al., 2001; Park et al., 2001), adjuvant (Behboudi et al., 1999; Marciani et al., 2000), and hemolytic (Jones and Elliott, 1969) activity. Triterpene saponins therefore have a wide variety of potential uses in medicine, either as drugs, nutriceuticals, or components of functional foods. In addition, they could be manipulated in crop plants to improve disease or pest resistance, or in some cases reduced in forage crops to improve palatability. Despite the interest in facilitating or inhibiting production of triterpene saponins for crop improvement or development of pharmacological agents, most of the steps in their biosynthesis remain uncharacterized at the molecular level. Thus, discovery of genes involved in triterpene saponin biosynthesis is necessary to facilitate the engineering of triterpene saponin levels in transgenic plants.

The model legume Medicago truncatula is a suitable species for a functional genomics approach to triterpene saponin biosynthesis in view of the availability of extensive EST resources (Bell et al., 2001) and the interesting and complex saponin profile of this species (Huhman and Sumner, 2002). Metabolic profiling of M. truncatula roots using reverse-phase HPLC and electrospray ionization mass spectrometry showed the presence of a more complex mixture of triterpenes than found in the closely related and previously well studied species alfalfa (Medicago sativa) (Tava et al., 1993; Massiot et al., 1988; Oleszek and Jurzysta, 1990; Oleszek et al., 1992). Five different triterpene aglycones, soyasapogenol B, soyasapogenol E, medicagenic acid, hederagenin and bayogenin were found to be the core of the thirty seven M. truncatula saponins identified (Huhman and Sumner, 2002). These aglycones are most likely all derived from β-amyrin, the initial product of cyclization of 2,3-oxidosqualene.

The first committed step in triterpene biosynthesis in Medicago is catalyzed by a specific oxidosqualene cyclase (OCS), β-amyrin synthase (β-AS). In higher plants, oxidosqualene is a precursor common to the biosynthesis of both steroids and triterpenoids (Abe and Prestwich, 1993). In sterol biosynthesis in animals and fungi, the cyclization of 2,3-oxidosqualene leads to the formation of lanosterol, whereas cycloartenol is the first cyclized sterol precursor in plants. β-AS has been functionally characterized from Panax ginseng (Kushiro et al., 1998), pea (Morita et al., 2000) and Arabidopsis thaliana (Husselstein-Muller et al., 2001), and is closely related to plant cycloartenol synthase, which has also been cloned and functionally characterized (Corey et al., 1993; Hayashi et al., 2000). Surprisingly, a recently characterized monocot β-AS from oat is phylogenetically distinct from dicot β-AS enzymes (Haralampidis et al., 2001). β-AS may produce one or more products from the cyclization of 2,3-oxidosqualene, depending on the plant source (Abe and Prestwich, 1993; Kushiro et al., 1998; Kushiro et al., 2000; Husselstein-Muller et al., 2001). Thus, it is not clear from sequence information alone whether a particular oxidosqualene cyclase will be a β-amyrin synthase or, if so, whether it will make β-amyrin alone or a mixture of related triterpenes.

The two enzymes preceding OSC, namely squalene synthase (SS) and squalene epoxidase (SE), have been characterized in mammals and yeast (Jandrositz et al., 1991; Laden et al., 2000; Lee et al., 2000; Pandit et al., 2000). SS has been functionally characterized from Arabidopsis (Nakashima et al., 1995; Kribii et al., 1997). Mammalian SE plays a pivotal role in cholesterol biosynthesis, and the enzyme is expressed at low levels in most tissues (Yamaoto and Bloch, 1970; Ono and Bloch, 1975). Detailed enzymological characterization of human SE has been reported (Laden et al, 2000). In yeast, the squalene epoxidase Erg1p exhibits dual localization in the endoplasmic reticulum and in lipid particles (Leber et al, 1998). However, although plant SE genes have been annotated based upon sequence similarity to the mammalian and yeast enzymes (Schäfer et al., 1999), plant SE has not been functionally characterized. SE is membrane associated, requires NADPH cytochrome P450 reductase and, in mammals, additional soluble protein factors for its activity (Laden et al., 2000; Shibata et al., 2001). It has not been known whether additional proteins are required for the functional expression of SE in plants, or whether specific forms of SS and SE might be differentially associated with sterol and triterpene biosynthesis in plants.

The characterization of genes involved in the biosynthesis of triterpenes has been difficult. Extraction and quantitation of the multiple M. truncatula triterpene saponins is not trivial and is therefore not the best assay method for determining expression of the triterpene pathway (Huhman and Sumner, 2002). Further, triterpenes are often not expressed at high basal levels. Previous studies have shown effects of sucrose and mineral nutrients on saponin production in plant cell suspension cultures, but these effects were neither large nor rapid (Fulcheri et al., 1998). Stimulation of the growth and the triterpenoid saponin accumulation of Saponaria officinalis cell and Gypsophila paniculata root suspension cultures by improvement of the mineral composition of the media have been attempted (Akalezi et al., 1999). The association of methyl jasmonate and the production of the triterpenes oleanolic acid and ursolic acid in Sculellaria baicalens has been mentioned (Yoon et al., 2000). However, methyl jasmonate was found to be a weak inducer of triterpene biosynthesis relative to yeast elicitor and it is not known if Medicago cultures produce oleanolic and ursolic acids. Sculellaria baicalens is further not a legume and thus no conclusion can be drawn regarding this discussion.

However, what has been lacking is a system for the induction of high-level expression of triterpene saponins in legumes. Development of such a system would represent an important advance and would potentially allow the implementation of high-throughput techniques for the isolation of the genes involved in the triterpene biosynthetic pathway.

### SUMMARY OF THE INVENTION

In one aspect, the invention provides a method of identifying a triterpene biosynthesis gene comprising: (a) obtaining a cell from a target legume species; (b) contacting said cell with methyl jasmonate; and (c) identifying a coding sequence which is specifically upregulated in the cell following the contacting with methyl jasmonate to identify a triterpene biosynthesis gene. The method may further comprise screening a polypeptide encoded by the coding sequence for the ability to catalyze a step in triterpene biosynthesis. In one embodiment of the invention, the target legume is selected from the group consisting of soybean, alfalfa, Medicago truncatula, peanuts, beans, peas, lentils, Lotus japonicus, chickpea, cowpea, lupin, vetch, Sophora species, Acacia species, licorice and clover. The cell may be grown in, for example, a tissue culture, including a suspension culture.

In one embodiment of the invention, the step of obtaining a cell is further defined as comprising obtaining a population of cells from the target legume. The cell may be obtained from a plant and may also be obtained from a tissue culture, including a suspension culture. In a further embodiment of the invention, the step of identifying a coding sequence is further defined as comprising identifying a plurality of coding sequences specifically upregulated in said cell relative to the corresponding coding sequences in one or more other cells which have not been contacted with methyl jasmonate. In yet another embodiment of the invention, the step of identifying a coding sequence comprises obtaining an RNA transcribed by the coding sequence and/or a cDNA derived therefrom.

In certain aspects of the invention, the method of identifying a triterpene biosynthesis gene may further comprise the steps of: (a) labeling said RNA and/or cDNA; and (b) hybridizing the labeled RNA or cDNA to an array comprising a plurality of coding sequences from the target legume. The method may further comprise preparing an array comprising the RNA transcripts or cDNAs thereof arranged on a support material. In certain embodiments of the invention, identifying a coding sequence further comprises selecting a coding sequence having homology to a cytochrome P450, glycosyltransferase, squalene synthase, squalene epoxidase and/or β-amyrin synthase gene.

In further embodiments of the invention, identifying a coding sequence comprises use of subtractive hybridization, nucleic acid sequencing, RT-PCR, and/or differential display. In other embodiments, screening comprises transforming a host cell with the coding sequence and determining the ability of the host cell to catalyze a step in triterpene biosynthesis. This may additionally comprise contacting the host cell with a substrate of said step in triterpene biosynthesis including, but not necessarily limited to farnesyl diphosphate, squalene, oxidosqualene, β-amyrin, bayogenin, hederagenin, medicagenic acid, soyasapogenol B and soyasapogenol E. In the method, the host cell may be any type of cell, including a yeast, bacterial or plant cell. Where the cell is a plant cell, the method may further comprise regenerating a plant from the plant cell.

In still further embodiments of the invention, a polypeptide is provided encoded by a nucleic acid sequence of any one of SEQ ID NOs:18-31. Also provided are nucleic acids encoding these polypeptides. In one embodiments of the invention, the nucleic acid sequence has a sequence selected from SEQ ID NOs:18-31. In still other embodiments, transformation constructs, including expression cassettes, are provided comprising a nucleic acid encoding a polypeptide encoded by the nucleic acid sequence of any of SEQ ID NOs:18-31 operably linked to a heterologous promoter. Methods are also provided for modification of saponin biosynthesis, including increasing or decreasing triterpenes and/or intermediates in the triterpene biosynthetic pathway, in a plant comprising introducing such constructs, either directly or by plant breeding methods, into a plant.

### BRIEF DESCRIPTION OF THE DRAWINGS

FIGS. 1A-D. DNA gel blot analysis of triterpene pathway genes in M. truncatula. Genomic DNA was cut with the enzymes shown (B=BamHI, E=EcoRI, S=SalI, X=XbaI), fragments resolved by agarose gel electrophoresis, and blots probed with cDNAs encoding squalene synthase (FIG. 1A), squalene epoxidasel (FIG. 1B), squalene epoxidase 2 (FIG. 1C) and β-amyrin synthase (FIG. 1D).

FIGS. 2A-E. Sequence analysis of M. truncatula genes involved in the early stages of triterpene saponin biosynthesis. (FIG. 2A-FIG. 2C) Dendrograms displaying the sequences of several squalene synthases (SS), squalene epoxidases (SE) and oxidosqualene cyclases (β-amyrin synthase (AS) or cycloartenol synthase (CS)) from plants (Nicotiana tabacum, Nicotiana benthamiana, Panax ginseng, Glycine max, Medicago truncatula, Arabidopsis thaliana, Pisum sativum, Glycyrrhiza echinata), mammals and yeast. The dendrogram was created using the Clustal Sequence Alignment program of the Lasergene software package (DNASTAR, Madison, Wis., USA). (FIG. 2D) Alignments of M. truncatula putative squalene epoxidases 1 and 2 with published squalene epoxidases from other organisms. The highly conserved squalene and FAD binding domains are highlighted by boxes in the N- and C-terminal portions of the proteins, respectively. (FIG. 2E) Alignments of M. truncatula putative β-amyrin synthase with previously reported functionally expressed β-amyrin synthases from pea, licorice and ginseng. A high degree of conservation between the oxidosqualene cyclases can be seen.

FIG. 3. RNA gel blot analysis of tissue distribution of M. truncatula triterpene pathway transcripts. Total RNA was isolated from the tissues shown, resolved by agarose gel electrophoresis, blotted and probed with full length M. truncatula squalene synthase (SS), squalene epoxidase 1 (SE1), squalene epoxidase 2 (SE2) and β-amyrin synthase (β-AS) cDNAs. Cell suspension cultures were of root origin and were induced with yeast elicitor (YE).

FIG. 4. Functional characterization of M. truncatula squalene synthase. M. truncatula squalene synthase (SS) was expressed in E. coli BL21(DE3, pLyS) using the pET-15b expression vector. (A) SDS-PAGE (15 μg protein per lane) showing the induction of the SS protein (˜43 kDa) following exposure of cultures to IPTG. Lanes show separation of proteins from E. coli harboring empty vector (pET-15b) or the SS construct (pET-SS), with analysis of proteins from the culture supernatant (sup) or pellet (ppt). (B) Effect of co-factors on activity of M. truncatula SS expressed in E. coli. The enzyme was assayed by radio-TLC as described in the Examples section below. Lane 1; extract from E. coli harboring pET-15b empty vector assayed in the presence of NADPH+MgCl₂+DTT+KF+¹⁴C-FPP+50 mM Tris-HCl (pH 7.6) (negative control). Lane 2; extract from E. coli harboring pET-SS assayed as in lane 1 (positive control). Lanes 3-11, extracts from E. coli harboring pET-SS assayed with different components in the reaction mixture. Lane 3, without NADPH; lane 4, without DTT; lane 5, without MgCl₂; lanes 6-11, MnCl₂, CaCl₂, CoCl₂, CuCl₂, FeCl₂, ZnCl₂ in place of MgCl₂; lane 12, authentic ¹⁴C-squalene. SQ, squalene; FOH, farnesol.

FIGS. 5A-C. Complementation of the yeast erg1 mutant by M. truncatula squalene epoxidase. (FIG. 5A) Selection of transformants for the Leu⁺ phenotype in SD medium supplied with ergosterol and tryptophan under anaerobic conditions. (FIG. 5B) Plating of yeast cells in YPD (or SD+trp) medium without ergosterol under anaerobic conditions. The transformants were not viable. The same result was obtained with SD medium plus tryptophan. (FIG. 5C) Growth of yeast cells in YPD medium without ergosterol under aerobic conditions. KLN1=non-transformed KLN1 yeast strain; pWV3=KLN1 yeast transformed with the pWV3 yeast expression vector only; pWV3-SE1 and pWV3-SE2=KLN1 yeast transformed with the pWV3 yeast expression vector containing SE1 and SE2 ORFs, respectively; pWV3-SE1Δ47 and pWV3-SE2Δ52=KLN1 yeast transformed with the pWV3 yeast expression vector containing SE1 and SE2, with 47 and 52 amino acids truncated from the N-termini, respectively.

FIGS. 6A-C. Induction of the triterpene pathway in M. truncatula cell suspension cultures exposed to MeJA. (FIG. 6A) Total RNA was isolated from elicited cell cultures at the various times shown, resolved by agarose gel electrophoresis, blotted and hybridized with M. truncatula squalene synthase (SS), squalene epoxidase 2 (SE2), β-amyrin synthase (β-AS), cycloartenol synthase (CAS), phenylalanine ammonia-lyase (PAL) and chalcone synthase (CHS) cDNAs. 18S rRNA was probed as a control for equal loading and transfer of RNA. (FIG. 6B) Blots were quantified by phosphorimager analysis, and data plotted with normalization to the zero time value as 100%. (C, D) Accumulation of triterpene saponins in response to MeJA. The traces show portions of selective ion chromatograms of extracts from unelicited (FIG. 6C) and 24 h MeJA elicited (FIG. 6D) M. truncatula cell suspension cultures. 1, rhamnose-hexose-hexose-hederagenin; 2,3-rhamnose-galactose-glucose-soyasapogenol B; 3, rhamnose-hexose-hexose-soyasapogenol E.

FIG. 7. The biosynthesis of β-amyrin and cycloartenol, and the involvement of cytochrome P450 and glycosyltransferase enzymes in the biosynthesis of the triterpene aglycones and selected conjugates found in M. truncatula.

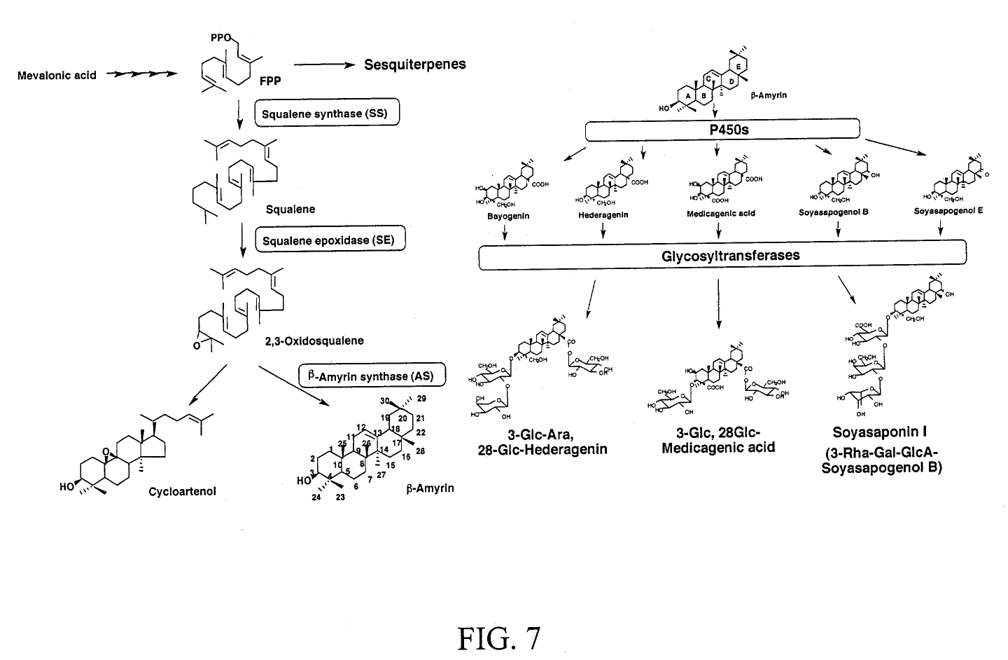

FIGS. 8A-C. Design (FIG. 8A) and example (FIG. 8B—0 hr, FIG. 8C—24 hr) of macroarray used for determination of whether M. truncatula cytochrome P450 and glycosyltransferase genes are induced by methyl jasmonate.

FIGS. 9A-B. Clustering of candidate triterpene pathway P450 (FIG. 9A) and glycosyltransferase (FIG. 9B) genes based on co-expression with β-amyrin synthase in a range of M. truncatula cDNA libraries, estimated by EST counting.

FIG. 10. RNA gel blot analysis to indicate whether candidate triterpene pathway P450 and glycosyltransferase genes are co-induced with β-amyrin synthase (β-AS) in M. truncatula cell cultures exposed to MeJA for the times shown (hours). In each panel, the lower picture shows the ethidium bromide stained gel (check for RNA loading).

FIGS. 11A-B. Phylogenic trees for the top 9 triterpene pathway P450 (FIG. 11A) and GT (FIG. 11B) candidates using ClustalW. The amino acid sequences were deduced using EST analyzer (//bioinfo.noble.org). The consensus sequence from the sequencing data of a given TC (using Lasergene software package DNA Star Madison, Wis., USA) was assembled to the sequence of the corresponding TC and the new consensus sequence was put into the EST analyzer.

### DETAILED DESCRIPTION OF THE INVENTION

The invention overcomes the limitations of the prior art by providing improved methods for the identification of the triterpene biosynthesis genes from legumes. The invention is significant in that many triterpenes produced by legumes are known to have medicinal uses. Isolation of genes in the biosynthetic pathway of triterpenes produced by legumes will thus allow the use of biotechnological approaches to modifying triterpene biosynthesis in legumes and other plants. By introduction of one or more of these genes, production of legume triterpenes may be obtained in plants otherwise lacking the triterpenes, thereby providing the associated health benefits. Isolation triterpene biosynthesis genes also provides the potential for decreasing the production of one or more triterpenes in plants, for example, by use of antisense technology. As some triterpenes can be toxic to monogastric animals, act as anti-palatability factors, or negatively impact forage digestibility in ruminants, the ability to selectively decrease triterpene production is significant.

The invention relates to the finding that, in legumes, triterpene biosynthesis is upregulated in the presence of methyl jasmonate. This is important because triterpenes are normally produced at low basal levels in cultured cells of legumes. In order to implement high-throughput techniques to identify triterpene biosynthesis genes, it is necessary to develop a system in which the saponin pathway can be rapidly and reproducibly induced from basal levels. Extraction and quantitation of triterpenes can be difficult and therefore does not represent the best assay method for determining expression of the triterpene pathway. The approach of the inventors overcomes these limitations by allowing analysis of changes in transcript levels following treatment with methyl jasmonate. Thus the invention allows, for example, identifying a triterpene biosynthesis gene by contacting a plant cell of a legume with methyl jasmonate and identifying a coding sequence which is specifically upregulated in the cell following the contacting with methyl jasmonate. The technique is amenable to the use of high-throughput technology, such as the use of arrays, or so-called “gene chips.” In this manner, one or more triterpene biosynthesis genes can be rapidly identified.

The invention further provides triterpene biosynthesis genes. Specifically provided herein are the squalene epoxidase, squalene synthase and β-amyrin synthase coding sequences (for example, SEQ ID NO:2, SEQ ID NO:4 and SEQ ID NO:6), which were initially isolated from Medicago truncatula. One embodiment of the invention thus provides these nucleic acids, nucleic acids encoding the same polypeptides as these sequences, and sequences hybridizing to these nucleic acids and having squalene epoxidase, squalene synthase or β-amyrin synthase activity, respectively. These nucleic acids may find use in the creation of genetically engineered plants with altered triterpene biosynthesis, as is described herein below. Further provided by the invention is the promoter region of the Medicago sativa squalene epoxidase gene (SEQ ID NO:1). Therefore, the invention provides, in one embodiment, a squalene epoxidase promoter comprising the nucleic acid sequence of the promoter region in SEQ ID NO:1, or a fragment thereof having promoter activity. This promoter may find particular utility in the expression of transgenes based on the expression profile of the squalene epoxidase gene.

The methods of the invention are amenable to an EST data mining approach for isolation of candidate triterpene biosynthesis genes and the functional identification of these genes by heterologous expression in E. coli or yeast. For example, corresponding cDNA sequences may be identified by the approach and used as probes for development of an inducible cell culture system for triterpene pathway gene discovery by bioinformatic and DNA array-based approaches, and a number of candidate saponin pathway cytochrome P450 and glycosyltransferase genes identified.

I. Gene Expression Assays

One aspect of the invention comprises use of assays for detecting the expression of one or more triterpene biosynthesis genes and to facilitate the characterization of these genes. Such assays may be carried out using whole plants, plant parts or cultured cells. An advantage of using cellular assays with the current invention is that cellular growth conditions can be more readily controlled and treatment with methyl jasmonate can be carried out more effectively.

The biological sample to be assayed may comprise nucleic acids isolated from the cells of any plant material according to standard methodologies (Sambrook et al., 2001). In one embodiment of the invention, the nucleic acid may be fractionated or whole cell RNA. Where RNA is used, it may be desired to convert the RNA to a complementary DNA. In one embodiment of the invention, the RNA is whole cell RNA; in another, it is poly-A RNA. Commonly, the nucleic acid may be amplified for assaying.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Following detection, one may compare the results seen in a given plant with a statistically significant reference group of non-transformed control plants. For example, the results obtained with and without treatment with methyl jasmonate may be compared to identify one or more triterpene biosynthesis genes upregulated in the response to the treatment. Preferably, the control plants or cells are of a genetic background similar to the test plant and/or cells. In this way, it is possible to detect differences in the amount or kind of protein detected in test plants and the responsible coding sequences can be identified. Alternatively, clonal cultures of cells, for example, suspension cultures or an immature embryo, may be compared to other cell samples.

As indicated, a variety of different assays are contemplated in the screening of cells or plants according to the invention. These techniques may be used to detect the expression of particular triterpene biosynthesis genes and identify the corresponding coding sequences. The techniques include but are not limited to, direct DNA sequencing, pulsed field gel electrophoresis (PFGE) analysis, Southern or Northern blotting, single-stranded conformation analysis (SSCA), RNAse protection assay, allele-specific oligonucleotide (ASO), dot blot analysis, denaturing gradient gel electrophoresis, RFLP and PCR™-SSCP.

A. Arrays

Arrays may be used for the detection of differential expression of a triterpene biosynthesis gene in accordance with the invention. For example, by hybridizing differentially labeled RNA or DNA taken from cells treated or not treated with methyl jasmonate to an array, loci corresponding to the differentially expressed sequences can be identified. Using, for instance, two different fluorescent labels, the relative proportion of nucleic acid sequences in the test and control samples can be determined for any given nucleic acid based on the color of the signal yielded by hybridization to that nucleic acid.

Arrays may comprise nucleic acids corresponding to a plurality of coding sequences arranged on a solid support. The use of arrays involves the placement and binding of nucleic acids to known locations, termed sectors, on a solid support. Arrays can be used, through hybridization of test and control samples to the array, to determine the presence or absence of a given molecule in the sample and/or the relative concentrations of the molecule. By including multiple target nucleic acids on an array, potentially thousands of target molecules can be simultaneously screened for in a test sample. Many different methods for preparation of arrays comprising target nucleic acids arranged on solid supports are known to those of skill in the art and could be used in accordance with the invention. Specific methods for preparation of such arrays are disclosed in, for example, Immobilized Biochemicals and Affinity Chromatography, 1974; U.S. Pat. Nos. 6,287,768; 6,077,673; and 5,994,076, each specifically incorporated herein by reference in its entirety. Examples of other techniques which have been described for the attachment of test materials to arrays include the use of successive application of multiple layers of biotin, avidin, and extenders (U.S. Pat. No. 4,282,287, specifically incorporated herein by reference in its entirety); methods employing a photochemically active reagent and a coupling agent which attaches the photoreagent to the substrate (U.S. Pat. No. 4,542,102, specifically incorporated herein by reference in its entirety); use of polyacrylamide supports on which are immobilized oligonucleotides (PCT Patent Publication No. 90/07582, specifically incorporated herein by reference in its entirety); use of solid supports on which oligonucleotides are immobilized via a 5′-dithio linkage (PCT Patent Publication No. 91/00868, specifically incorporated herein by reference in its entirety); and through use of a photoactivateable derivative of biotin as the agent for immobilizing a biological polymer of interest onto a solid support (see U.S. Pat. No. 5,252,743; and PCT Patent Publication No. 91/07087 to Barrett et al., each specifically incorporated herein by reference in its entirety). In the case of a solid support made of nitrocellulose or the like, standard techniques for UV-crosslinking may be of particular utility (Sambrook et al., 2001).

The solid support surface upon which an array is produced in accordance with the invention may potentially be any suitable substance. Examples of materials which may be used include polymers, plastics, resins, polysaccharides, silica or silica-based materials, carbon, metals, inorganic glasses, membranes, etc. It may also be advantageous to use a surface which is optically transparent, such as flat glass or a thin layer of single-crystal silicon. Surfaces on the solid substrate will usually, though not always, be composed of the same material as the substrate, and the surface may further contain reactive groups, which could be carboxyl, amino, hydroxyl, or the like.

It is contemplated that one may wish to use a solid support surface which is provided with a layer of crosslinking groups (U.S. Pat. No. 5,412,087, specifically incorporated herein by reference in its entirety). Crosslinking groups could be selected from any suitable class of compounds, for example, aryl acetylenes, ethylene glycol oligomers containing 2 to 10 monomer units, diamines, diacids, amino acids, or combinations thereof. Crosslinking groups can be attached to the surface by a variety of methods that will be readily apparent to one of skill in the art. For example, crosslinking groups may be attached to the surface by siloxane bonds formed via reactions of crosslinking groups bearing trichlorosilyl or trisalkoxy groups with hydroxyl groups on the surface of the substrate. The crosslinking groups can be attached in an ordered array, i.e., as parts of the head groups in a polymerized Langmuir Blodgett film. The linking groups may be attached by a variety of methods that are readily apparent to one skilled in the art, for instance, by esterification or amidation reactions of an activated ester of the linking group with a reactive hydroxyl or amine on the free end of the crosslinking group.

A significant benefit of the arrays of the invention is that they may be used to simultaneously screen individuals or biological samples therefrom for expression of a plurality of triterpene biosynthesis genes. Use of the arrays generally will comprise, in a first step, contacting the array with a test sample and/or a control sample. Generally the test sample will be labeled to facilitate detection of hybridizing test samples. By detection of test samples having affinity for bound target nucleic acids or other ligands, the identity of the target molecule will be known.

Following contacting with the test sample, the solid support surface is then generally washed free of unbound test sample, and the signal corresponding to the probe label is identified for those regions on the surface where the test sample has high affinity. Suitable labels for the test sample include, but are not limited to, radiolabels, chromophores, fluorophores, chemiluminescent moieties, antigens and transition metals. In the case of a fluorescent label, detection can be accomplished with a charge-coupled device (CCD), fluorescence microscopy, or laser scanning (U.S. Pat. No. 5,445,934, specifically incorporated herein by reference in its entirety). When autoradiography is the detection method used, the marker is a radioactive label, such as ³²P, and the surface is exposed to X-ray film, which is developed and read out on a scanner or, alternatively, simply scored manually. With radiolabeled probes, exposure time will typically range from one hour to several days. Fluorescence detection using a fluorophore label, such as fluorescein, attached to the ligand will usually require shorter exposure times. Alternatively, the presence of a bound probe may be detected using a variety of other techniques, such as an assay with a labeled enzyme, antibody, or the like. Detection also may, in the case of nucleic acids, alternatively be carried out using PCR. In this instance, PCR detection may be carried out in situ on the slide. In this case one may wish to utilize one or more labeled nucleotides in the PCR mix to produce a detectable signal. Other techniques using various marker systems for detecting bound ligand will also be readily apparent to those skilled in the art.

B. Nucleic Acid Amplification Reaction

Nucleic acid molecules can be detected using a variety of techniques, including amplification reactions. The present invention contemplates using these amplification reactions for detecting expression of a triterpene biosynthesis gene. Nucleic acid used as a template for amplification can be isolated from cells contained in the biological sample, according to standard methodologies (Sambrook, 2001). The nucleic acid may be genomic DNA or fractionated or whole cell RNA. Where RNA is used, it may be desired to convert the RNA to a cDNA.

Pairs of primers that selectively hybridize to nucleic acids are contacted with the isolated nucleic acid under conditions that permit selective hybridization. The term “primer,” as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred.

Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced.

Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals (Affymax technology).

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and each incorporated herein by reference in entirety.

C. Quantitation of Gene Expression with Relative Quantitative RT-PCR™

Reverse transcription (RT) of RNA to cDNA followed by relative quantitative PCR™ (RT-PCR™) can be used to determine the relative concentrations of specific mRNA species expressed by cells. By determining that the concentration of a specific mRNA species varies, it is shown that the gene encoding the specific mRNA species is differentially expressed. In accordance with the invention, differential expression between cells treated or not treated with methyl jasmonate can be used to identify triterpene biosynthesis genes.

In PCR™, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR™ amplification is directly proportional to the starting concentration of the target before the reaction began. By determining the concentration of the amplified products of the target DNA in PCR™ reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundance of the specific mRNA from which the target sequence was derived can be determined for the respective tissues or cells. This direct proportionality between the concentration of the PCR™ products and the relative mRNA abundance is only true in the linear range of the PCR™ reaction.

The final concentration of the target DNA in the plateau portion of the curve is determined by the availability of reagents in the reaction mix and is independent of the original concentration of target DNA. Therefore, the first condition that must be met before the relative abundance of a mRNA species can be determined by RT-PCR™ for a collection of RNA populations is that the concentrations of the amplified PCR™ products must be sampled when the PCR™ reactions are in the linear portion of their curves.

The second condition that must be met for an RT-PCR™ study to successfully determine the relative abundance of a particular mRNA species is that relative concentrations of the amplifiable cDNAs must be normalized to some independent standard. The goal of an RT-PCR™ study is to determine the abundance of a particular mRNA species relative to the average abundance of all mRNA species in the sample.

Most protocols for competitive PCR™ utilize internal PCR™ standards that are approximately as abundant as the target. These strategies are effective if the products of the PCR™ amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR™ assay for plant tissue. The problems inherent in plant tissue samples are that they can be of variable quantity (making normalization problematic) and quality (potentially necessitating the co-amplification of a reliable internal control, preferably of larger size than the target). Both of these problems are overcome if the RT-PCR™ is performed as a relative quantitative RT-PCR™ with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies may be performed using a more conventional relative quantitative RT-PCR™ assay with an external standard protocol. These assays sample the PCR™ products in the linear portion of their amplification curves. The number of PCR™ cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR™ assays can be superior to those derived from the relative quantitative RT-PCR™ assay with an internal standard.

One reason for this advantage is that without the internal standard/competitor, all of the reagents can be converted into a single PCR™ product in the linear range of the amplification curve, thus increasing the sensitivity of the assay. Another reason is that with only one PCR™ product, display of the product on an electrophoretic gel or another display method becomes less complex, has less background and is easier to interpret.

D. Purification and Assays of Proteins

Another means for confirming the expression of a given coding sequence is to purify and quantify a polypeptide expressed by the coding sequence and/or the end product that is biosynthesized by the coding sequence. For example, the identity of a triterpene biosynthesis gene can be confirmed by the production of a product catalyzed by the gene product either in vivo or in vitro. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; and isoelectric focusing. A particularly efficient method of purifying peptides is fast protein liquid chromatography or even HPLC.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide being assayed always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

E. Immunological Detection

1. Immunoassays

Immunoassays may find use with the invention in certain prognostic/diagnostic applications that comprise assaying for the presence of triterpene biosynthesis polypeptides. The steps of various useful immunodetection methods have been described in the scientific literature, such as, e.g., Nakamura et al. (1987; incorporated herein by reference). Immunoassays, in their most simple and direct sense, are binding assays. Certain preferred immunoassays are the various types of enzyme linked immunosorbent assays (ELISAs), radioimmunoassays (RIA) and immunobead capture assay. Immunohistochemical detection using tissue sections also is particularly useful. However, it will be readily appreciated that detection is not limited to such techniques, and Western blotting, dot blotting, FACS analyses, and the like also may be used in connection with the present invention.

In general, immunobinding methods include obtaining a sample suspected of containing a protein, peptide or antibody, and contacting the sample with an antibody or protein or peptide in accordance with the present invention, as the case may be, under conditions effective to allow the formation of immunocomplexes.

The immunobinding methods of this invention include methods for detecting or quantifying the amount of a reactive component in a sample, which methods require the detection or quantitation of any immune complexes formed during the binding process. Here, one would obtain a sample containing a target protein or peptide, and contact the sample with an antibody, as the case may be, and then detect or quantify the amount of immune complexes formed under the specific conditions.

Contacting the chosen biological sample with the protein, peptide or antibody under conditions effective and for a period of time sufficient to allow the formation of immune complexes (primary immune complexes) is generally a matter of simply adding the composition to the sample and incubating the mixture for a period of time long enough for the antibodies to form immune complexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference.

The immunodetection methods of the present invention have evident utility in the diagnosis of cancer. Here, a biological or clinical sample suspected of containing either the encoded protein or peptide or corresponding antibody is used. However, these embodiments also have applications to non-clinical samples, such as in the titering of antigen or antibody samples, in the selection of hybridomas, and the like.

2. ELISAs

In one exemplary ELISA, antibodies binding to the encoded proteins of the invention are immobilized onto a selected surface exhibiting protein affinity, such as a well in a polystyrene microtiter plate. After binding and washing to remove non-specifically bound immunocomplexes, the bound antigen may be detected. Detection is generally achieved by the addition of a second antibody specific for the target protein, that is linked to a detectable label. This type of ELISA is a simple “sandwich ELISA”. Detection also may be achieved by the addition of a second antibody, followed by the addition of a third antibody that has binding affinity for the second antibody, with the third antibody being linked to a detectable label.

In another exemplary ELISA, the samples are immobilized onto the well surface and then contacted with the appropriate antibodies. After binding and washing to remove non-specifically bound immunecomplexes, the bound antibody is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Irrespective of the format employed, ELISAs have certain features in common, such as coating, incubating or binding, washing to remove non-specifically bound species, and detecting the bound immunecomplexes. These are described as follows:

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control human cancer and/or clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

“Under conditions effective to allow immunecomplex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 h, at temperatures preferably on the order of 25° to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immunecomplex with a urease, glucose oxidase, alkaline phosphatase or hydrogen peroxidase-conjugated antibody for a period of time and under conditions that favor the development of further immunecomplex formation (e.g., incubation for 2 h at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation is then achieved by measuring the degree of color generation, e.g., using a visible spectra spectrophotometer.

In other embodiments, solution-phase competition ELISA is also contemplated. Solution phase ELISA involves attachment of the target protein to a bead, for example a magnetic bead. The bead is then incubated with sera from human and animal origin. After a suitable incubation period to allow for specific interactions to occur, the beads are washed. The specific type of antibody is then detected with an antibody indicator conjugate. The beads are washed and sorted. This complex is then read on an appropriate instrument (fluorescent, electroluminescent, spectrophotometer, depending on the conjugating moiety). The level of antibody binding can thus by quantitated and is directly related to the amount of signal present.

II. Plant Transformation Constructs

Certain embodiments of the current invention concern plant transformation constructs. For example, one aspect of the current invention is a plant transformation vector comprising one or more triterpene biosynthesis gene, including squalene epoxidase, squalene synthase and β-amyrin synthase coding sequences. Also provided are plant transformation vectors comprising a coding sequence operatively linked to a promoter sequence from a triterpene biosynthesis gene. One promoter provided by the invention is the Medicago sativa squalene epoxidase promoter (SEQ ID NO:1). Such sequences may be isolated by the methods of the invention.

Exemplary coding sequences for use with the invention include the squalene epoxidase, squalene synthase and β-amyrin synthase coding sequences from Medicago truncatula, the nucleic acid sequences of which are provided by SEQ ID NO:2, SEQ ID NO:4 and SEQ ID NO:6, respectively. Also provided by the invention are nucleic acid sequences encoding the polypeptide sequences encoded by SEQ ID NO:3, SEQ ID NO:5 and SEQ ID NO:7. Further provided are the coding sequences given in each of SEQ ID NOs:18-31. In certain embodiments of the invention, these sequences are provided operably linked to a heterologous promoter, in either sense or antisense orientation. Expression constructs are also provided comprising these sequences, as are plants and plant cells transformed with the sequences. Further provided are methods of modifying triterpene biosynthesis comprising introducing one or more of these coding sequences into a plant cell, including a whole plant.

The construction of vectors which may be employed in conjunction with plant transformation techniques using these or other sequences according to the invention will be known to those of skill of the art in light of the present disclosure (see, for example, Sambrook et al., 2001; Gelvin et al., 1990). The techniques of the current invention are thus not limited to any particular nucleic acid sequences.

One important use of the sequences provided by the invention will be in the alteration of plant phenotypes by genetic transformation of plants with sense or antisense triterpene biosynthesis genes. The triterpene biosynthesis gene may be provided with other sequences. Where an expressible coding region that is not necessarily a marker coding region is employed in combination with a marker coding region, one may employ the separate coding regions on either the same or different DNA segments for transformation. In the latter case, the different vectors are delivered concurrently to recipient cells to maximize cotransformation.

The choice of any additional elements used in conjunction with the triterpene biosynthesis coding or promoter sequences will often depend on the purpose of the transformation. One of the major purposes of transformation of crop plants is to add commercially desirable, agronomically important traits to the plant. As triterpenes are known to confer many beneficial effects on health, one such trait is increased biosynthesis of triterpenes. Alternatively, plants may be engineered to decrease synthesis of triterpenes. This may be beneficial, for example, to improve the taste of a food to humans or animals. For instance, poultry will not eat feed containing certain triterpenes.

Vectors used for plant transformation may include, for example, plasmids, cosmids, YACs (yeast artificial chromosomes), BACs (bacterial artificial chromosomes) or any other suitable cloning system, as well as fragments of DNA therefrom. Thus when the term “vector” or “expression vector” is used, all of the foregoing types of vectors, as well as nucleic acid sequences isolated therefrom, are included. It is contemplated that utilization of cloning systems with large insert capacities will allow introduction of large DNA sequences comprising more than one selected gene. In accordance with the invention, this could be used to introduce genes corresponding to the entire triterpene biosynthetic pathway into a plant. Introduction of such sequences may be facilitated by use of bacterial or yeast artificial chromosomes (BACs or YACs, respectively), or even plant artificial chromosomes. For example, the use of BACs for Agrobacterium-mediated transformation was disclosed by Hamilton et al. (1996).

Particularly useful for transformation are expression cassettes which have been isolated from such vectors. DNA segments used for transforming plant cells will, of course, generally comprise the cDNA, gene or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. However, this may not always be the case, and the present invention also encompasses transgenic plants incorporating non-expressed transgenes. Preferred components likely to be included with vectors used in the current invention are as follows.

A. Regulatory Elements

Exemplary promoters for expression of a nucleic acid sequence include plant promoter such as the CaMV 35S promoter (Odell et al., 1985), or others such as CaMV 19S (Lawton et al., 1987), nos (Ebert et al., 1987), Adh (Walker et al., 1987), sucrose synthase (Yang and Russell, 1990), a-tubulin, actin (Wang et al., 1992), cab (Sullivan et al., 1989), PEPCase (Hudspeth and Grula, 1989) or those associated with the R gene complex (Chandler et al., 1989). Tissue specific promoters such as root cell promoters (Conkling et al., 1990) and tissue specific enhancers (Fromm et al., 1986) are also contemplated to be particularly useful, as are inducible promoters such as ABA- and turgor-inducible promoters.

One preferred promoter is the Medicago sativa squalene epoxidase promoter (SEQ ID NO:1). Thus one aspect of the invention provides the nucleic acid sequence of SEQ ID NO:1 or fragments thereof having promoter activity, as well as vectors comprising this sequence. Preferably, the promoter is linked to a coding sequence.

The DNA sequence between the transcription initiation site and the start of the coding sequence, i.e., the untranslated leader sequence, can also influence gene expression. One may thus wish to employ a particular leader sequence with a transformation construct of the invention. Preferred leader sequences are contemplated to include those which include sequences predicted to direct optimum expression of the attached gene, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants, and in tomato in particular, will be most preferred.

It is contemplated that vectors for use in accordance with the present invention may be constructed to include the ocs enhancer element. This element was first identified as a 16 bp palindromic enhancer from the octopine synthase (ocs) gene of Agrobacterium (Ellis et al., 1987), and is present in at least 10 other promoters (Bouchez et al., 1989). It is proposed that the use of an enhancer element, such as the ocs element and particularly multiple copies of the element, will act to increase the level of transcription from adjacent promoters when applied in the context of plant transformation.

It is specifically envisioned that triterpene biosynthesis coding sequences may be introduced under the control of novel promoters or enhancers, etc., or perhaps even homologous or tissue specific promoters or control elements. Vectors for use in tissue-specific targeting of genes in transgenic plants will typically include tissue-specific promoters and may also include other tissue-specific control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters which have higher activity in roots or wounded leaf tissue; a truncated (−90 to +8) 35S promoter which directs enhanced expression in roots, and an a-tubulin gene that directs expression in roots.

B. Terminators

Transformation constructs prepared in accordance with the invention will typically include a 3′ end DNA sequence that acts as a signal to terminate transcription and allow for the poly-adenylation of the mRNA produced by coding sequences operably linked to a triterpene biosynthesis gene. In one embodiment of the invention, the native promoter of the triterpene biosynthesis gene is used. Alternatively, a heterologous 3′ end may enhance the expression of sense or antisense triterpene biosynthesis genes. Terminators which are deemed to be particularly useful in this context include those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Regulatory elements such as Adh intron (Callis et al., 1987), sucrose synthase intron (Vasil et al., 1989) or TMV omega element (Gallie et al., 1989), may further be included where desired.

C. Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

D. Marker Genes

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening”′ (e.g., the green fluorescent protein). Of course, many examples of suitable marker proteins are known to the art and can be employed in the practice of the invention.

Included within the terms selectable or screenable markers also are genes which encode a “secretable marker” whose secretion can be detected as a means of identifying or selecting for transformed cells. Examples include markers which are secretable antigens that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR-S).

With regard to selectable secretable markers, the use of a gene that encodes a protein that becomes sequestered in the cell wall, and which protein includes a unique epitope is considered to be particularly advantageous. Such a secreted antigen marker would ideally employ an epitope sequence that would provide low background in plant tissue, a promoter-leader sequence that would impart efficient expression and targeting across the plasma membrane, and would produce protein that is bound in the cell wall and yet accessible to antibodies. A normally secreted wall protein modified to include a unique epitope would satisfy all such requirements.

Many selectable marker coding regions are known and could be used with the present invention including, but not limited to, neo (Potrykus et al., 1985), which provides kanamycin resistance and can be selected for using kanamycin, G418, paromomycin, etc.; bar, which confers bialaphos or phosphinothricin resistance; a mutant EPSP synthase protein (Hinchee et al., 1988) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (European Patent Application 154,204, 1985); a methotrexate resistant DHFR (Thillet et al., 1988), a dalapon dehalogenase that confers resistance to the herbicide dalapon; or a mutated anthranilate synthase that confers resistance to 5-methyl tryptophan. Where a mutant EPSP synthase is employed, additional benefit may be realized through the incorporation of a suitable chloroplast transit peptide, CTP (U.S. Pat. No. 5,188,642) or OTP (U.S. Pat. No. 5,633,448) and use of a modified maize EPSPS (PCT Application WO 97/04103).

An illustrative embodiment of selectable marker capable of being used in systems to select transformants are those that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death.

Where one desires to employ a bialaphos resistance gene in the practice of the invention, the inventor has discovered that particularly useful genes for this purpose are the bar or pat genes obtainable from species of Streptomyces (e.g., ATCC No. 21,705). The cloning of the bar gene has been described (Murakami et al., 1986; Thompson et al., 1987) as has the use of the bar gene in the context of plants (De Block et al., 1987; De Block et al., 1989; U.S. Pat. No. 5,550,318).

Screenable markers that may be employed include a β-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a β-lactamase gene (Sutcliffe, 1978), which encodes an enzyme for which various chromogenic substrates are known (e.g., PADAC, a chromogenic cephalosporin); a xylE gene (Zukowsky et al., 1983) which encodes a catechol dioxygenase that can convert chromogenic catechols; an α-amylase gene (Ikuta et al., 1990); a tyrosinase gene (Katz et al., 1983) which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene (Ow et al., 1986), which allows for bioluminescence detection; an aequorin gene (Prasher et al., 1985) which may be employed in calcium-sensitive bioluminescence detection; or a gene encoding for green fluorescent protein (Sheen et al., 1995; Haseloff et al., 1997; Reichel et al., 1996; Tian et al., 1997; WO 97/41228).

Another screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It also is envisioned that this system may be developed for populational screening for bioluminescence, such as on tissue culture plates, or even for whole plant screening. The gene which encodes green fluorescent protein (GFP) is contemplated as a particularly useful reporter gene (Sheen et al., 1995; Haseloff et al., 1997; Reichel et al., 1996; Tian et al., 1997; WO 97/41228). Expression of green fluorescent protein may be visualized in a cell or plant as fluorescence following illumination by particular wavelengths of light. Where use of a screenable marker gene such as lux or GFP is desired, the inventors contemplated that benefit may be realized by creating a gene fusion between the screenable marker gene and a selectable marker gene, for example, a GFP-NPTII gene fusion. This could allow, for example, selection of transformed cells followed by screening of transgenic plants or seeds.

III. Antisense Constructs

Antisense treatments are one way of altering triterpene biosynthesis in accordance with the invention. In particular, constructs comprising a triterpene biosynthesis gene and/or a promoter thereof, including the Medicago truncatula squalene epoxidase, squalene synthase and β-amyrin synthase coding sequences provided herein, in antisense orientation may be used to decrease or effectively eliminate the expression of one or more triterpenes in a plant. As such, antisense technology may be used to “knock-out” the function of a triterpene biosynthesis gene or homologous sequences thereof.

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see above) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

IV. Tissue Cultures

Tissue cultures represent one convenient means of obtaining cells for use in the assays of the invention. Growth of the cells in tissue cultures allows maintenance of a continuous source of plant cells produced under uniform conditions and allows careful control of methyl jasmonate administration. Maintenance of tissue cultures requires use of media and controlled environments. “Media” refers to the numerous nutrient mixtures that are used to grow cells in vitro, that is, outside of the intact living organism. The medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

Nutrient media is prepared as a liquid, but this may be solidified by adding the liquid to materials capable of providing a solid support. Agar is most commonly used for this purpose. Bactoagar, Hazelton agar, Gelrite, and Gelgro are specific types of solid support that are suitable for growth of plant cells in tissue culture.

Some cell types will grow and divide either in liquid suspension or on solid media. As disclosed herein, plant cells will grow in suspension or on solid medium, but regeneration of plants from suspension cultures typically requires transfer from liquid to solid media at some point in development. The type and extent of differentiation of cells in culture will be affected not only by the type of media used and by the environment, for example, pH, but also by whether media is solid or liquid.

Tissue that can be grown in a culture includes meristem cells, Type I, Type II, and Type III callus, immature embryos and gametic cells such as microspores, pollen, sperm and egg cells. Type I, Type II, and Type III callus may be initiated from tissue sources including, but not limited to, immature embryos, seedling apical meristems, root, leaf, microspores and the like. Those cells which are capable of proliferating as callus also are recipient cells for genetic transformation.

Somatic cells are of various types. Embryogenic cells are one example of somatic cells which may be induced to regenerate a plant through embryo formation. Non-embryogenic cells are those which typically will not respond in such a fashion. Certain techniques may be used that enrich recipient cells within a cell population. For example, Type II callus development, followed by manual selection and culture of friable, embryogenic tissue, generally results in an enrichment of cells. Manual selection techniques which can be employed to select target cells may include, e.g., assessing cell morphology and differentiation, or may use various physical or biological means. Cryopreservation also is a possible method of selecting for recipient cells.

Manual selection of recipient cells, e.g., by selecting embryogenic cells from the surface of a Type II callus, is one means that may be used in an attempt to enrich for particular cells prior to culturing (whether cultured on solid media or in suspension).

Where employed, cultured cells may be grown either on solid supports or in the form of liquid suspensions. In either instance, nutrients may be provided to the cells in the form of media, and environmental conditions controlled. There are many types of tissue culture media comprised of various amino acids, salts, sugars, growth regulators and vitamins. Most of the media employed in the practice of the invention will have some similar components, but may differ in the composition and proportions of their ingredients depending on the particular application envisioned. For example, various cell types usually grow in more than one type of media, but will exhibit different growth rates and different morphologies, depending on the growth media. In some media, cells survive but do not divide. Various types of media suitable for culture of plant cells previously have been described. Examples of these media include, but are not limited to, the N6 medium described by Chu et al. (1975) and MS media (Murashige and Skoog, 1962).

V. Methods for Genetic Transformation

Suitable methods for transformation of plant or other cells for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety), by Agrobacterium-mediated transformation (U.S. Pat. No. 5,591,616 and U.S. Pat. No. 5,563,055; both specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference in its entirety), etc. Through the application of techniques such as these, the cells of virtually any plant species may be stably transformed, and these cells developed into transgenic plants.

A. Agrobacterium-mediated Transformation

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety.

Agrobacterium-mediated transformation is most efficient in dicotyledonous plants and is the preferable method for transformation of dicots, including Arabidopsis, tobacco, tomato, alfalfa, and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., 1997; U.S. Pat. No. 5,591,616, specifically incorporated herein by reference in its entirety), wheat (McCormac et al., 1998), barley (Tingay et al., 1997; McCormac et al., 1998), alfalfa (Thomas et al., 1990) and maize (Ishidia et al., 1996).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described (Rogers et al., 1987) have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

B. Electroporation

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plants (Bates, 1994; Lazzeri, 1995). For example, the generation of transgenic soybean plants by electroporation of cotyledon-derived protoplasts is described by Dhir and Widholm in Intl. Patent Appl. Publ. No. WO 9217598 (specifically incorporated herein by reference). Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

C. Microprojectile Bombardment

Another method for delivering transforming DNA segments to plant cells in accordance with the invention is microprojectile bombardment (U.S. Pat. Nos. 5,550,318; 5,538,880; 5,610,042; and PCT Application WO 94/09699; each of which is specifically incorporated herein by reference in its entirety). In this method, particles may be coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

Microprojectile bombardment techniques are widely applicable, and may be used to transform virtually any plant species. Examples of species for which have been transformed by microprojectile bombardment include monocot species such as maize (PCT Application WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casa et al., 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al. 1994), peanut (Singsit et al., 1997), cotton (McCabe and Martinell, 1993), tomato (VanEck et al. 1995), and legumes in general (U.S. Pat. No. 5,563,055, specifically incorporated herein by reference in its entirety).

D. Other Transformation Methods

Transformation of protoplasts can be achieved using methods based on calcium phosphate precipitation, polyethylene glycol treatment, electroporation, and combinations of these treatments (see, e.g., Potrykus et al., 1985; Lorz et al., 1985; Omirulleh et al., 1993; Fromm et al., 1986; Uchimiya et al, 1986; Callis et al., 1987; Marcotte et al., 1988).

Application of these systems to different plant strains depends upon the ability to regenerate that particular plant strain from protoplasts. Illustrative methods for the regeneration of cereals from protoplasts have been described (Toriyama et al., 1986; Yamada et al., 1986; Abdullah et al., 1986; Omirulleh et al., 1993 and U.S. Pat. No. 5,508,184; each specifically incorporated herein by reference in its entirety). Examples of the use of direct uptake transformation of cereal protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

To transform plant strains that cannot be successfully regenerated from protoplasts, other ways to introduce DNA into intact cells or tissues can be utilized. For example, regeneration of cereals from immature embryos or explants can be effected as described (Vasil, 1989). Also, silicon carbide fiber-mediated transformation may be used with or without protoplasting (Kaeppler, 1990; Kaeppler et al., 1992; U.S. Pat. No. 5,563,055, specifically incorporated herein by reference in its entirety). Transformation with this technique is accomplished by agitating silicon carbide fibers together with cells in a DNA solution. DNA passively enters as the cells are punctured. This technique has been used successfully with, for example, the monocot cereals maize (PCT Application WO 95/06128, specifically incorporated herein by reference in its entirety; (Thompson, 1995) and rice (Nagatani, 1997).

VIII. Production and Characterization of Stably Transformed Plants

After effecting delivery of exogenous DNA to recipient cells, the next steps generally concern identifying the transformed cells for further culturing and plant regeneration. As mentioned herein, in order to improve the ability to identify transformants, one may desire to employ a selectable or screenable marker gene with a transformation vector prepared in accordance with the invention. In this case, one would then generally assay the potentially transformed cell population by exposing the cells to a selective agent or agents, or one would screen the cells for the desired marker gene trait.

A. Selection

It is believed that DNA is introduced into only a small percentage of target cells in any one experiment. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphostransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

One herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., 1973). Synthetic PPT, the active ingredient in the herbicide Liberty™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., 1987). The bar gene has been cloned (Murakami et al., 1986; Thompson et al., 1987) and expressed in transgenic tobacco, tomato, potato (De Block et al., 1987) Brassica (De Block et al., 1989) and maize (U.S. Pat. No. 5,550,318). In previous reports, some transgenic plants which expressed the resistance gene were completely resistant to commercial formulations of PPT and bialaphos in greenhouses.

Another example of a herbicide which is useful for selection of transformed cell lines in the practice of the invention is the broad spectrum herbicide glyphosate. Glyphosate inhibits the action of the enzyme EPSPS which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103. The best characterized mutant EPSPS gene conferring glyphosate resistance comprises amino acid changes at residues 102 and 106, although it is anticipated that other mutations will also be useful (PCT/WO97/4103).

To use the bar-bialaphos or the EPSPS-glyphosate selective system, bombarded tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is proposed that ranges of 0.1-50 mg/l bialaphos or 0.1-50 mM glyphosate will find utility in the practice of the invention. Tissue can be placed on any porous, inert, solid or semi-solid support for bombardment, including but not limited to filters and solid culture medium. Bialaphos and glyphosate are provided as examples of agents suitable for selection of transformants, but the technique of this invention is not limited to them.

It further is contemplated that the herbicide DALAPON, 2,2-dichloropropionic acid, may be useful for identification of transformed cells. The enzyme 2,2-dichloropropionic acid dehalogenase (deh) inactivates the herbicidal activity of 2,2-dichloropropionic acid and therefore confers herbicidal resistance on cells or plants expressing a gene encoding the dehalogenase enzyme (Buchanan-Wollaston et al., 1992; U.S. Pat. No. 5,508,468; each of the disclosures of which is specifically incorporated herein by reference in its entirety).

Alternatively, a gene encoding anthranilate synthase, which confers resistance to certain amino acid analogs, e.g., 5-methyltryptophan or 6-methyl anthranilate, may be useful as a selectable marker gene. The use of an anthranilate synthase gene as a selectable marker was described in U.S. Pat. No. 5,508,468.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. The R-locus is useful for selection of transformants from bombarded immature embryos. In a similar fashion, the introduction of the C1 and B genes will result in pigmented cells and/or tissues.

The enzyme luciferase may be used as a screenable marker in the context of the present invention. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time. Another screenable marker which may be used in a similar fashion is the gene coding for green fluorescent protein.

It further is contemplated that combinations of screenable and selectable markers will be useful for identification of transformed cells. In some cell or tissue types a selection agent, such as bialaphos or glyphosate, may either not provide enough killing activity to clearly recognize transformed cells or may cause substantial nonselective inhibition of transformants and nontransformants alike, thus causing the selection technique to not be effective. It is proposed that selection with a growth inhibiting compound, such as bialaphos or glyphosate at concentrations below those that cause 100% inhibition followed by screening of growing tissue for expression of a screenable marker gene such as luciferase would allow one to recover transformants from cell or tissue types that are not amenable to selection alone. It is proposed that combinations of selection and screening may enable one to identify transformants in a wider variety of cell and tissue types. This may be efficiently achieved using a gene fusion between a selectable marker gene and a screenable marker gene, for example, between an NPTII gene and a GFP gene.

B. Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. A preferred growth regulator for such purposes is dicamba or 2,4-D. However, other growth regulators may be employed, including NAA, NAA+2,4-D or perhaps even picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soiless plant growth mix, and hardened, e.g., in an environmentally controlled chamber at about 85% relative humidity, 600 ppm CO₂, and 25-250 microeinsteins m⁻²s⁻¹ of light. Plants are preferably matured either in a growth chamber or greenhouse. Plants are regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants are preferably grown at about 19 to 28° C. After the regenerating plants have reached the stage of shoot and root development, they may be transferred to a greenhouse for further growth and testing.

Note, however, that seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10⁻⁵M abscisic acid and then transferred to growth regulator-free medium for germination.

Progeny may be recovered from transformed plants and tested for expression of the exogenous expressible gene by localized application of an appropriate substrate to plant parts such as leaves. In the case of bar transformed plants, it was found that transformed parental plants (R_{O}) and their progeny of any generation tested exhibited no bialaphos-related necrosis after localized application of the herbicide Basta to leaves, if there was functional PAT activity in the plants as assessed by an in vitro enzymatic assay. All PAT positive progeny tested contained bar, confirming that the presence of the enzyme and the resistance to bialaphos were associated with the transmission through the germline of the marker gene.

C. Characterization

To confirm the presence of the exogenous DNA or “transgene(s)” in the regenerating plants, a variety of assays may be preformed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

D. DNA Integration, RNA Expression and Inheritance

Genomic DNA may be isolated from callus cell lines or any plant parts to determine the presence of the exogenous gene through the use of techniques well known to those skilled in the art. Note, that intact sequences will not always be present, presumably due to rearrangement or deletion of sequences in the cell.

The presence of DNA elements introduced through the methods of this invention may be determined by polymerase chain reaction (PCR™). Using this technique discreet fragments of DNA are amplified and detected by gel electrophoresis. This type of analysis permits one to determine whether a gene is present in a stable transformant, but does not prove integration of the introduced gene into the host cell genome. It is typically the case, however, that DNA has been integrated into the genome of all transformants that demonstrate the presence of the gene through PCR™ analysis. In addition, it is not possible using PCR™ techniques to determine whether transformants have exogenous genes introduced into different sites in the genome, i.e., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR™, e.g., the presence of a gene.

Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

E. Gene Expression

While Southern blotting and PCR™ may be used to detect the gene(s) in question, they do not provide information as to whether the corresponding protein is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

IX. Definitions

Genetic Transformation: A process of introducing a DNA sequence or construct (e.g., a vector or expression cassette) into a cell or protoplast in which that exogenous DNA is incorporated into a chromosome or is capable of autonomous replication.

Expression: The combination of intracellular processes, including transcription and translation undergone by a coding DNA molecule such as a structural gene to produce a polypeptide.

Obtaining: When used in conjunction with a transgenic plant cell or transgenic plant, obtaining means either transforming a non-transgenic plant cell or plant to create the transgenic plant cell or plant, or planting transgenic plant seed to produce the transgenic plant cell or plant.

Promoter: A recognition site on a DNA sequence or group of DNA sequences that provides an expression control element for a structural gene and to which RNA polymerase specifically binds and initiates RNA synthesis (transcription) of that gene.

Regeneration: The process of growing a plant from a plant cell (e.g., plant protoplast, callus or explant).

Selected DNA: A DNA segment which one desires to introduce into a plant genome by genetic transformation.

Transformation construct: A chimeric DNA molecule which is designed for introduction into a host genome by genetic transformation. Preferred transformation constructs will comprise all of the genetic elements necessary to direct the expression of one or more exogenous genes. In particular embodiments of the instant invention, it may be desirable to introduce a transformation construct into a host cell in the form of an expression cassette.

Transformed cell: A cell the DNA complement of which has been altered by the introduction of an exogenous DNA molecule into that cell.

Transgene: A segment of DNA which has been incorporated into a host genome or is capable of autonomous replication in a host cell and is capable of causing the expression of one or more cellular products. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

Transgenic plant: A plant or progeny plant of any subsequent generation derived therefrom, wherein the DNA of the plant or progeny thereof contains an introduced exogenous DNA segment not originally present in a non-transgenic plant of the same strain. The transgenic plant may additionally contain sequences which are native to the plant being transformed, but wherein the “exogenous” gene has been altered in order to alter the level or pattern of expression of the gene.

Triterpene biosynthesis gene: A gene encoding a polypeptide that catalyzes one or more steps in the triterpene biosynthetic pathway.

Vector: A DNA molecule capable of replication in a host cell and/or to which another DNA segment can be operatively linked so as to bring about replication of the attached segment. A plasmid is an exemplary vector.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Development of an Inducible Cell Culture System for Functional Genomics Approaches to Identify Further Triterpene Saponin Biosynthetic Genes in Medicago Truncatula

The reactions of triterpene biosynthesis beyond the initial cyclization step catalyzed by β-AS are complex, and none of the enzymes involved in Medicago has been characterized at the molecular level. In order to use DNA micro- and/or macro-array experiments to discover these enzymes by genomics approaches, it was necessary to develop a system in which the saponin pathway can be rapidly and reproducibly induced from low basal levels.

Extraction and quantitation of the multiple M. truncatula triterpene saponins is not trivial (Huhman et al., 2002), and is therefore not the best assay method for determining expression of the triterpene pathway. It was thus decided to measure changes in transcript levels by RNA gel blot analysis, using the functionally confirmed Medicago β-AS, SE and SS as probes, in a series of studies designed to investigate conditions for inducing triterpene synthesis in M. truncatula root cell suspension cultures. Previous studies have shown effects of sucrose and mineral nutrients on saponin production in plant cell suspension cultures, but these effects were neither large nor rapid (Fulcheri et al., 1998; Akalezi et al., 1999).

Cell suspension cultures were initiated from roots of line A17, maintained in a modified Schenk and Hildebrandt medium, as described previously for alfalfa cultures (Dixon et al., 1981), and subcultured every 10-14 days. Six days after subculture, dark-grown M. truncatula A-17 root suspension cultures (75 ml batches) were treated with methyl jasmonate (MeJA, 500 μM), yeast elicitor (YE, 50 μg glucose equivalents ml⁻¹), salicylic acid (SA, 500 μM) or abscisic acid (ABA, 500 μM), harvested at various times after elicitation and frozen at −80° C. Control cells were treated with the same volume of distilled water. Thirty micrograms of M. truncatula RNA from elicited root cell suspension culture was separated by electrophoresis in a 1% agarose gel containing 0.66 M formaldehyde and then blotted onto a Hybond-N⁺ membrane (Amersham). The entire cDNA fragments of SS, SE1, SE2 and β-AS, and the M. truncatula cycloartenol synthase (NF015H10LF), phenylalanine ammonia-lyase (NF011C12ST) and chalcone synthase (NF044D07EC) EST clones were radiolabeled with [³²P] dCTP using a Ready-to-go DNA Labeling Beads (-dCTP) kit (Amersham) and used as probes.

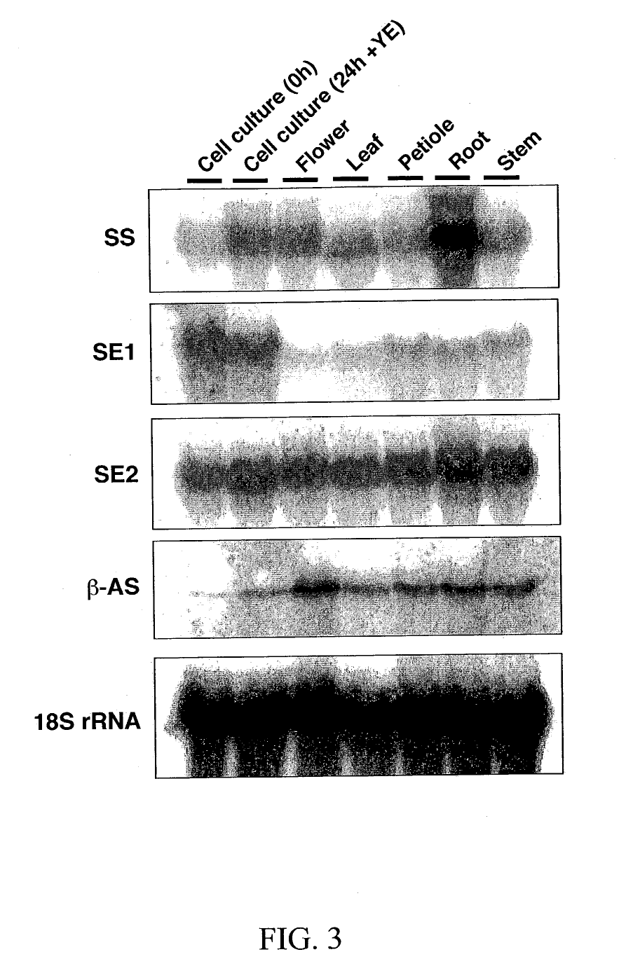

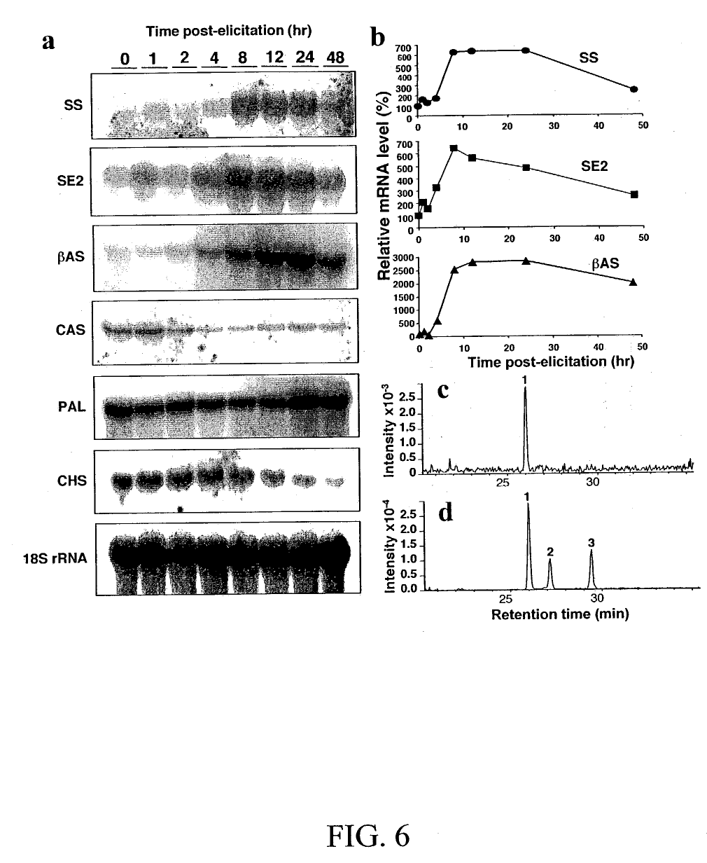

YE weakly induced SS, SE2 and β-AS transcripts, as previously shown in FIG. 3. Enhancement of β-AS transcript levels was 2- and 6-fold at 12 h post-elicitation with YE and SA respectively. β-AS transcripts were induced to a maximum level of 2.5-fold one hour after exposure to ABA. Strongest elicitation of β-AS transcripts was found with MeJA, which induced an increase of up to 30-fold by 8-24 h post-elicitation (FIG. 6A, B). SS transcripts were coordinately induced with β-AS transcripts in response to MeJA (FIGS. 6A, B). SE1 transcripts were not significantly induced by MeJA, whereas SE2 transcript induction closely followed that of β-AS (FIGS. 6A, B). In contrast, elicitation with MeJA caused a significant reduction in cycloartenol synthase transcript levels. The down-regulation of cycloartenol synthase transcripts following exposure to MeJA suggested preferential channeling of oxidosqualene from sterol synthesis to triterpene synthesis following elicitation.

Treatment of cell cultures with MeJA was confirmed to induce accumulation of triterpene saponins, as assessed by chemical extraction and analysis by LC/MS (FIGS. 6C, D). Saponin extracts were obtained using a solid phase extraction procedure as previously described (Oleszek, 1988; Oleszek et al., 1990). Six g fresh weight of cells were extracted in 80% methanol for 24 h. The extracts were concentrated under a nitrogen stream to yield an aqueous solution that was diluted to a final concentration of 35% methanol (v/v) and loaded onto a 35 ml, 10 g, C18 SPE extraction cartridge (Waters, Milford, Mass.). The SPE cartridge was washed with two column volumes each of HPLC grade water and 35% methanol. The saponins were eluted with two column volumes of 100% methanol. The methanol fraction was dried under vacuum, resuspended in methanol to a final concentration of ca. 400 ng μl⁻¹ and analyzed by gradient elution, reverse-phase HPLC with simultaneous on-line UV and mass selective detection (Huhman, et al., 2002).

Small amounts of hederagenin glycoside were present in unelicited cultures. These increased approximately 10-fold by 24 h post-elicitation, and glycosides of soyasapogenols B and E appeared. The latter compounds were not detected in the unelicited cultures (FIGS. 6 C, D).

MeJA has been suggested to be a signal molecule for the biosynthesis of phytoalexins (Gundlach et al., 1992). In Medicago species, the phytoalexins are isoflavonoids derived from the phenylpropanoid/flavonoid pathway (Dixon, 1999). To determine whether the phenylpropanoid pathway is co-induced with the triterpene biosynthetic pathway following exposure of cells to MeJA, membranes were probed with labeled M. truncatula phenylalanine ammonia-lyase (PAL) and chalcone synthase (CHS) cDNAs. PAL transcripts were only weakly induced by MeJA, with a maximum increase of only 1.5-fold at 24 h post-elicitation. More strikingly, CHS transcript levels decreased in parallel to the increase in β-AS mRNA (FIG. 6A).

### Example 2

### Use of Bioinformatic and DNA Array-based Approaches to Identify Novel Saponin Biosynthetic Genes in M. Truncatula—Approach #1

M. truncatula root cell suspension cultures produce low levels of triterpene saponins and have correspondingly low steady state levels of SS, SE and, particularly β-AS transcripts. In order to overcome this, conditions were determined by the inventors for rapid induction of triterpene biosynthesis in the cultures following exposure to MeJA. Jasmonates are important stress signaling molecules that elicit a wide range of secondary metabolites such as polyamines, coumaryl-conjugates, anthraquinones, naphthoquinones, polysaccharides, terpenoids, alkaloids and phenylpropanoids from different plant origins (Memelink et al., 2001). In Medicago cell suspension cultures, exposure to MeJA down-regulates the flavonoid branch of phenylpropanoid biosynthesis, as assessed by CHS steady state transcript levels, but induces the appearance of glycosides of the triterpenes hederagenin and soyasapogenols B and E. In contrast, exposure of the cells to yeast elicitor results in a strong induction of the phenylpropanoid pathway associated with accumulation of isoflavonoid phytoalexins, but with little effect on triterpene biosynthesis. The later enzymes of triterpene biosynthesis are believed to be primarily cytochrome P450s and glycosyl transferases, but none has yet been functionally characterized. These enzymes exist as large supergene families in plants (Chapple, 1998; Vogt and Jones, 2000), with approximately 250 members of the P450 family estimated from current M. truncatula EST information. Glycosyltransferase activity has been shown to correlate with saponin production in root cultures of Gypsophila paniculata (Herold and Henry, 2001), but specific triterpene glycosyltransferases remain to be characterized at the molecular level.

The ability to differentially up-regulate two major pathways of natural product metabolism, i.e., triterpenes and phenylpropanoids/flavonoids, in a cell culture system facilitates the design of DNA macro-and micro-array experiments for selection of candidate P450 and glycosyltransferase genes, which for example could be carried out using an EST collection (Bell et al., 2001). These can then be functionally characterized in yeast (P450s) or E. coli (GTs).

P450 and GT targets in MTGI were identified and annotated using the BLAST program (Altschul et al., 1997; ftp.ncbi.nih.gov/blast/executables/). The datasets that were used for searching MTGI were known P450 or GT proteins extracted from ATH1 (TIGR), Swiss-Prot and TrEMBL releases. Mining of the EST datasets indicated that Medicago truncatula appears to express approximately 249 distinct cytochrome P450 genes (Table 2). This number was computed from the number of Tentative Consensus (TC) sequences comprising contiguous overlapping EST clones plus the number of singletons in the libraries. The number of putative glycosyltransferase genes was likewise computed as approximately 286. Although many of the glycosyltransferase genes were annotated based on sequence similarity they are not functionally characterized, and annotations may be questionable for the glycosyltransferases in view of the close sequence alignment of known GTs with differing substrate specificities (Vogt and Jones, 2000), so all 286 were carried through to the next stage of analysis (Table 3).

The TIGR M. truncatula gene index contains EST sequence information from several different cDNA libraries from a range of different M. truncatula tissues and physiological treatments. Because β-AS is the first enzyme specific for triterpene saponin biosynthesis, one would predict that sequences encoding P450s or GTs involved in saponin biosynthesis would only be recovered from those cDNA libraries that also contained β-AS sequences and that, at first approximation, the higher the β-AS expression in a particular library, the higher the specific P450 and GT expression. The libraries were therefore “ordered” in relation to the number of β-AS ESTs recovered per 10,000 ESTs sequenced in any particular library. The most β-amyrin synthase ESTs were found in the cDNA library from Medicago leaves exposed to insect herbivory (Table 1). Therefore, an analysis was carried out of all the libraries for P450 and GT EST expression levels using clustering and self-organizing map algorithms to determine which. P450s and GTs had similar expression patterns to that of β-AS. Cluster analysis of the gene expression profiles was performed using the GENECLUSTER program (Tamayo et al., 1999) for self-organizing maps (SOM) and CLUSTER (Eisen et al., 1998) for hierarchical clustering. These results are given in Tables 4 and 5, which list the TC numbers of the P450 and GT clones whose expression patterns are similar to that of β-AS and which may therefore have involvement in triterpene saponin biosynthesis.

Fifty two cytochrome P450 clones and 75 GT clones with ESTs present in the insect herbivory library were spotted to duplicate membranes (as macroarrays with quadruplicate spots for each P450) and hybridized with ³²P-labeled RNA prepared from control and MeJA-treated M. truncatula cell suspension cultures. A number of these P450 and GT clones were strongly expressed in response to jasmonate but were not expressed in the control cultures. These are listed in Tables 6 and 7, and are strong candidates for having an involvement in triterpene saponin biosynthesis.

### Example 3

### Use of Bioinformatic and DNA Array-based Approaches to Identify Novel Saponin Biosynthetic Genes in M. Truncatula—Approach #2

One hundred and twenty eight putative cytochrome P450 (P450) and 164 putative glycosyltransferases (GT) clones from 36 Medicago truncatula EST libraries were spotted in duplicate and evaluated as representative for each TC by macroarray hybridization. cDNA inserts cloned into pBluescript were amplified by PCR of 2 μL of 150-μL resuspended plasmid DNA from overnight bacterial cultures using standard M13F and M13R primers. The quality of each PCR product was examined by gel electrophoresis.

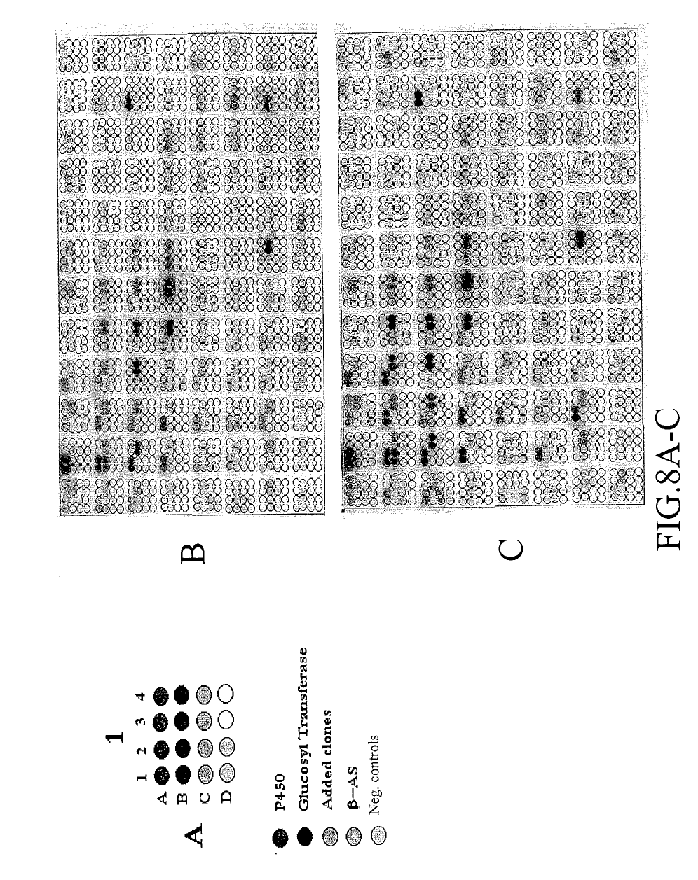

Approximately 100 ng of each PCR product was spotted in duplicate onto Hybond-N+ membranes (Amersham Pharmacia Biotech). Macroarray analysis was performed in triplicate using three separate RNA preparations, and hybridization was performed with 32P-labeled Medicago truncatula cell culture first strand cDNA probes. Single-stranded probes were synthesized from total RNA using SuperScript II reverse transcriptase (Invitrogen Life Technologies, Carlsbad, Calif.). The reaction mixture included 7 μL of RNA primer solution (3 μg of total RNA and 0.5 μg of oligo(dT)12-18 primer, annealed by heating to 70° C. for 10 min), 4 μL of 5×first strand buffer, 2 μL of 0.1 M dithiothreitol, 1 μL of dNTP mix (2.5 mM dATP, 2.5 mM dGTP, 2.5 mM dTTP, and 0.0625 mM dCTP), 5 μL of [−32P]dCTP (10 mCi mL 1), and 1 μL (200 units) of SuperScript II reverse transcriptase. Labeling was performed for 1 h at 42° C. Unincorporated [32P]dCTP was removed by passing the mixture through Sephadex G50-G150 columns. 32P incorporation was quantified via liquid scintillation counting. The final concentration of each probe was adjusted to 106 cpm mL 1 hybridization solution. The blots were prehybridized in Church buffer (1 mm EDTA, 0.5 M Na2HPO4, pH 7.2, and 7% SDS) at 65° C. for 2 h (Church and Gilbert, 1984) and then hybridized with 32P-labeled probe in 10 mL of Church buffer at 65° C. overnight. The blots were washed (Church and Gilbert, 1984), and the radioactive intensity of the spots on the macroarray filter was captured by a Phosphor Screen imaging system (Molecular Dynamics/Amersham Biosciences, Piscataway, N.J.). A typical result is shown in FIG. 8.

For data analysis, the signal intensities of the reference (0 hr exposure to MeJA) and test hybridization (24 hr following exposure to MeJA) were quantified using the software Arrayvision 6.0 (Imaging Research Inc., Haverhill, UK). The array organization consisted of 4×4 spots (Levell) and 8×12 spot groups (Level 2). The background was defined as the average of surrounding spot groups. Medicago truncatula β-amyrin synthase cDNA (Gene Bank Accession ID CAD23247) was used as a positive control and was spotted in duplicate in each 4×4 group. cDNAs of negative controls such as phosphinothricin acetyl transferase (GeneBank ID X17220), green fluorescent protein (AF078810), globin (NM_{—}000518), beta-glucuronidase (uidA; A00196), hygromycin B phosphotransferase (K01193), luciferase (X65316) and kanamycin/neomycin phosphotransferase (V00618) were randomly included in duplicate with each set of 4×4 spots. The induced expression level of a given clone-candidate was deduced from the ratio of the volume of the spot at 24 hr following exposure to MeJA and 0 hr. The volume was defined as the density value of each spot multiplied by its area. Density value is the average of all the pixels contained in the element. The values of ratios were extracted to an Excel sheet and subsequently analyzed.

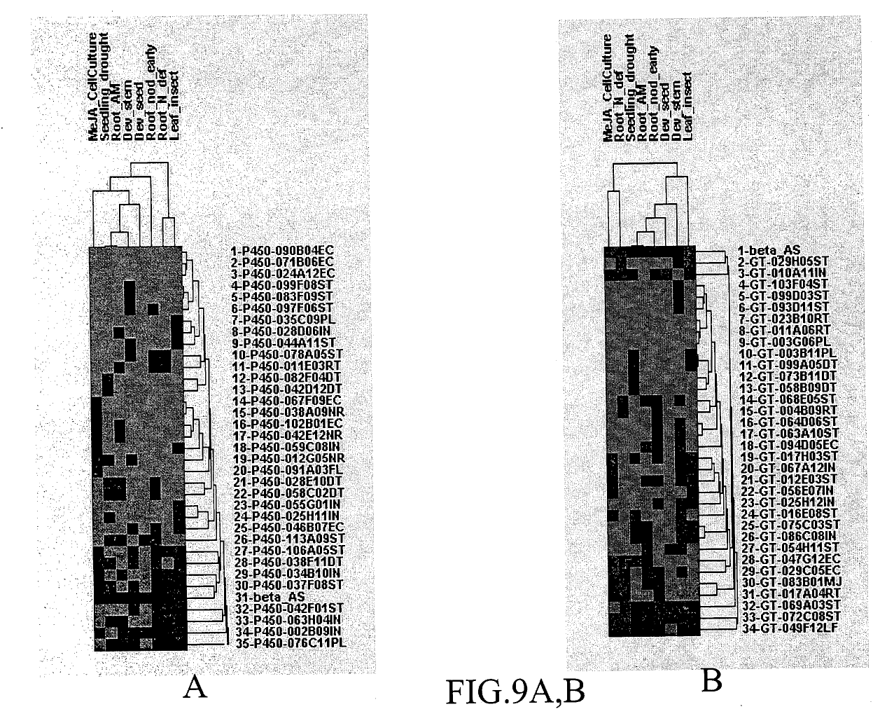

A second round macroarray experiments was performed spotting individual induced clones on a new template and the same analysis, as described above, was performed. The macroarray analysis, described above, revealed 38 P450 and 33 GT clones that were induced (at least 3 fold) in response to MeJA. These clones were further analyzed by mining a Noble Foundation local warehouse database (//bioinfo.noble.org) to determine which clones are coordinately expressed with Medicago truncatula β-amyrin synthase (Genbank accession CAD23247) in M. truncatula EST libraries. The sequence data obtained from the methyl jasmonate induced M. truncatula cell suspension culture library, which is not publicly available, was analyzed manually using the key word search and BLAST features. The obtained records were then analyzed in the local warehouse and TIGR database (www.tigr.org/tdb/tgi/mtgi/) to avoid redundancy. The EST count data were represented as % of frequency, which is defined as the EST counts divided by the dataset size (total number of clones in a given EST library); and the obtained ratio was multiplied by 100. M. truncatula β-amyrin synthase is expressed in the following cDNA libraries: germinating seed, insect-damaged leaf, developing stem, early-nodulated roots (1-4 days), nitrogen-starved roots, mycorrhiza inoculated root, drought-induced whole plants and methyl jasmonate induced cell suspension culture. Later, a cutoff was set to eliminate all induced P450 and GT clones from the macroarray experiment that have a % frequency higher that 0.05 (i.e., 5 EST counts per 10,000 clones) in the EST libraries where β-AS is not expressed at all. This analysis yielded 14 P450 (18 GT) candidate-clones that are coordinately expressed with β-AS (in at least 2 libraries out of 8) and do not exhibit strong expression in libraries where β-AS is not expressed. The remaining 10 P450 (7 GT) candidates are poorly co-expressed with β-AS (in less that 2 libraries) and do not show high expression in libraries where β-AS is not expressed. FIG. 9 shows the clustering analysis of the candidate clones.

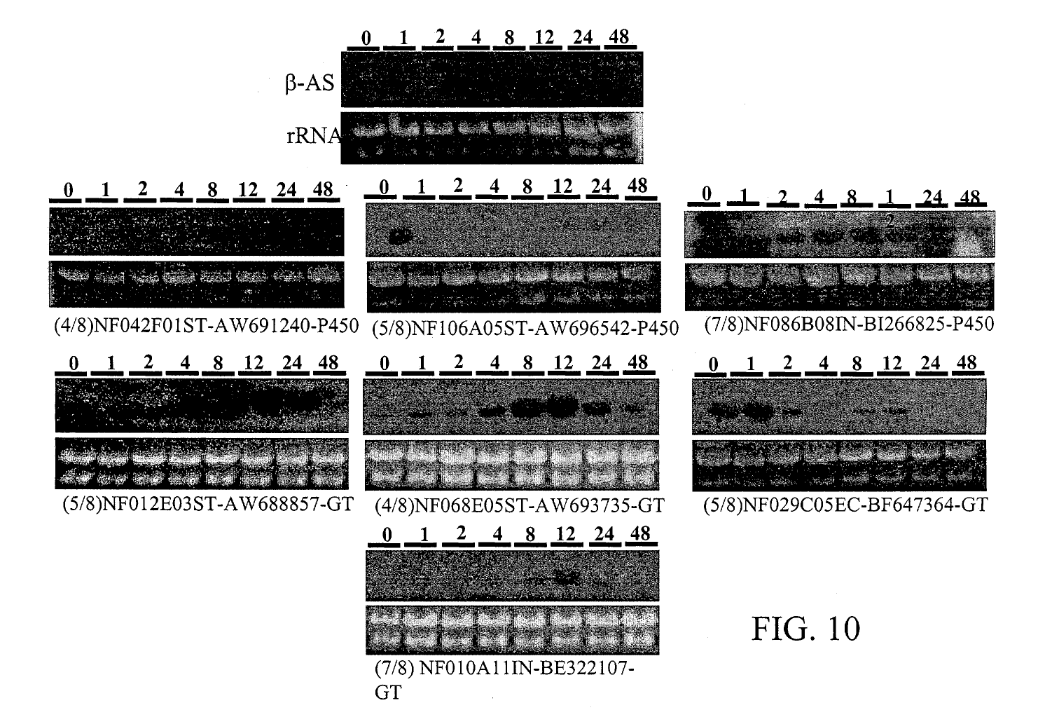

In order to further discriminate between the resulting P450 and GT clones, a kinetic study was performed of transcript levels following MeJA-induced elicitation. Gel preparation and Northern blotting were carried out by standard procedures (Sambrook et al., 2001). Twelve micrograms of M. truncatula RNA from a root cell suspension culture (time course for elicitation with MeJA) was electrophoresed through a denaturing gel before blotting to a Hybond-N+ (Amersham-Pharmacia Biotech) nylon membrane. Full-length cDNAs of the P450 and GT candidate clones were PCR-amplified from pBluescript (M13F and M13R) and labeled using the manufacturer's labeling procedure for nucleic acids (Amersham-Pharmacia Biotech). Hybridization was carried out overnight at 42 oC and the intensity of the signal was evaluated using ECL Direct Nucleic Acid Labeling and Detection Systems (Amersham-Pharmacia Biotech). FIG. 10 shows a typical result with selection of the clones.

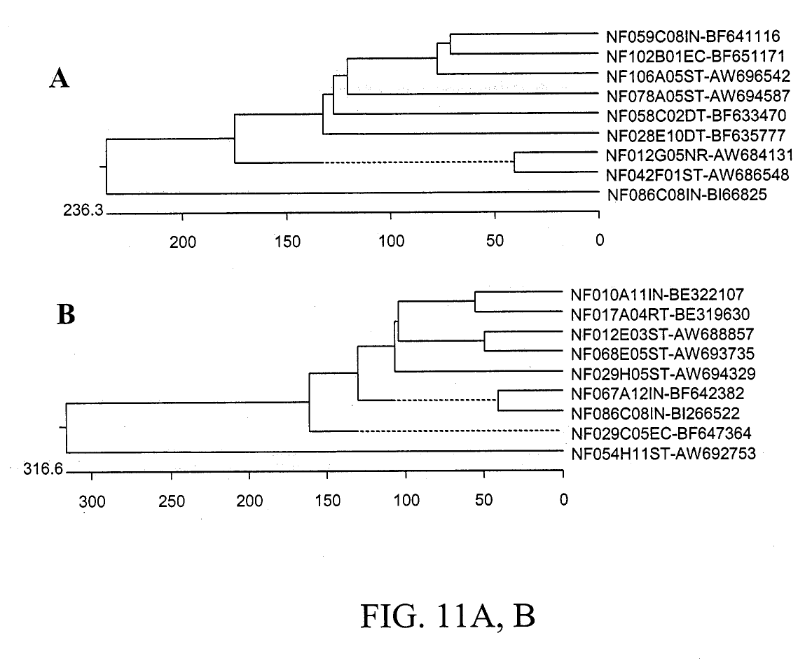

Based on all the above bioinformatic and transcript expression analysis, 7 P450 and 7GT clones were identified as being tightly associated with the triterpene saponin pathway and existed as full length clones in the Noble Foundation EST collection. Their nucleotide sequences are given as SEQ ID NO:18-31. Phylogenetic analyses of the top 9 triterpene pathway candidate P450 and GT clones is shown in FIG. 11. One embodiment of the current invention therefore provides these nucleic acid sequences, polypeptides encoded thereby, other nucleic acids encoding these polypeptides, and vectors comprising such nucleic acids, including transformation constructs comprising such nucleic acids operably linked to a heterologous regulatory region. Still further provided is a method of modifying, or increasing, saponin biosynthesis in a plant comprising introducing one or more than one of any of the aforementioned coding sequences into the plant.

### Example 4

### Identification of Early Triterpene Pathway Genes in M. Truncatula by Mining EST Datasets

Candidate ESTs for the first steps of the saponin biosynthetic pathway in M. truncatula were identified by mining publicly available EST datasets representing cDNA libraries from a variety of different organs and biotic/abiotic treatments (Table 1). To obtain full-length mRNA sequences for the putative SS, SE and β-AS genes, EST clones were retrieved and analyzed that were found in cDNA libraries from M. truncatula roots, nodulated roots, stems, cell suspension cultures and leaves, and from some of the above tissues following treatments such as insect damage, elicitation with yeast extract, drought, or phosphate starvation.

In the TIGR M. truncatula Gene Index (MtGI) database (Quackenbush et al., 2000; www.tigr.org/tdb/mtgi/), the putative SS ESTs were clustered into one tentative consensus, whereas putative SE and β-AS ESTs were each clustered into three tentative consensuses (Table 1).

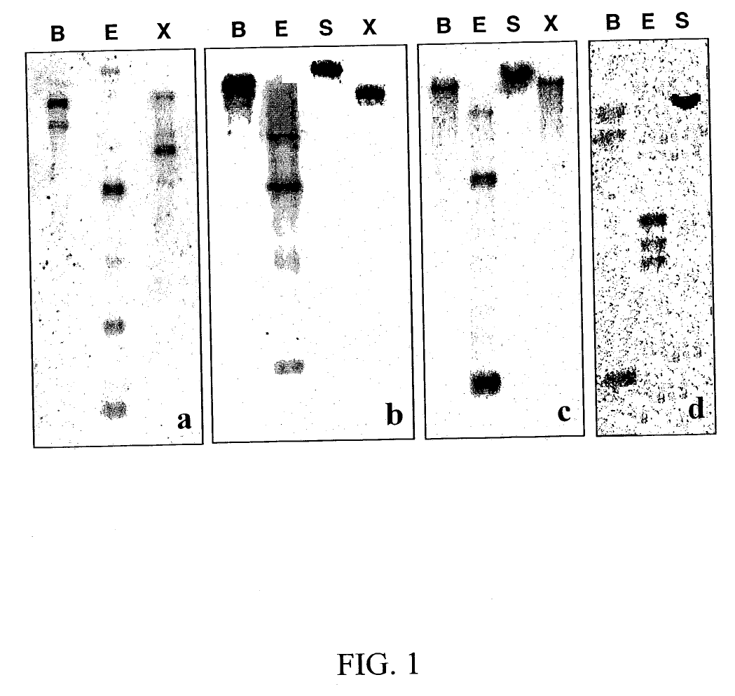

DNA was extracted from M. truncatula plants by standard methods (Sambrook et al., 2001). Southern blotting and hybridization were carried out as described previously (Church and Gilbert, 1984). The SS, SE1, SE2 and β-AS probes were amplified as complete ORFs from the EST clones NF066G09IN, NF065G06EC (SE1), NF102D09LF (SE2), and NF051E06IN, respectively. Two SE genes, SE1 and SE2, were each present in a single copy in the M. truncatula genome, as shown by DNA gel blot analysis in FIGS. 1B, C. Neither SE1 nor SE2 had restriction sites for BamHI, SalI or XbaI. EcoRI cuts once in SE2, but did not cut SE1; thus, the common major band in the EcoRI lanes in FIGS. 1B and 1C was likely due to cross hybridization with the other SE gene. β-AS and SS were both present in two copies in M. truncatula (FIGS. 1A, D).

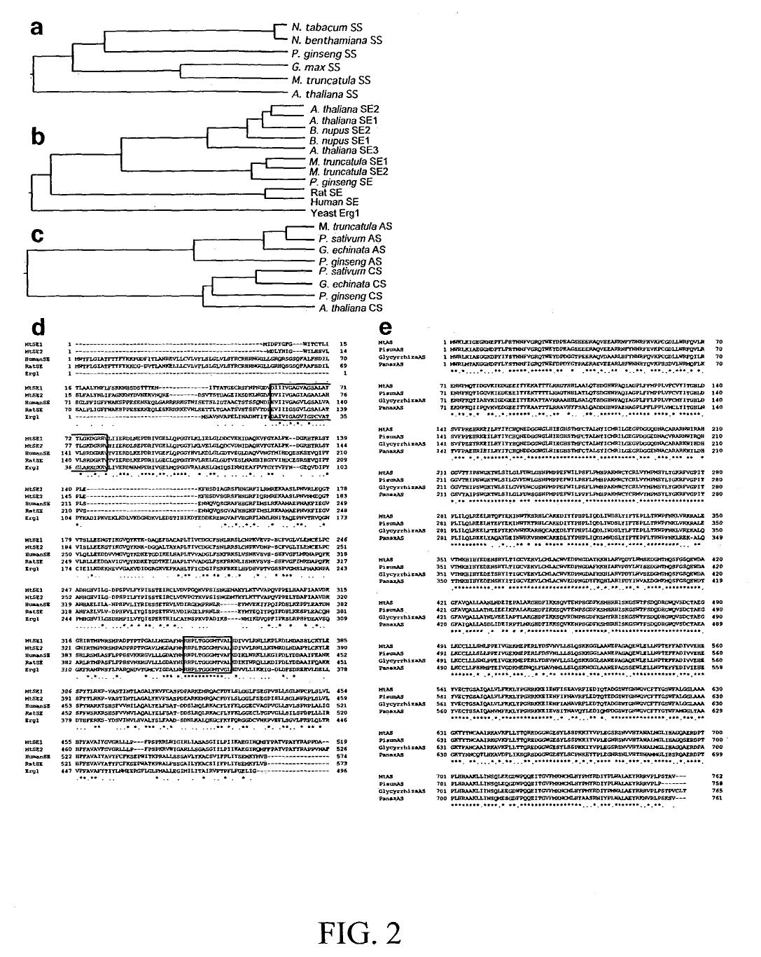

The dendrogram in FIG. 2A displays the relatedness of several reported plant SS proteins. M. truncatula (EST NF066G09IN, GenBank Accession # BF642230) and soybean (G. max) SS proteins were closely related, as would be expected since both species belong to the Fabaceae. M. truncatula SE1 (GenBank Accession # BF646034) and SE2 (GenBank Accession # BF646034) proteins are more closely related to Panax ginseng putative SE, with 77.1 and 74.4% sequence identity, respectively, than to Arabidopsis and Brassica SEs (FIG. 2B). The two distinct types of plant OCS, cycloartenol synthase and β-AS, exhibited a relatively high level of amino acid sequence identity, even though their reaction products were quite distinct (Kushiro et al., 1998; Hayashi et al., 2000). Alignment of known plant cycloartenol synthase and β-AS proteins indicated that the putative M. truncatula β-AS (GenBank Accession # BF640954) falls into the β-amyrin synthase group (FIG. 2C). M. truncatula β-AS protein was closely related to pea (Pisum sativum) β-AS with 94.7% sequence identity (FIG. 2C).

FIG. 2D shows amino acid sequence alignments of M. truncatula SE 1 and SE 2 with the enzymes from human, rat, and yeast. There was a high degree of sequence conservation in certain key regions, particularly around the squalene and FAD binding domains. The M. truncatula enzymes were more similar to the mammalian enzymes than to the enzyme from yeast. M. truncatula putative β-AS showed a high degree of sequence identity to the β-AS enzymes of pea, licorice and ginseng (FIG. 2E).

Table 1. M. truncatula EST clones and tentative consensus (TC) sequences annotated as squalene synthase (SS), squalene epoxidase (SE) and β-amyrin synthase (β-AS). Data are from cDNA libraries from a number of different tissue sources sequenced at the Samuel Roberts Noble Foundation. TC numbers correspond to the TIGR M. truncatula Gene Index (MtGI) at (www.tigr.org/tdb/mtgi/). TC sequences were assembled from ESTs, and may represent full-length transcripts. TC annotations contain information on the source library and abundance of ESTs. The tissue sources of the cDNA libraries were: a, root (6,593); b, stem (10,314); c, developing leaf (7,831); d, phosphate-starved leaf (9,034); e, drought induced whole plants (8,416); f, elicited cell culture (8,926); g, insect damaged leaf (9,921); h, developing flower (3,404); i, nodulated root (29,721); j, germinating seed (451). The numbers in brackets refer to the total number of ESTs sequenced in each library as of December 2001.

| | TC number | | | | | | | | | | |
|---|---|---|---|---|---|---|---|---|---|---|---|
| Gene | (no. of | Number of ESTs per library | | | | | | | | | |
| name | ESTs/TC) | A | b | c | d | e | f | g | h | i | j |
| Squalene | TC35874 | 3 | 2 | 0 | 0 | 1 | 2 | 4 | 0 | 0 | 0 |
| synthase | (12) | | | | | | | | | | |
| (SS) | | | | | | | | | | | |
| Squalene | TC28416 (7) | 0 | 0 | 1 | 1 | 0 | 1 | 2 | 1 | 1 | 0 |
| epoxidase | TC29021 (3) | 0 | 0 | 1 | 0 | 0 | 0 | 2 | 00 | 0 | 0 |
| (SE) | TC37711 (2) | 0 | 0 | 0 | 0 | 0 | 2 | 0 | | 0 | 0 |
| β-amyrin | TC28833 (3) | 0 | 0 | 0 | 0 | 1 | 0 | 0 | 0 | 0 | 2 |
| synthase | TC28834 (2) | 0 | 1 | 1 | 0 | 0 | 0 | 0 | 0 | 0 | 0 |
| (β-AS) | TC28878 (8) | 0 | 1 | 1 | 0 | 1 | 0 | 5 | 1 | 1 | 1 |

### Example 5

### Tissue Specific Expression of Early Saponin Pathway Genes in M. Truncatula

Tissue specificity of putative saponin biosynthetic enzyme transcripts was first assessed in silico from analysis of EST occurrence in the various cDNA libraries using the data available in the TIGR M. truncatula Gene Index database (Table 1). Putative triterpene pathway genes appeared to be expressed at a higher level in insect damaged leaves than in control leaves based on relative EST abundance (Table 1). Three SS full-length clones (including the one functionally characterized below) and one truncated clone were found among the 9,921 clones sequenced from the insect damaged leaf library, but only one truncated SE clone (NF026F08IN) was found. Two full-length and two truncated β-AS clones were found, including the one functionally characterized below. The highest abundance was five ESTs for β-AS from the insect damaged leaf library.

The essential features of the tissue specificity were confirmed by RNA gel blot analysis, in several cases using RNA samples from the original preparations used for cDNA library construction (FIG. 3). Medicago truncatula Gaerth ‘Jemalong’ (line A17) plants were grown under greenhouse conditions in 11 cm diameter pots in Metro-mix 250 or 350 (Scott, Marysville, Ohio, USA), nine plants per pot with an 18 h light/25° C. and 6 h dark/22° C. photoperiod. Thirty micrograms of M. truncatula RNA was separated by electrophoresis in a 1% agarose gel containing 0.66 M formaldehyde and then blotted onto a Hybond-N⁺ membrane (Amersham). The entire cDNA fragments of SS, SE1, SE2 and β-AS were radiolabeled with [³²P] dCTP using a Ready-to-go DNA Labeling Beads (-dCTP) kit (Amersham) and used as probes. Putative SS transcripts were abundant in roots, whereas flower, leaf, petiole, cell culture and stem showed a lower level of expression. The tissue distribution of M. truncatula SE1 transcripts showed that this gene is expressed weakly in petiole, root and stem, but only traces of the transcript are present in flowers and leaves. Exposure of M. truncatula cell suspension cultures to yeast elicitor for 24 h resulted in an enhancement of SS, but not SE1, transcripts. SE2 transcripts were expressed at higher levels than SE1 transcripts in all the tissues examined, with highest levels in root and stem and evidence of weak induction in cell cultures by yeast elicitor. Putative β-AS transcripts were most highly expressed in root, stem, flower, and petiole, and were induced from a very low basal level in yeast elicited cell cultures.

### Example 6

### Functional Characterization of M. Truncatula Squalene Synthase

SS catalyzes the reductive dimerization of two molecules of farnesyl diphosphate (FPP) in a two-step reaction to produce squalene. This reaction is believed to proceed via head-to-head coupling of two FPP molecules to form squalene via a stable cyclopropylcarbinyl diphosphate intermediate (Pandit et al., 2000). Functional expression of the M. truncatula putative SS cDNA in E. coli BL21 was accomplished by cloning of the coding sequence into the expression vector pET-15b after introducing NcoI and BamHI sites.

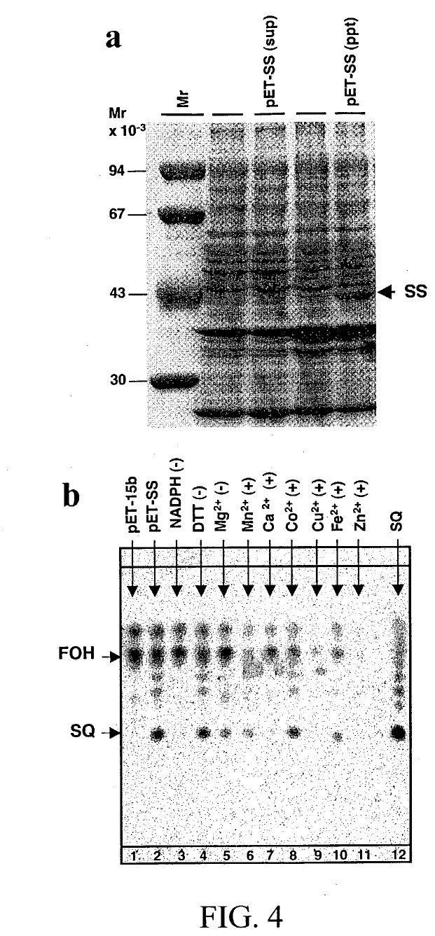

Expression of M. truncatula EST clone NF066G09IN was performed by amplification of the open reading frame from pBluescript II SK+ (Stratagene, La Jolla, Calif.) with introduction of NcoI and BamHI sites (5′-CCATGCCATGGGAAGTATAAAAGCGATTTTGAAGAATC-3′ (SEQ ID NO:8) for the upstream primer and 5′-CGGGATCCTTAGTTATTGTGACGATTGGCAGAGAG-3′ (SEQ ID NO:9) for the downstream primer). The PCR product was purified, ligated into pGEMTeasy vector (Promega, Madison, Wis., USA), sequenced, excised and re-cloned between the NcoI and BamHI sites of the pET 15b expression vector (Novagen, Madison, Wis., USA). E. coli BL21 (DE3, pLyS) cells harboring the expression construct were grown to an OD₆₀₀ of 0.6, and expression was induced by addition of isopropyl 1-thio-β-D-galactopyranoside (IPTG) to a final concentration of 0.5 mM, with further incubation for 3 h. Cell lysates were prepared and the crude extract used for protein gel blot and enzyme assay. SDS PAGE analysis of total proteins showed that a 43 kDa band, corresponding to the predicted size of the recombinant protein, appeared in extracts from IPTG induced E. coli, but not in cultures harboring an empty pET-15b vector (FIG. 4A).

For assay of squalene synthase, the reaction mixture contained, in a total volume of 100 μl, 11.4 μM [1,2-¹⁴C]-FPP (125 nCi; American Radiolabeled Chemicals, St. Louis, Mo.), 3 mM NADPH, 5 mM MgCl₂, 0.1 mM dithiothreitol, 100 mM KF, 50 mM Tris-HCl (pH 7.6), and enzyme (70 μg of protein). The reaction mixture was incubated at 30° C. for 1 h and the reaction stopped by addition of 100 μl ethyl acetate. Lipids were extracted with ethyl acetate and 10 μl subjected to TLC on silica gel reverse phase plates (RP-18) (J.T. Baker, Phillipsburg, N.J.). The plates were developed with acetone:water (19:1, v/v). After development, plates were exposed and analyzed with a bio-image analyzer (Molecular Dynamics, Sunnyvale, Calif.). No ¹⁴C-squalene product was formed in extracts of E. coli transformed with the empty vector (FIG. 4B). In contrast, extracts from E. coli transformed with pET15b containing recombinant SS (pET-SS), in spite of the very small proportion of soluble recombinant enzyme, catalyzed formation of a labeled product that co-migrated with authentic squalene. When NADPH was omitted, no squalene product was observed. A strong reduction in squalene formation was also observed on omitting Mg²⁺ from the incubation mixture, the residual activity perhaps being supported by endogenous Mg²⁺.

The M. truncatula SS reaction was further characterized by substituting Mg²⁺ in the incubation mixture with other divalent cations. Mn²⁺, Co²⁺ and Fe²⁺ could substitute for Mg²⁺ as cofactors in this reaction, whereas Ca²⁺, Cu²⁺ or Zn²⁺ could not (FIG. 4B).

Arabidopsis thaliana SS has been functionally expressed, and shown to produce squalene in the presence of Mg²⁺ and NADPH, and dehydrosqualene in the presence of Mn²⁺ but absence of NADPH (Nakashima et al., 1995). The M. truncatula squalene synthase could use Mn²⁺ or Mg²⁺ equally well as co-factors for squalene formation in the presence of NADPH. Interestingly, the intact full length Arabidopsis SS1 cannot complement a yeast SS mutant, even though the yeast cells expressing the Arabidopsis enzyme contain detectable SS activity when assayed in vitro. This has been shown to be due to a requirement for a specific C-terminal portion of the yeast SS for metabolic channeling of squalene through the yeast sterol pathway (Kribii et al., 1997). This was an interesting feature from the point of view of the organization of potential metabolic complexes necessary for channeling of squalene into either the triterpene or the sterol pathway in plants.

### Example 7

### Functional Characterization of M. Truncatula Squalene Epoxidase

SE catalyzes the insertion of an oxygen atom across a carbon-carbon double bond to form an epoxide in a reaction more typical of P450-type reactions. Squalene monoxygenases have been cloned and functionally characterized from yeast, rat and human (Jandrositz et al., 1991; Sakakibara et al., 1995; Laden et al., 2000), but not from plants. SE, encoded by the ERG1 gene in yeast, is a key enzyme in the sterol biosynthetic pathway. The KLN1 strain of yeast (MATα, erg1::URA3, leu2, ura3, trp1) used here for the functional characterization of putative M. truncatula SE, is an obligate ergosterol auxotroph; disruption of ERG1 is lethal, unless ergosterol is supplied to cells growing under anaerobic conditions (Landl et al., 1996).

To functionally characterize the two putative M. truncatula squalene epoxidases, the SE1 and SE2 coding sequences, with 47 amino acids truncated from the N-terminus of SE1, and 52 amino acids truncated from the N-terminus of SE2, and the ERG1 ORF as a positive control, were cloned into the pWV3 vector (gift from Dr. Wayne Versaw, Noble Foundation), containing the LEU2 selectable marker, under control of the constitutive pADH1 promoter. The N-terminal truncation sites were chosen by comparison with the yeast protein, which has a short N-terminus compared to plant or mammalian SE (FIG. 2D). Functional identification of putative squalene epoxidases encoded by M. truncatula EST clones NF065G06EC (SE1) and NF102D09LF (SE2), was achieved by heterologous expression in the Erg1 knockout yeast mutant KLN (MATα, erg1::URA3, leu2, ura3, trp) (Landl et al., 1996) (gift of Drs. R. Leber and F. Turnowsky, Institute of Molecular Biology, Graz University, Austria).

The PCR fragments with introduced BamHI and XhoI sites were amplified with the following primers: for the pWV3-SE1 construct, 5′-CGCGGATCCATGATAGACCCCTACGGTTTCGGGTGG-3′ (SEQ ID NO:10) for upstream and 5′-CCGCTCGAGTTATGCATCTGGAGGAGCTCTATAAT-3′ (SEQ ID NO:11) for downstream; for the pWV3-Δ47SE1 construct, 5′-CGCGGATCCATGTCTTTTAATCCCAACGGCGATGTTG-3′ (SEQ ID NO:12) for upstream; for the pWV3-SE2 construct, 5′-CGCGGATCCATGGATCTATACAATATCGGTTGGAATTTA-3′ (SEQ ID NO:13) for upstream and 5′-CCGCTCGAGTCAAAATGCATTTACCGGGGGAGCTC-3′ (SEQ ID NO:14) for downstream; for the pWV3-Δ52SE2 construct, 5′-CGCGGATCCATGTCGGACAAACTTAACGGTGATGCTG-3′ (SEQ ID NO:15) for upstream. For amplification of the yeast Erg1 sequence, 5′-CGGGATCCATGTCTGCTGTTAACGTTGCACCTGAATTG-3′ (SEQ ID NO:16) was used for the upstream primer and 5′-CCGCTCGAGTTAACCAATCAACTCACCAAACAAAAATGGG-3′ (SEQ ID NO:17) for downstream. The PCR products were purified, subcloned into pGEMTeasy vector, sequenced, excised and re-cloned between the BamHI and XhoI sites of the pWV3 yeast expression vector. The SE1 and SE2 ORFs, SE1 with 47 amino acids truncated from the N-terminus, SE2 with 52 amino acids truncated from the N-terminus, and the Erg1 ORF as a positive control, were under control of the constitutive ADH1 promoter, and the pWV3 vector contained the Leu2 selectable marker for yeast expression. Anaerobic conditions were achieved by culturing the yeast strains in an Anaerocult A chamber (VWR Scientific Products, Atlanta, Ga.). Ergosterol (final concentration 20 μg ml⁻¹) was dissolved in Tween 80/ethanol (1:1, v/v), with a final Tween 80 concentration of 0.5% (v/v) in the medium.

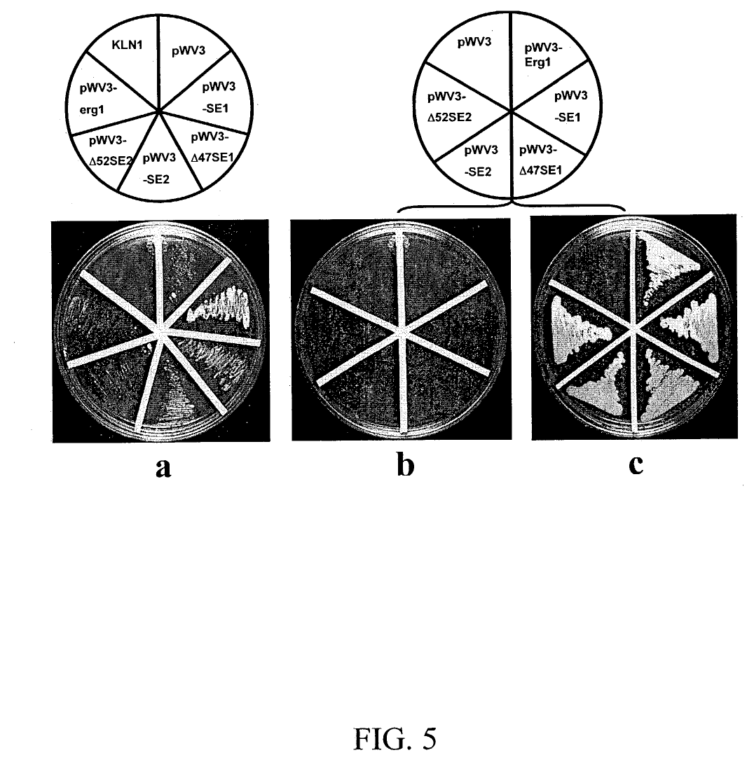

Selection of transformants for the Leu+ phenotype was made in SD medium supplied with ergosterol and tryptophan under anaerobic conditions (FIG. 5A). As expected, KLN1 did not grow because the medium was deprived of Leu (FIG. 5A) (Landl et al., 1996). When plated in YPD (or SD+trp) medium without ergosterol under anaerobic conditions, the transformants were not viable (FIG. 5B), whereas under aerobic conditions they exhibited strong growth (FIG. 5C). pWV3 transformants were not able to grow under either condition, showing that the SE or ERG1 (positive control) inserts contributed to this growth. Thus, the growth of the transformants is oxygen-dependent, as is the SE reaction. These data show that the ergosterol biosynthetic pathway in the yeast erg1 knockout could be reconstituted by heterologous complementation with M. truncatula SE with or without truncation of the N-terminus.

The fact that M. truncatula possesses two isoforms of squalene epoxidase, SE1 and SE2, raises the question of whether these may have different biochemical functions in relation to triterpene and sterol biosynthesis. This idea is indirectly supported by the co-induction of SE2, but not SE1, with β-AS in MeJA-treated cell cultures, as shown below.

Although plant genes with sequence similarity to mammalian SE have been described in the literature, the present report is believed by the inventors to be the first functional characterization of a plant SE. The two isoforms of M. truncatula squalene epoxidase, SE1 and SE2, share 82.1% amino acid identity. Both M. truncatula SEs could complement the ergosterol biosynthetic pathway in the Erg1 knockout yeast strain KLN1. This is interesting in view of the failure of Arabidopsis SS to correctly couple with the sterol biosynthetic machinery in yeast (Kribii et al., 1997), and the complexity of the mammalian squalene epoxidase reaction which requires, in addition to NADPH cytochrome P450 reductase, a specific lipid transfer protein for transfer of squalene to the enzyme (Shibata et al., 2001). This also suggests that, in spite of the differential induction of the two Medicago SEs in planta, with its implications for differential function, both forms might be able to participate in sterol biosynthesis in plant cells, as in the heterologous yeast system.

### Example 8

### Functional Characterization of M. Truncatula β-amyrin Synthase

EST clone NF051E06IN contained an apparent full-length oxidosqualene cyclase (OSC) gene in pBluescript SK⁻ vector. The plasmid was digested with NotI, XhoI and ScaI, to release the 2.8 kb insert with NotI and XhoI termini (ScaI was included to cut the 2.9 kb vector into 1.1 kb and 1.8 kb fragments, facilitating purification). The insert was subcloned into the yeast expression vector pRS426GalR that contains the URA3 selectable marker, the 2μ origin of replication, and Gal promoter. This high copy expression construct was named pRX10.2, and was transformed into yeast lanosterol synthase mutant SMY8 (MATa erg7::HIS3 hem1::TRP1 ura3-52 trp1-Δ63 leu2-3,112 his3-Δ200 ade2 Gal⁺). The transformants were selected on synthetic complete medium (containing 2% dextrose) lacking uracil and supplemented with heme (13 μg ml⁻¹), ergosterol (20 μg ml⁻¹) and Tween-80 (5 μl ml⁻¹). SMY8 harboring empty vector pRS426Gal was used as negative control in the following assay.

A 5-ml yeast culture was induced with 2% (w/v) galactose and grown to saturation. The harvested yeast cells were resuspended in 200 mM sodium phosphate buffer (pH 6.4), lysed by vortexing with glass beads, and incubated with 1 mg ml⁻¹ oxidosqualene and 0.1% Tween-80. The reaction was incubated at room temperature for 24 h and quenched with 4 volumes of ethanol. After centrifugation, the supernatant was transferred into a glass tube, and the cell debris was extracted with two further volumes of ethanol. The combined ethanol extract was dried under a nitrogen stream, redissolved in ethyl acetate and filtered through a small silica gel plug to remove cell debris and some polar components. The crude extract was derivatized to form trimethylsilyl (TMS) ethers by treatment with 50 μl of bis(trimethylsilyl)trifluoroacetamide-pyridine (1:1, v/v) at 40° C. for 2 h and was analyzed by GC-FID and GC-MS, with epicoprostanol (an unnatural C-30 sterol) TMS ether as internal standard and authentic β-amyrin TMS ether as external standard. Co-injection of crude product(s) and β-amyrin standard was performed on GC-MS.

GC analysis employed a Hewlett-Packard 6890 system equipped with a Rtx-5 capillary column (Restek, 30 m×0.25 mm i.d., 0.10 μm d_{f}). A 5 μl aliquot was injected at 280° C. with a split ratio of 40:1, helium flow was at 20 cm s⁻¹, and the following temperature program was applied: 100° C. for 2 min, rising to 280° C. at 20° C. min⁻¹, holding at 280° C. for 30 min. The flame ionization detector was at 280° C. GC-MS was performed on a Hewlett-Packard 5890A instrument equipped with a DB-5ms column (J&W, 60 m×0.25 mm i.d., 0.10 μm d_{f}). Separation was achieved with splitless injection (1 min delay) at 200° C., helium flow at 30 cm s⁻¹ (1 ml min⁻¹) and the identical temperature program as above. Mass spectra (m/z 35 to 500) were obtained on a ZAB-HF reverse-geometry double-focusing instrument at 70 eV with an electron-impact ion source (200° C.). The accelerating voltage was 8 kV and the resolution was 1000 (10% valley).

A 300 mL SMY8[RX10.2] yeast culture was processed similarly to obtain enough enzymatic product(s) for NMR analysis. The ethanolic supernatant of the in vitro catalytic reaction was evaporated to dryness and redissolved in ethyl acetate. The crude mixture was filtered through a silica plug and then separated by silica gel column chromatography to remove excess oxidosqualene substrate, exogenous ergosterol and fatty acids. Polycyclic triterpene alcohols co-migrate with β-amyrin on TLC, and fractions with material in this region were pooled and analyzed by ¹H NMR and GC-MS, which showed β-amyrin uncontaminated by other triterpene alcohol isomers (detection limit 2%). NMR spectra of free sterols were obtained on a Bruker AMX500 spectrometer (500 MHz for ¹H) at 25° C. in CDCl₃ solution and referenced to internal tetramethylsilane.

The enzyme encoded by EST NF051E06IN when expressed in yeast, cyclized oxidosqualene to form product(s) that comigrated with β-amyrin on TLC, whereas the yeast strain SMY8 harboring the empty vector did not form this compound(s). The GC relative retention time (Rt) of the cyclization product TMS ether was identical to that of authentic β-amyrin TMS ether (Rt=1.23, relative to epicoprostanol TMS ether). The mass spectra (MS) of the enzymatic product, β-amyrin standard and their coinjection agreed with each other. (EI-MS): (TMS ether) m/z=498 [M]⁺ (6%), 483 [M-CH₃]⁺ (3%), 408 [M-Me₃SiOH]⁺ (2%), 393 [M-Me₃SiOH—CH₃]⁺ (3%), 218 (C-ring fragment, 100%), 203 [m/z 218-CH₃]⁺ (39%). NMR data further confirmed the identification of β-amyrin. Key ¹H NMR signals of the authentic sample matched those of the NF051E06IN product to ±0.001 ppm (500 MHz, CDCl₃, tetramethylsilane as internal standard): δ5.184 (t, 3.5 Hz, 1H, H-12), 3.223 (ddd, 11.2, 6.0, 4.7 Hz, 1H, H-3), 1.135 (d, 1.0 Hz, 3H, H-27), 0.998 (s, 3H, H-23), 0.968 (s, 3H, H-26), 0.938 (s, 3H, H-25), 0.872 (s, 6H, H-29, H-30), 0.832 (s, 3H, H-28), 0.792 (s, 3H, H-24), 0.742 (d, 11.7, 1.9 Hz, 1H, H-5). The observed MS and NMR data agreed with literature values for β-amyrin (Segura et al., 2000). A 290 ml yeast culture produced 1.7 mg of β-amyrin (>98% pure) from 14 mg of oxidosqualene substrate.

The formation of β-amyrin by cyclization of 2,3-oxidosqualene is a complex reaction believed to occur via the “chair-chair-chair” conformation of the substrate. The OSCs lanosterol and cycloartenol synthase have been extensively studied in mammals and yeast (Corey et al., 1993; Corey et al., 1994; Corey et al., 1996; Abe and Prestwich, 1995; Morita et al., 1997). Recently, cDNAs encoding three proteins from Arabidopsis thaliana with 49 to 59% identity to cycloartenol synthase were functionally expressed. The products of one of these enzymes consisted of a mixture of lupeol, β-amyrin and α-amyrin (15:55:30) (Husselstein-Muller et al., 2001), whereas M. truncatula β-AS catalyzed the formation of β-amyrin alone, with no minor products, as also observed for the Panax enzyme (Kushiro et al., 1998).

### Example 9

### Characterization of the Upstream Regulatory Sequence of Medicago Sativa SE2

A genomic library of the alfalfa (Medicago sativa) cultivar Apollo in the γ Fix II system (Stratagene) was screened with a probe derived from the M. truncatula squalene epoxidase 2 cDNA described above. The transfer membrane was hybridized in 0.5 M Na₂HPO₄ buffer pH 7.2, 7% SDS at 63° C. overnight. The membrane was pre-washed in 40 mM Na₂HPO₄ buffer pH 7.2, 5% SDS for 20 minutes, then washed twice in 40 mM Na₂HPO₄ buffer, pH 7.2, 5% SDS at 63° C. for 30 minutes each and twice in 40 mM Na₂HPO₄ buffer, pH 7.2, 1% SDS at 63° C. for 30 minutes each. Positive clones from the first screening were subjected to two additional rounds of screening. DNA from the purified phage clones was analyzed by restriction enzyme digestion and DNA gel blot hybridization. The phage DNA was then digested with NotI and subcloned into pBluescript II KS. The DNA was sequenced by a transposon strategy following the manufacturer's instructions (Invitrogen). The sequence of the open reading frame was 97% identical at the amino acid level to that of M. truncatula SE2, and 83% identical to that of M. truncatula SE1, suggesting that the gene encodes the SE form most likely to be involved in triterpene biosynthesis. The sequence of the upstream promoter region, which was shown to be functional in Medicago by transient expression studies with the β-glucuronidase reporter gene, is given in SEQ ID NO:1.

TABLE 2
Cytochrome P450 ESTs (TCs and singletons) from Medicago truncatula as first round candidates for involvement in
triterpene saponin biosynthesis. Numbers refer to TIGR Medicago Gene Index TC or singleton numbers.

| TC/EST | Annotation | Best hit in the dataset | Bitscore | Evalue |
|---|---|---|---|---|
| TC28294 | Cytochrome P450 like_TBP (EC 1.14.14.1) (TR\|O04892); | O04892 (O04892) Cytochrome P450 like_TBP | 184 | 2.00E−47 |
| | cytochrome P450 like_TBP {Nicotiana tabacum} | (EC 1.14.14.1). | | |
| | (GP\|1545805\|dbj\|BAA10929.1\|\|D64052) | | | |
| TC28307 | Putative senescence-associated protein (Fragment) | O04892 (O04892) Cytochrome P450 like_TBP | 59 | 4.00E−16 |
| | (TR\|Q9AVH2); putative senescence-associated protein {Pisum | (EC 1.14.14.1). | | |
| | sativum} | | | |
| TC28316 | T7A14.14 protein (TR\|Q9ZVN4); ESTs gb\|H36249, gb\| | O64410 (O64410) Cytochrome P450 | 49 | 1.00E−12 |
| | AA59732 and gb\|AA651219 come from this gene | monooxygenase (Fragment). | | |
| | {Arabidopsis thaliana} | | | |
| TC28364 | UDP-glucose 4-epimerase GEPI48 (EC 5.1.3.2) | O64410 (O64410) Cytochrome P450 | 70 | 1.00E−13 |
| | (SP\|O65781\|GAE2_CYATE); UDP-GLUCOSE 4-EPIMERASE | monooxygenase (Fragment). | | |
| | GEPI48 (EC 5.1.3.2) (GALACTOWALDENASE) (UDP- | | | |
| | GALACTOSE 4-EPIMERASE) | | | |
| TC28410 | Putative senescence-associated protein (TR\|Q9AVH2) [Sbjct: | O04892 (O04892) Cytochrome P450 like_TBP | 126 | 4.00E−39 |
| | 282, Aligned: 212, Bitscore: 332, Evalue: 3e−090]; putative | (EC 1.14.14.1). | | |
| | senescence-associated protein {Pisum sativum} | | | |
| TC28519 | Cytochrome P450 78A3 (EC 1.14.—.—) | TC28519.1 [546.2078.2043.406] cytochrome | 1040 | 0 |
| | (SP\|O48927\|CP78_SOYBN); cytochrome P450 {Arabidopsis | P450 {Arabidopsis thali . . . | | |
| | thaliana} (GP\|6899886\|emb\|CAB71895.1\|\| | | | |
| | AL138642) | | | |
| TC28637 | Cytochrome P450 (TR\|Q9LUC8); cytochrome P450 | TC28637.1 [258.792.790.17] cytochrome P450 | 455 | e−130 |
| | {Arabidopsis thaliana} | {Arabidopsis thaliana} | | |
| TC28638 | Cytochrome P450 (TR\|Q9LUC8); cytochrome P450 | TC28637.1 [258.792.790.17] cytochrome P450 | 309 | 6.00E−86 |
| | {Arabidopsis thaliana} | {Arabidopsis thaliana} | | |
| TC28751 | Cytochrome P450 (TR\|Q9LUD3); cytochrome P450 | TC28751.1 [314.1142.1140.199] cytochrome | 604 | e−174 |
| | {Arabidopsis thaliana}(GP\|13605897\|gb\|AAK32934.1\| | P450 {Arabidopsis thali . . . | | |
| | AF367347_1\|AF367347) | | | |
| TC28776 | Cytochrome P450 (AT3g14680/MIE1_18) (TR\|Q9LUC6); | TC28776.1 [379.1261.1.1137] cytochrome P450 | 742 | 0 |
| | cytochrome P450 {Arabidopsis thaliana} | {Arabidopsis thalian . . . | | |
| | (GP\|13605897\|gb\|AAK32934.1\|AF367347_1\|AF367347) | | | |
| TC28777 | Cytochrome P450 (TR\|Q9LUD2); putative cytochrome P450 | TC28777.1 [282.848.847.2] putative cytochrome | 575 | e−166 |
| | {Oryza sativa}(GP\|11761114\|dbj\|BAB19104.1\|\|AP002839) | P450 {Oryza sativa . . . | | |
| TC28778 | Cytochrome P450 (TR\|Q9LUD3) | TC28777.1 [282.848.847.2] putative cytochrome | 266 | 4.00E−73 |
| | | P450 {Oryza sativa . . . | | |
| TC29027 | CYTOCHROME P450 (TR\|O65624); cytochrome P450 | TC29027.1 [245.1032.220.954] cytochrome | 496 | e−142 |
| | {Arabidopsis thaliana} | P450 {Arabidopsis thaliana} | | |
| TC29036 | F21F23.15 protein (TR\|Q9LMX7), strong similarity to | TC29036.1 [161.832.830.348] Strong similarity | 311 | 2.00E−86 |
| | cytochrome P-450 from Phalaenopsis | to cytochrome P-45 . . . | | |
| TC29106 | Cytochrome P450 82A1 (EC 1.14.—.—) (CYPLXXXII) | C821_PEA (Q43068) Cytochrome P450 82A1 | | |
| | (SP\|Q43068\|C821_PEA); wound-inducible P450 hydroxylase | (EC 1.14.—.—) (CYPLXXXII) . . . | 431 | 0 |
| | {Pisum sativum} | | | |
| TC29248 | PUTATIVE CYTOCHROME P450 C19A8.04 (TR\|O13820) | O13820 (O13820) PUTATIVE CYTOCHROME | 138 | 5.00E−34 |
| | | P450 C19A8.04 IN CHROMOSOME . . . | | |
| TC29385 | Cytochrom P450-like protein (TR\|Q9LZ31); CYTOCHROME | 67584.m00002#T1E3.20#At5g04660 cytochrom | 381 | 0 |
| | P450 77A3 (EC 1.14.—.—) {Glycine max} | P450 - like protein cyt . . . | | |
| | (GP\|2739010\|gb\|AAB94593.1\|\|AF022464) | | | |
| TC29519 | CYP83D1p (TR\|O48924); CYP83D1p {Glycine max} | TC29519.1 [200.706.626.27] CYP83D1p | 372 | e−105 |
| | (PIR\|T05940\|T05940) | {Glycine max} >PIR\|T05940\|T0 . . . | | |
| TC29852 | Cytochrome P450 98A2 (EC 1.14.—.—) | TC29852.1 [142.630.28.453] CYTOCHROME | 301 | 1.00E−83 |
| | (SP\|O48922\|C982_SOYBN); CYTOCHROME P450 98A2 (EC | P450 98A2 (EC 1.14.—.—). [ . . . | | |
| | 1.14.—.—) {Glycine max} (GP\|2738998\|gb\|AAB94587.1\|\| | | | |
| | AF022458) | | | |
| TC29878 | Cytochrome P450 monooxygenase (TR\|Q9SML3); cytochrome | TC29878.1 [318.954.1.954] cytochrome P450 | 640 | 0 |
| | P450 monooxygenase {Cicer arietinum} | monooxygenase {Cicer a . . . | | |
| TC29957 | Cytochrome P450 71D10 | TC29957.1 [191.574.2.574] CYTOCHROME | 385 | e−109 |
| | (EC 1.14.—.—) (SP\|O48923\|C7DA_SOYBN); | P450 71D10 (EC 1.14.—.—). [ . . . | | |
| | CYTOCHROME P450 71D10 (EC 1.14.—.—) {Glycine max} | | | |
| | (GP\|2739000\|gb\|AAB94588.1\|\|AF022459) | | | |
| TC29997 | Cytochrome P450 (TR\|O04980) | O04980 (O04980) Cytochrome P-450 | 73 | 7.00E−15 |
| | | (Fragment). | | |
| TC30039 | Putative cytochrome P-450 (TR\|Q9C9D1); putative cytochrome | TC30039.1 [205.851.2.616] putative cytochrome | 372 | e−104 |
| | P-450, 4810-6511 {Arabidopsis thaliana}(PIR\|B96769\|B96769) | P-450; 4810-6511 { . . . | | |
| TC30043 | Cytochrome P450 82A4 (EC 1.14.—.—) (P450 CP9) | TC30043.1 [291.1016.1.873] wound-inducible | 541 | e−155 |
| | (SP\|O49859\|C824_SOYBN); wound-inducible P450 | P450 hydroxylase {Pis . . . | | |
| | hydroxylase {Pisum sativum} | | | |
| TC30095 | Cytochrome P450 (TR\|Q9LUD2); cytochrome P450 | TC30095.1 [205.623.8.622] cytochrome P450 | 388 | e−110 |
| | {Arabidopsis thaliana} | {Arabidopsis thaliana} | | |
| TC30145 | Putative cytochrome P450 (TR\|Q9ZUX1); putative cytochrome | TC30145.1 [326.1039.60.1037] putative | 605 | e−175 |
| | P450 {Arabidopsis thaliana} | cytochrome P450 {Arabidops . . . | | |
| | (GP\|13877669\|gb\|AAK43912.1\|AF370593_1 \|AF370593) | | | |
| TC30190 | Ent-kaurenoic acid hydroxylase (TR\|Q9C5Y3); DWARF3 {Zea | TC30190.1 [206.1108.18.635] DWARF3 {Zea | 424 | e−120 |
| | mays} (SP\|Q43246\|C881_MAIZE) | mays} >SP\|Q43246\|C881_MA . . . | | |
| TC30427 | Cytochrome P450 77A3 (EC 1.14.—.—) | TC30427.1 [215.1199.554.1198] Strong | 428 | e−121 |
| | (SP\|O48928\|C773_SOYBN); Strong similarity to gb\|U61231 | similarity to gb\|U61231 cyt . . . | | |
| | cytochrome P450 {Arabidopsis thaliana} | | | |
| TC30574 | Cytochrome P450 86A1 (EC 1.14.—.—) (CYPLXXXVI) | TC30574.1 [325.976.975.1] CYTOCHROME | 654 | 0 |
| | (SP\|P48422\|C861_ARATH); CYTOCHROME P450 86A1 (EC | P450 86A1 (EC 1.14.—.—) (CY . . . | | |
| | 1.14.—.—) (CYPLXXXVI) {Arabidopsis thaliana} | | | |
| TC30622 | Putative ripening-related P-450 enzyme (TR\|Q9M4G8); putative | TC30622.1 [263.792.3.791 ] putative ripening- | 481 | e−138 |
| | ripening-related P-450 enzyme {Vitis vinifera} | related P-450 enzyme . . . | | |
| TC30649 | Ent-kaurenoic acid hydroxylase (TR\|Q9C5Y3); CYTOCHROME | TC30649.1 [193.698.698.120] CYTOCHROME | 409 | e−116 |
| | P450 88A3 (EC 1.14.—.—) {Arabidopsis thaliana} | P450 88A3 (EC 1.14.—.—) . . . | | |
| | (GP\|238858\|gb\|AAB71462) | | | |
| TC30946 | Putative thromboxane-A synthase (TR\|O64853); putative | TC30946.1 [194.634.1.582] putative | 399 | e−113 |
| | thromboxane-A synthase {Arabidopsis thaliana} | thromboxane-A synthase {Arabi . . . | | |
| | (PIR\|T02607\|T02607) | 67284.m00014#F8J2.140#At3g52970 | | |
| TC31146 | Cytochrome P450-like protein (TR\|Q9LF95) | cytochrome P450 - like protein c . . . | 69 | 8.00E−14 |
| TC31263 | Cytochrome P450 93B1 (EC 1.14.—.—) | C9B1_GLYEC (P93149) Cytochrome P450 | 595 | e−172 |
| | (SP\|P93149\|C9B1_GLYEC); CYTOCHROME P450 93B1 (EC | 93B1 (EC 1.14.—.—) ((2S)-fla . . . | | |
| | 1.14.—.—) ((2S)-FLAVANONE 2-HYDROXYLASE) | | | |
| | (LICODIONE SYNTHASE) (FLAVONE SYNT | | | |
| TC31357 | Cytochrome P450 71D9 (EC 1.14.—.—) (P450 CP3) | TC31357.1 [223.671.1.669] CYTOCHROME | 403 | e−114 |
| | (SP\|O81971\|C7D9_SOYBN); CYTOCHROME P450 71D9 (EC | P450 71D9 (EC 1.14.—.—) (P4 . . . | | |
| | 1.14.—.—) (P450 CP3) {Glycine max} | | | |
| | (GP\|3334661\|emb\|CAA71514.1) | | | |
| TC31364 | Cytochrome P450 71A1 (EC 1.14.—.—) (CYPLXXIA1) | TC31364.1 [240.895.721.2] cytochrome p450 | 438 | e−124 |
| | (SP\|P24465\|CP71_PERAE); cytochrome p450 lxxia1 {Persea | lxxia1 {Persea america . . . | | |
| | americana} | | | |
| TC31441 | (S)-N-methylcoclaurine 3′-hydroxylase (TR\|O64901) | 67187.m00109#F10N7.250#At4g31940 | 119 | 8.00E−29 |
| | | Cytochrome P450-like protein cy . . . | | |
| TC31565 | Cytochrome P450 (EC 1.14.14.1) (TR\|Q9XFX1); cytochrome | TC31565.1 [216.649.1.648] cytochrome P450 | 390 | e−110 |
| | P450 {Cicer arietinum} | {Cicer arietinum} | | |
| TC31673 | Cytochrome P450 (TR\|Q9MBE4); cytochrome P450 {Lotus | TC31673.1 [207.668.48.668] cytochrome P450 | 324 | 2.00E−90 |
| | japonicus} | {Lotus japonicus} | | |
| TC31717 | Flavonoid 3′-hydroxylase (TR\|Q9FPN2); flavonoid 3′- | TC40177.2 [224.1612.223.894] CYP83D1p | 109 | 1.00E−25 |
| | hydroxylase {Matthiola incana} | {Glycine max} >PIR\|T05940\| . . . | | |
| TC31893 | Cytochrome P450 (TR\|Q9LUD0); cytochrome P450 | TC31893.1 [269.1834.1782.976] cytochrome | 553 | e−159 |
| | {Arabidopsis thaliana} | P450 {Arabidopsis thali . . . | | |
| TC31895 | Putative membrane related protein (TR\|Q9XIR9); Putative | TC31893.2 [262.1834.1007.222] cytochrome | 178 | 1.00E−45 |
| | membrane related protein {Arabidopsis thaliana} | P450 {Arabidopsis thali . . . | | |
| | (PIR\|D96670\|D96670) | | | |
| TC32040 | Cytochrome P450-like (TR\|Q9LVY7); cytochrome P450-like | TC32040.1 [479.1941.1830.394] cytochrome | 986 | 0 |
| | {Arabidopsis thaliana} | P450-like {Arabidopsis . . . | | |
| TC32102 | Cytochrome P450 72A1 (EC 1.14.14.1) (CYPLXXII) | TC32102.1 [520.2119.1810.251] cytochrome | 1021 | 0 |
| | (SP\|Q05047\|CP72_CATRO); cytochrome p450 lxxii | p450 lxxii hydroxylase) . . . | | |
| | hydroxylase) (ge10h) {Catharanthus roseus} | | | |
| TC32167 | Cytochrome P450 monooxygenaseCYP93D1 (TR\|Q9XHC6); | TC32167.1 [540.1823.1.1620] cytochrome P450 | 1095 | 0 |
| | cytochrome P450 monooxygenaseCYP93D1 {Glycine max} | monooxygenaseCYP93D1 . . . | | |
| TC32250 | Cytochrome P450 (TR\|Q9XGL7); cytochrome P450 {Cicer | TC32250.1 [215.1254.3.647] cytochrome P450 | 440 | e−125 |
| | arietinum} | {Cicer arietinum} | | |
| TC32251 | Monodehydroascorbate reductase (TR\|Q40977); | Q9X4I7 (Q9X4I7) Cytochrome P-450 reductase | 74 | 2.00E−14 |
| | monodehydroascorbate reductase {Pisum sativum} | homolog. | | |
| | (GP\|497120\|gb\|AAA60979.1\|\|U06461) | | | |
| TC32376 | Cytochrome P450 (TR\|Q9LUC5); cytochrome P450 | TC32376.1 [218.703.50.703] cytochrome P450 | 451 | e−129 |
| | {Arabidopsis thaliana} | {Arabidopsis thaliana} | | |
| TC32522 | Cytochrome P450 (TR\|Q9FH76); cytochrome P450 | 67125.m00014#T18B16.200#At4g19230 | 566 | e−178 |
| | {Arabidopsis thaliana} | cytochrome P450 cytochrome P45 . . . | | |
| TC32830 | Cytosolic monodehydroascorbate reductase (TR\|Q9LK94); | Q9X4I7 (Q9X4I7) Cytochrome P-450 reductase | 95 | 5.00E−21 |
| | cytosolic monodehydroascorbate reductase {Arabidopsis | homolog. | | |
| | thaliana} (GP\|14532712\|gb\|AAK64157.1\|\|AY039980) | | | |
| TC32956 | Cytochrome P450 71D10 (EC 1.14.—.—) | TC32956.1 [302.1134.1134.229] | 609 | e−176 |
| | (SP\|O48923\|C7DA_SOYBN); CYTOCHROME P450 | CYTOCHROME P450 71D10 | | |
| | 71D10 (EC 1.14.—.—){Glycine max} (GP\| | (EC 1.14.—.— . . . | | |
| | 2739000\|gb\|AAB94588.1\|\|AF022459) | | | |
| TC33154 | Cytochrome P450 71D11 (EC 1.14.—.—) | TC33154.1 [236.1111.1001.294] putative | 409 | e−116 |
| | (SP\|O22307\|C7DB_LOTJA); putative cytochrome P450 {Lotus | cytochrome P450 {Lotus ja . . . | | |
| | japonicus} | | | |
| TC33255 | Hypothetical 40.1 kDa protein (TR\|Q9LXP4), weak similarity to | Q9X4I7 (Q9X4I7) Cytochrome P-450 reductase | 45 | 5.00E−06 |
| | cytochrome P450 reductase homolog (TR\|Q9X4I7); putative | homolog. | | |
| | protein {Arabidopsis thaliana} (PIR\|T49135\|T49135) | | | |
| TC33268 | Cytochrome P450 81E1 (EC 1.14.—.—) | TC33268.1 [352.1060.3.1058] CYTOCHROME | 607 | e−175 |
| | (SP\|P93147\|C81E_GLYEC); CYTOCHROME | P450 81E1 (EC 1.14.—.—) ( . . . | | |
| | P450 81E1 (EC 1.14.—.—) (ISOFLAVONE 2′- | | | |
| | HYDROXYLASE) (P450 91A4) (CYP GE-3) [Licorice] | | | |
| TC33330 | Putative ripening-related P-450 enzyme (TR\|Q9M4G8); putative | TC33330.1 [379.1141.3.1139] putative ripening- | 687 | 0 |
| | ripening-related P-450 enzyme {Vitis vinifera} | related P-450 enzy . . . | | |
| TC33338 | FLAVONOID 3′,5′-HYDROXYLASE LIKE PROTEIN | O49652 (O49652) CYTOCHROME P450 - | 252 | 1.00E−68 |
| | (TR\|Q9STH8); flavonoid 3′,5′-hydroxylase like protein | LIKE PROTEIN (CYTOCHROME P450- . . . | | |
| | {Arabidopsis thaliana} | | | |
| | (GP\|7267934\|emb\|CAB78276.1\|\|AL161533) | | | |
| TC33416 | Cytochrome P450 71D10 (EC 1.14.—.—) | TC33416.1 [340.1083.63.1082] CYTOCHROME | 632 | 0 |
| | (SP\|O48923\|C7DA_SOYBN); CYTOCHROME P450 71D10 | P450 71D10 (EC 1.14.—.—) . . . | | |
| | (EC 1.14.—.—) {Glycine max} (GP\|2739000\|gb\|AAB94588.1\|\| | | | |
| | AF022459) | | | |
| TC33435 | Cytochrome P450-like protein {Arabidopsis thaliana} | TC32040.1 [479.1941.1830.394] cytochrome | 55 | 7.00E−10 |
| | | P450-like {Arabidopsis . . . | | |
| TC33438 | Cytochrome P450 71D8 (EC 1.14.—.—) (P450 CP7) | TC33438.1 [264.794.1.792] CYTOCHROME | 523 | e−150 |
| | (SP\|O81974\|C7D8_SOYBN); CYTOCHROME P450 71D8 (EC | P450 71D8 (EC 1.14.—.—) (P4 . . . | | |
| | 1.14.—.—) (P450 CP7) {Glycine max} | | | |
| | (GP\|3334667\|emb\|CAA71517.1) | | | |
| TC33577 | Putative NADPH-ferrihemoprotein reductase (TR\|Q9SRU4); | Q9H3M8 (Q9H3M8) NADPH-cytochrome P-450 | 161 | 4.00E−41 |
| | putative NADPH-ferrihemoprotein reductase {Arabidopsis | reductase. | | |
| | thaliana} | | | |
| TC33723 | Cytochrome P450 82A4 (EC 1.14.—.—) (P450 CP9) | TC33723.1 [132.685.2.397] CYTOCHROME | 276 | 5.00E−76 |
| | (SP\|O49859\|C824_SOYBN); CYTOCHROME P450 82A4 (EC | P450 82A4 (EC 1.14.—.—) (P4 . . . | | |
| | 1.14.—.—) (P450 CP9) {Glycine max} | | | |
| | (GP\|2765093\|emb\|CAA71877.1) | | | |
| TC33764 | Cytochrome P450 (TR\|Q9LUD3); cytochrome P450 | TC33764.1 [136.663.256.663] cytochrome P450 | 285 | 9.00E−79 |
| | {Arabidopsis thaliana} | {Arabidopsis thaliana} | | |
| TC33935 | Cytochrome P450 71D9 (EC 1.14.—.—) (P450 CP3) | TC33935.1 [354.1112.3.1064] CYTOCHROME | 706 | 0 |
| | (SP\|O81971\|C7D9_SOYBN); CYTOCHROME P450 7109 (EC | P450 71D9 (EC 1.14.—.—) ( . . . | | |
| | 1.14.—.—) (P450 CP3) {Glycine max} | | | |
| | (GP\|3334661\|emb\|CAA71514.1) | | | |
| TC34093 | Cytochrome P450 monooxygenase (TR\|Q9SML3); cytochrome | Q9SML3 (Q9SML3) Cytochrome P450 | 247 | e−114 |
| | P450 monooxygenase {Cicer arietinum} | monooxygenase (Fragment). | | |
| TC34116 | FLAVONOID 3′,5′-HYDROXYLASE-LIKE PROTEIN | O49650 (O49650) CYTOCHROME P450 LIKE | 74 | 5.00E−15 |
| | (TR\|Q9STI0) | PROTEIN. | | |
| TC34135 | Cytochrome P450 (TR\|Q9AVQ2) | TC34228.1 [197.636.635.45] CYTOCHROME | 244 | 2.00E−73 |
| | | P450 71B2 (EC 1.14.—.—). [ . . . | | |
| TC34228 | Cytochrome P450 71B2 (EC 1.14.—.—) | TC34228.1 [197.636.635.45] CYTOCHROME | 337 | 2.00E−94 |
| | (SP\|O65788\|C722_ARATH); CYTOCHROME P450 71B2 (EC | P450 71B2 (EC 1.14.—.—). [ . . . | | |
| | 1.14.—.—) {Arabidopsis thaliana} | | | |
| TC34688 | Putative ripening-related P-450 enzyme (TR\|Q9M4G8); | TC34688.1 [226.752.42.719] cytochrome P450 | 427 | e−121 |
| | cytochrome P450 monooxygenase {Cicer arietinum} | monooxygenase {Cicer . . . | | |
| TC34694 | Cytochrome P450 monooxygenase (TR\|Q9SML3); cytochrome | TC34694.1 [262.788.1.786] cytochrome P450 | 461 | e−132 |
| | P450 monooxygenase {Cicer arietinum} | monooxygenase {Cicer a . . . | | |
| TC34774 | Putative NADPH-ferrihemoprotein reductase (TR\|Q9SRU4); | Q9HFV3 (Q9HFV3) NADPH cytochrome P450 | 63 | 4.00E−12 |
| | putative NADPH-ferrihemoprotein reductase {Arabidopsis | oxidoreductase isoenzyme 1 . . . | | |
| | thaliana} | | | |
| TC34868 | Putative cytochrome P450 {Arabidopsis thaliana} | 67624.m00019#F18O22.190#At5g14400 | 82 | 1.00E−17 |
| | | putative protein cytochrome P4 . . . | | |
| TC34918 | Cytochrome P450 (TR\|Q9LKH7); cytochrome P450 {Vigna | TC34918.1 [218.658.3.656] cytochrome P450 | 397 | e−112 |
| | radiata} | {Vigna radiata} | | |
| TC35033 | Cytochrome P450 71D9 (EC 1.14.—.—) (P450 CP3) | TC35033.1 [200.675.1.600] CYTOCHROME | 419 | e−119 |
| | (SP\|O81971\|C7D9_SOYBN); CYTOCHROME P450 71D9 (EC | P450 71D9 (EC 1.14.—.—) (P4 . . . | | |
| | 1.14.—.—) (P450 CP3) {Glycine max} | | | |
| | (GP\|3334661\|emb\|CAA71514.1) | | | |
| TC35157 | CYP83D1p (TR\|O48924) | TC29519.1 [200.706.626.27] CYP83D1p | 171 | 1.00E−44 |
| | | {Glycine max} >PIR\|T05940\|T0 . . . | | |
| TC35187 | Putative cytochrome P450 (TR\|O64697); putative cytochrome | TC35187.1 [97.544.544.254] putative | 206 | 3.00E−55 |
| | P450 {Arabidopsis thaliana} (PIR\|T02337\|T02337) | cytochrome P450 {Arabidopsis . . . | | |
| TC35363 | Putative ripening-related P-450 enzyme (TR\|Q9M4G8); putative | TC35363.1 [143.433.431.3] putative ripening- | 253 | 2.00E−69 |
| | ripening-related P-450 enzyme {Vitis vinifera} | related P-450 enzyme . . . | | |
| T035437 | Cytochrom P450-like protein (TR\|Q9SCP8); Cytochrom | TC35437.1 [289.869.2.868]Cytochrom P450- | 511 | e−147 |
| | P450-like protein {Arabidopsis thaliana} (PIR\|T46159\| | like protein {Arabidops . . . | | |
| | T46159) | | | |
| TC35737 | CYP83D1p (TR\|O48924); CYP83D1p {Glycine max} | TC35737.1 [506.1786.43.1560] CYP83D1p | 923 | 0 |
| | (PIR\|T05940\|T05940) | {Glycine max} >PIR\|T05940\| . . . | | |
| TC35738 | Cytochrome P450 83B1 (EC 1.14.—.—) | TC35737.1 [506.1786.43.1560] CYP83D1p | 582 | 0 |
| | (SP\|O65782\|C831_ARATH); CYTOCHROME P450 | {Glycine max) >PIR\|T05940\| . . . | | |
| | 83B1 (EC 1.14.—.—) {Arabidopsis thaliana} (GP\|3164126\|dbj\| | | | |
| | BAA28531) | | | |
| TC35934 | Fatty acid hydroperoxide lyase (TR\|Q9M5J2); fatty acid 9- | CP7B_MOUSE (Q60991) Cytochrome P450 | 64 | 2.00E−11 |
| | hydroperoxide lyase {Cucumis melo} | 7B1 (Oxysterol 7-alpha-hydro . . . | | |
| TC35968 | Cytochrome P450 (TR\|Q9FQL9); cytochrome P450 {Pisum | TC35968.1 [512.1816.47.1582]cytochrome | 1042 | 0 |
| | sativum} | P450 {Pisum sativum} | | |
| TG35969 | Cytochrome P450 (TR\|Q9FQL9); cytochrome P450 {Pisum | TC35969.1 [253.1030.1028.270]cytochrome | 522 | e−150 |
| | sativum} | P450 {Pisum sativum} | | |
| TC36085 | Allene oxide synthase (TR\|Q9M464); allene oxide synthase | 51346.m00093#F3F19.17#At1g13150 putative | 56 | 5.00E−09 |
| | {Lycopersicon esculentum} | cytochrome P450 monooxy . . . | | |
| TC36092 | Cytochrome P450 98A2 | TC36092.1 [265.799.3.797] CYTOCHROME | 538 | e−154 |
| | (EC 1.14.—.—) (SP\|O48922\|C982_SOYBN); CYTOCHROME | P450 98A2 (EC 1.14.—.—). [S . . . | | |
| | P450 98A2 (EC 1.14.—.—) {Glycine max} (GP\|2738998\|gb\| | | | |
| | AAB94587.1\|\|AF022458) | | | |
| TC36216 | Branched-chain amino acid aminotransferase (EC 2.6.1.42) | O04892 (O04892) Cytochrome P450 like_TBP | 45 | 9.00E−08 |
| | (TR\|Q9SNY8); branched-chain amino acid aminotransferase | (EC 1.14.14.1). | | |
| | {Solanum tuberosum} | | | |
| TC36522 | Cytochrome P450 (TR\|Q9XGL7); cytochrome P450 {Cicer | TC36522.1 [200.688.602.3]cytochrome P450 | 287 | 3.00E−79 |
| | arietinum} | {Cicer arietinum} | | |
| TC36707 | F25C20.17 protein (TR\|Q9SAA9); Strong similarity to gb\| | 51344.m00089#F25C20.17#At1g11680 putative | 778 | 0 |
| | U74319 obtusifoliol 14-alpha demethylase (CYP51) {Sorghum | obtusifoliol 14-alpha. . . | | |
| | bicolor} | | | |
| TC36811 | Cytochrome P450 71A1 (EC 1.14.—.—) (CYPLXXIA1) | TC36811.1 [238.1342.628.1341] cytochrome | 489 | e−139 |
| | (SP\|P24465\|CP71_PERAE); cytochrome p450 lxxia1 {Persea | p450 lxxia1 {Persea ame . . . | | |
| | americana} | | | |
| TC36887 | FLAVONOID 3′,5′-HYDROXYLASE LIKE PROTEIN | O49652 (O49652) CYTOCHROME P450 - | 204 | 1.00E−61 |
| | (TR\|Q9STH8); flavonoid 3′,5′-hydroxylase-like protein | LIKE PROTEIN (CYTOCHROME P450- . . . | | |
| | {Arabidopsis thaliana} | | | |
| | (GP\|7267931\|emb\|CAB78273.1\|\| | | | |
| | AL161533) | | | |
| TC36976 | Cytochrome P450 (TR\|Q9LUD2); cytochrome P450 | TC36976.1 [224.797.796.125] cytochrome P450 | 404 | e−114 |
| | {Arabidopsis thaliana} | {Arabidopsis thaliana} | | |
| TC37244 | T12C24.27 (TR\|Q9LN73); T12C24.27 {Arabidopsis thaliana} | 61405.m00078#T12C24.27#At1g12740 | 208 | 1.00E−58 |
| TC37349 | Cytochrome P450 71D10 | cytochrome P450, putative simil . . . | 454 | e−129 |
| | (EC 1.14.—.—) (SP\|O48923\|C7DA_SOYBN); | TC37349.1 [222.835.1.666] CYTOCHROME | | |
| | CYTOCHROME P450 71D10 (EC 1.14.—.—) {Glycine max} | P450 71D10 (EC 1.14.—.—). [ . . . | | |
| | (GP\|2739000\|gb\|AAB94588.1\|\|AF022459) | | | |
| TC37609 | CYP83D1p (Fragment) (TR\|O48924); CYP83D1p | TC37609.1 [202.773.606.1] CYP83D1p {Glycine | 404 | e−114 |
| | {Glycine max} (PIR\|T05940\|T05940) | max} >PIR\|T05940\|T05 . . . | | |
| TC37695 | Cytochrome P450 71D11 (EC 1.14.—.—) | TC37695.1 [201.941.2.604] cytochrome P450 | 386 | e−109 |
| | (SP\|O22307\|C7DB_LOTJA); cytochrome P450 {Nicotiana | {Nicotiana tabacum} | | |
| | tabacum} | | | |
| TC37786 | Cytochrome P450 (TR\|Q9FH76); cytochrome P450 | TC37786.1 [165.1003.1001.507] cytochrome | 345 | 2.00E−96 |
| | {Arabidopsis thaliana} | P450 {Arabidopsis thali . . . | | |
| TC37827 | Cytochrome P450 90A1 (EC 1.14.—.—) | TC37827.1 [168.656.109.612] CYTOCHROME | 302 | 7.00E−84 |
| | (SP\|Q42569\|C901_ARATH); CYTOCHROME P450 90A1 (EC | P450 90A1 (EC 1.14.—.—).. . . | | |
| | 1.14.—.—) {Arabidopsis thaliana} (GP\|853719\|emb\| | | | |
| | CAA60793) | | | |
| TC37938 | Cytochrome P450 71D10 (EC 1.14.—.—) | TC37938.1 [181.736.735.193] CYTOCHROME | 374 | e−105 |
| | (SP\|O48923\|C7DA_SOYBN); CYTOCHROME P450 | P450 71D10 (EC 1.14 —.—) . . . | | |
| | 71D10 (EC 1.14.—.—) {Glycine max} (GP\|2739000\|gb\| | | | |
| | AAB94588.1\|\|AF022459) | | | |
| TC37967 | Cytochrome P450 71D8 (EC 1.14.—.—) (P450 CP7) | TC37967.1 [210.671.41.670] CYTOCHROME | 385 | e−109 |
| | (SP\|O81974\|C7D8_SOYBN); CYTOCHROME P450 71D8 (EC | P450 71D8 (EC 1.14.—.—) (P . . . | | |
| | 1.14.—.—) (P450 CP7) {Glycine max} | | | |
| | (GP\|3334667\|emb\|CAA71517.1) | | | |
| TC37989 | Cytochrome P450 82A3 (EC 1.14.—.—) (P450 CP6) | TC37989.1 [208.669.3.626] CYTOCHROME | 421 | e−119 |
| | (SP\|O49858\|C823_SOYBN); CYTOCHROME P450 82A3 (EC | P450 82A3 (EC 1.14.—.—) (P4 . . . | | |
| | 1.14.—.—) (P450 CP6) {Glycine max} | | | |
| | (GP\|2765091\|emb\|CAA71876.1) | | | |
| TC38094 | Cytochrome P450 71D8 (EC 1.14.—.—) (P450 CP7) | TC38094.1 [243.803.2.730] CYTOCHROME | 429 | e−122 |
| | (SP\|O81974\|C7D8_SOYBN); CYTOCHROME P450 71D8 (EC | P450 71D8 (EC 1.14.—.—) (P4 . . . | | |
| | 1.14.—.—) (P450 CP7) {Glycine max} | | | |
| | (GP\|3334667\|emb\|CAA71517.1) | | | |
| TC38113 | Cytochrome P450 71D10 | TC32956.1 [302.1134.1134.229] | 248 | e−118 |
| | (EC 1.14.—.—) (SP\|O48923\|C7DA_SOYBN); | CYTOCHROME P450 71D10 (EC 1.14.—.— | | |
| | CYTOCHROME P450 71D10 (EC 1.14.—.—) {Glycine max} | . . . | | |
| | (GP\|2739000\|gb\|AAB94588.1\|\|AF022459) | | | |
| TC38419 | Ent-kaurenoic acid oxidase (TR\|Q9AXH9); ent-kaurenoic acid | C881_MAIZE (Q43246) Cytochrome P450 88A1 | 149 | 4.00E−38 |
| | oxidase {Hordeum vulgare} | (EC 1.14.—.—) (DWARF3 p . . . | | |
| TC38630 | Putative cytochrome P450 (TR\|O64631) | TC40352.1 [535.1768.1.1605] putative | 188 | 1.00E−49 |
| | | cytochrome P450 {Arabidopsi . . . | | |
| TC39011 | Putative cytochrome P450 (TR\|O48532); putative cytochrome | TC39011.1 [209.762.1.627] putative cytochrome | 410 | e−116 |
| | P450 {Arabidopsis thaliana} (PIR\|T00934\|T00934) | P450 {Arabidopsis. . . | | |
| TC39332 | Polyubiquitin (TR\|Q38875); chitinase | Q9SML3 (Q9SML3) Cytochrome P450 | 325 | e−108 |
| | | monooxygenase (Fragment). | | |
| TC39429 | Cytochrome P450 monooxygenase (EC 1.14.14.1) (TR\| | TC39429.1 [498.2039.1856.363] cytochrome | 945 | 0 |
| | Q9XFX0); cytochrome P450 {Cicer arietinum} | P450 {Cicer arietinum} | | |
| TC39499 | Putative NADPH-cytochrome P450 reductase (TR\|O04434); | TC39499.1 [734.2582.2580.379] NADPH- | 1313 | 0 |
| | NADPH-cytochrome P450 oxidoreductase (EC 1.—.—.—) — | cytochrome P450 oxidoreducta . . . | | |
| | common tobacco | | | |
| TC39898 | Putative cytochrome P450 (TR\|Q9XIQ1); Putative cytochrome | TC39898.1 [417.1630.1628.378] Putative | 758 | 0 |
| | P450 {Arabidopsis thaliana} | cytochrome P450 {Arabidop . . . | | |
| | (GP\|14334810\|gb\|AAK59583.1\|\|AY035078) | | | |
| TC39899 | Putative cytochrome P450 (TR\|Q9ZUQ6); Putative cytochrome | TC39899.1 [260.818.818.39] Putative | 475 | e−136 |
| | P450 {Arabidopsis thaliana} | cytochrome P450 {Arabidopsis . . . | | |
| | (GP\|14334810\|gb\|AAK59583.1\|\|AY035078) | | | |
| TC39909 | F16N3.6 protein (TR\|Q9SX96), similarity to cytochrome P450 | 51472.m00221#F16N3.40#At1g47630 | 153 | 1.00E−38 |
| | {Arabidopsis thaliana} | cytochrome P450, putative simila . . . | | |
| TC39984 | Putative cytochrome P450 (TR\|Q9C6S0); putative cytochrome | TC39984.1 [453.2055.1.1359] cytochrome | 902 | 0 |
| | P450 {Arabidopsis thaliana} (PIR\|F86441\|F86441) | P450, putative {Arabidops . . . | | |
| TC40170 | CYP71A10 (TR\|O48918); CYP71A10 {Glycine max} | TC40170.1 [265.796.2.796] CYP71A10 {Giycine | 495 | e−142 |
| | (PIR\|T05735\|T05735) | max} >PIR\|T05735\|T05 . . . | | |
| TC40177 | CYP83D1p (TR\|O48924); CYP83D1p {Glycine max} | TC40177.1 [271.1612.800.1612] CYP83D1p | 559 | e−160 |
| | (PIR\|T05940\|T05940) | {Glycine max} >PIR\|T05940 . . . | | |
| TC40226 | Putative cytochrome P450 (TR\|Q9ZUX1); putative cytochrome | TC40226.1 [100.612.393.94] putative | 212 | 7.00E−57 |
| | P450 {Arabidopsis thaliana} | cytochrome P450 {Arabidopsis . . . | | |
| | (GP\|13877669\|gb\|AAK43912.1\|AF370593_1\|AF370593) | | | |
| TC40227 | Putative cytochrome P450 (TR\|Q9ZUX1); putative cytochrome | TC40227.1 [361.1170.2.1084] putative | 650 | 0 |
| | P450 {Arabidopsis thaliana} | cytochrome P450 {Arabidopsi . . . | | |
| | (GP\|13877669\|gb\|AAK43912.1\|AF370593_1\|AF370593) | | | |
| TC40352 | Putative cytochrome P450 (TR\|O64631); putative cytochrome | TC40352.1 [535.1768.1.1605] putative | 992 | 0 |
| | P450 {Arabidopsis thaliana} (PIR\|T00864\|T00864) | cytochrome P450 {Arabidopsi . . . | | |
| TC40404 | Flavone synthase II (TR\|Q9SP27); flavone synthase II | C9B1_GLYEC (P93149) Cytochrome P450 | 167 | 3.00E−43 |
| | {Callistephus chinensis} | 93B1 (EC 1.14.—.—) ((2S)-fla . . . | | |
| TC40527 | Putative cytochrome P450 (TR\|Q9XIQ1); cytochrome P450-like | TC40527.1 [195.721.43.627] cytochrome P450- | 361 | e−101 |
| | protein {Arabidopsis thaliana} | like protein {Arabido . . . | | |
| TC40582 | Monodehydroascorbate reductase (TR\|Q9XEL2); | Q9X4I7 (Q9X4I7) Cytochrome P-450 reductase | 48 | 4.00E−07 |
| | monodehydroascorbate reductase {Brassica juncea} | homolog. | | |
| TC40743 | Putative thromboxane-A synthase (TR\|O64853); putative | O64853 (O64853) Putative thromboxane-A | 271 | e−121 |
| | thromboxane-A synthase {Arabidopsis thaliana} | synthase. | | |
| | (PIR\|T02607\|T02607) | | | |
| TC40811 | Putative cytochrome P450 (TR\|O64631); putative cytochrome | TC40811.1 [512.1608.71.1606] putative | 982 | 0 |
| | P450 {Arabidopsis thaliana} (PIR\|T00404\|T00404) | cytochrome P450 {Arabidops . . . | | |
| TC40856 | Ent-kaurenoic acid oxidase (TR\|Q9AXH9) | C881_MAIZE (Q43246) Cytochrome P450 88A1 | 130 | 4.00E−32 |
| | | (EC 1.14.—.—) (DWARF3 p . . . | | |
| TC40979 | Putative cytochrome P450 (TR\|Q9SJH2); putative cytochrome | TC40979.1 [166.768.38.535] putative | 345 | 1.00E−96 |
| | P450 {Arabidopsis thaliana} (PIR\|A84859\|A84859) | cytochrome P450 {Arabidopsis . . . | | |
| TC41060 | Cytochrome P45071D10 (EC 1.14.—.—) | TC41060.1 [269.1040.809.3] CYTOCHROME | 526 | e−151 |
| | (SP\|O48923\|C7DA_SOYBN); CYTOCHROME P450 | P450 71D9 (EC 1.14.—.—) (P . . . | | |
| | 71D9 (EC 1.14.—.—) (P450 CP3) {Glycine max} | | | |
| | (GP\|3334661\|emb\|CAA71514.1) | | | |
| TC41115 | Putative cytochrome P450 (TR\|O64631); putative cytochrome | TC41115.1 [190.806.2.571] putative cytochrome | 389 | e−110 |
| | P450 {Arabidopsis thaliana} (PIR\|T00404\|T00404) | P450 {Arabidopsis. . . | | |
| TC41116 | Putative cytochrome P450 (TR\|O64631); putative cytochrome | TC41116.1 [208.626.2.625] putative cytochrome | 431 | e−122 |
| | P450 {Arabidopsis thaliana} (PIR\|T00864\|T00864) | P450 {Arabidopsis. . . | | |
| TC41225 | Cytochrome P450 (TR\|Q9FQL9); cytochrome P450 {Pisum | TC35968.1 [512.1816.47.1582] cytochrome | 338 | e−105 |
| | sativum} | P450 {Pisum sativum} | | |
| TC41569 | Cytochrome P450 (EC 1.14.14.1) (TR\|Q9XFX1) | TC33268.1 [352.1060.3.1058]CYTOCHROME | 167 | 5.00E−43 |
| | | P450 81E1 (EC 1.14.—.—) ( . . . | | |
| TC41600 | Cytochrome P450 71A26 (EC 1.14.—.—) | TC41600.1 [269.811.3.809] CYTOCHROME | 480 | e−137 |
| | (SP\|Q9STK7\|C71Q_ARATH); CYTOCHROME P450 71A26 | P450 71A26 (EC 1.14.—.—). [ . . . | | |
| | (EC 1.14.—.—) {Arabidopsis thaliana} | | | |
| | (GP\|4678361\|emb\|CAB4117) | | | |
| TC41677 | Cytochrome P450 (TR\|Q9LUC5); cytochrome P450 | TC41677.1 [236.799.37.744] cytochrome P450 | 480 | e−137 |
| | {Arabidopsis thaliana} | {Arabidopsis thaliana} | | |
| TC41775 | Putative ripening-related P-450 enzyme (TR\|Q9M4G8) | TC34694.1 [262.788.1.786] cytochrome P450 | 155 | 8.00E−40 |
| | | monooxygenase {Cicer a . . . | | |
| TC41781 | Steroid 22-alpha-hydroxylase (DWF4) (TR\|Q9SCQ9); | TC37827.1 [168.656.109.612] CYTOCHROME | 132 | 8.00E−33 |
| | steroid 22-alpha-hydroxylase (DWF4) {Arabidopsis thaliana} | P450 90A1 (EC 1.14.—.—).. . . | | |
| | (PIR\|T46143\|T46143) | | | |
| TC42130 | Flavonoid 3′,5′-hydroxylase 2 (EC | TC35968.1 [512.1816.47.1582] cytochrome | 194 | 3.00E−51 |
| | 1.14.—.—) (SP\|P48419\|C753_PETHY); flavonoid 3′, | P450 {Pisum sativum} | | |
| | 5′-hydroxylase 2 Ixxva3) {Petunia hybrida} | | | |
| TC42218 | Ent-kaurene oxidase (TR\|Q9FQY5); ent-kaurene oxidase | 67950.m00083#T1N24.23#At5g25900 | 300 | 3.00E−83 |
| | {Cucurbita maxima} | cytochrome P450 GA3/67951.m0000 . . . | | |
| TC42253 | Putative ripening-related P-450 enzyme (TR\|Q9M4G8); putative | TC42253.1 [232.1243.1242.547] putative | 422 | e−120 |
| | ripening-related P-450 enzyme {Vitis vinifera} | ripening-related P-450 en . . . | | |
| TC42438 | Putative cytochrome P450 (TR\|O64631); putative cytochrome | TC42438.1 [199.647.42.638] putative | 360 | e−101 |
| | P450 {Arabidopsis thaliana} (PIR\|T00864\|T00864) | cytochrome P450 {Arabidopsis . . . | | |
| TC42500 | Cytochrome P450 71D11 (EC 1.14.—.—) | TC42500.1 [200.600.1.600] putative cytochrome | 405 | e−115 |
| | (SP\|O22307\|C7DB_LOTJA); putative cytochrome P450 {Lotus | P450 {Lotus japoni . . . | | |
| | japonicus} | | | |
| TC42602 | 5-alpha-taxadienol-10-beta-hydroxylase (TR\|Q9AXM6) | TC32040.1 [479.1941.1830.394] cytochrome | 64 | 7.00E−20 |
| | | P450-like {Arabidopsis . . . | | |
| TC42625 | Cytochrome P450 82A3 (EC 1.14.—.—) (P450 CP6) | TC37989.1 [208.669.3.626] CYTOCHROME | 259 | 6.00E−71 |
| | (SP\|O49858\|C823_SOYBN); CYTOCHROME P450 82A3 (EC | P450 82A3 (EC 1.14.—.—) (P4 . . . | | |
| | 1.14.—.—) (P450 CP6) {Glycine max} | | | |
| | (GP\|2765091\|emb\|CAA7l876.1) | | | |
| TC42869 | CYP83D1p (TR\|O48924); CYP83D1p {Glycine max} | TC42869.1 [233.818.818.120] CYP83D1p | 442 | e−126 |
| | (PIR\|T05940\|T05940) | {Glycine max} >PIR\|T05940\|T . . . | | |
| AA660324 | Cytochrome P450 71D11 (EC 1.14.—.—) | AA660324.1 [168.539.3.506] putative | 284 | 1.00E−78 |
| | (SP\|O22307\|C7DB_LOTJA); putative cytochrome P450 {Lotus | cytochrome P450 {Lotus japon . . . | | |
| | japonicus} | | | |
| AI737593 | Cytochrome P450 71D10 (EC 1.14.—.—) | AI737593.1 [133.494.95.493] CYTOCHROME | 271 | 1.00E−74 |
| | (SP\|O48923\|C7DA_SOYBN); CYTOCHROME P450 71D10 | P450 71D10 (EC 1.14.—.—) . . . | | |
| | (EC 1.14.—.—) (GP\|2739000\|gb\|AAB94588.1\|\|AF022459) | | | |
| AJ389053 | Cytochrome P450 (TR\|Q9LUD3) | BE202932.1 [180.638.636.97] cytochrome P450 | 154 | 2.00E−39 |
| | | {Arabidopsis thaliana} | | |
| AL365580 | Cytochrome P450 (TR\|Q9SDM6) | Q9SDM6 (Q9SDM6) Cytochrome P450 | 108 | 8.00E−26 |
| | | (Fragment). | | |
| AL366720 | CYTOCHROME P450 (TR\|O65624) | TC32522.1 [242.1955.1889.1164]cytochrome | 258 | 6.00E−71 |
| | | P450 {Arabidopsis thal . . . | | |
| AL368402 | Cytochrome P450 71D11 (EC 1.14.—.—) | C7DB₋LOTJA (O22307) Cytochrome P450 | 152 | 6.00E−39 |
| | (SP\|O22307\|C7DB₋LOTJA) | 71D11 (EC 1.14.—.—) (Fragment). | | |
| AL368403 | Weak similarity to cytochrome P450 71D11 (EC 1.14.—.—) | C7DB₋LOTJA (O22307) Cytochrome P450 | 44 | 4.00E−06 |
| | (SP\|O22307\|C7DB₋LOTJA) | 71D11 (EC 1.14.—.—) (Fragment). | | |
| AL370043 | Cytochrome P450 76A2 (EC 1.14.—.—) (CYPLXXVIA2) (P | C762₋SOLME (P37122) Cytochrome P450 | 108 | 5.00E−26 |
| | (SP\|P37122\|C762₋SOLME) | 76A2 (EC 1.14.—.—) (CYPLXXVI . . . | | |
| AL372981 | Putative ripening-related P-450 enzyme (TR\|Q9M4G8) | Q9M4G8 (Q9M4G8) Putative ripening-related P- | 110 | 2.00E−26 |
| | | 450 enzyme. | | |
| AL380946 | Ent-kaurene oxidase (TR\|Q9FQY4) | TC30649.1 [193.698.698.120]CYTOCHROME | 191 | 1.00E−50 |
| | | P450 88A3 (EC 1.14.—.—) . . . | | |
| AL381604 | Probable cytochrome P450 311a1 (EC 1.14.—.—) | C311₋DROME (Q9VYQ7) Probable cytochrome | 87 | 4.00E−19 |
| | (SP\|Q9VYQ7\|C311₋DROME) | P450 311a1 (EC 1.14 —.—) . . . | | |
| AL381959 | Cytochrome P450-like protein (TR\|Q9LF95) | 67284.m00014#F8J2.140#At3g52970 | 100 | 3.00E−23 |
| | | cytochrome P450 - like protein c . . . | | |
| AL383331 | Cytochrome P450 (TR\|Q9LUD3) | TC31893.2 [262.1834.1007.222] cytochrome | 153 | 2.00E−39 |
| | | P450 {Arabidopsis thali . . . | | |
| AL384146 | Weak similarity to cytochrome p450 lxxia1 {Persea america} | TC31364.1 [240.895.721.2] cytochrome p450 | 61 | 9.00E−16 |
| | | lxxia1 {Persea america . . . | | |
| AL385275 | Weak similarity to Cytochrome P-450 cyp509A1 (TR\|Q9P493) | Q9P493 (Q9P493) Cytochrome P-450 | 44 | 4.00E−06 |
| | | cyp509A1. | | |
| AL389097 | Cytochrome P450 71D11 (EC 1.14.—.—) | TC35033.1 [200.675.1.600] CYTOCHROME | 119 | 4.00E−29 |
| | (SP\|O22307\|C7DB₋LOTJA) | P450 71D9 (EC 1.14.—.—) (P4 . . . | | |
| AW127462 | Cytochrome P-450-like protein (TR\|Q9FHC0) | Q9FHC0 (Q9FHC0) Cytochrome P-450-like | 172 | 4.00E−45 |
| | | protein. | | |
| AW171770 | CYTOCHROME P450 (TR\|O65624) | TC32522.2 [137.1955.1136.726] cytochrome | 119 | 6.00E−29 |
| | | P450 {Arabidopsis thali . . . | | |
| AW191204 | Cytochrome P450 71D10 (EC 1.14.—.—) | AW191204.1 [143.430.1.429] CYTOCHROME | 258 | 6.00E−71 |
| | (SP\|O48923\|C7DA₋SOYBN); CYTOCHROME P450 71D8 (EC | P450 71D8 (EC 1.14.—.—) (P . . . | | |
| | 1.14.—.—) (P450 CP7) (GP\|3334667\|emb\| | | | |
| | CAA71517.1\|\|Y10493) | | | |
| AW256676 | T12C24.27 (TR\|Q9LN73), similarity to putative cytochrome | 61405.m00078#T12C24.27#At1g12740 | 225 | 8.00E−61 |
| | P450 {Arabidopsis thaliana} | cytochrome P450, putative simil . . . | | |
| AW299043 | Putative cytochrome P450 (TR\|O64631); putative cytochrome | AW299043.1 [197.594.2.592] putative | 404 | e−114 |
| | P450 {Arabidopsis thaliana} (PIR\|T00864\|T00864) | cytochrome P450 {Arabidopsis . . . | | |
| AW299158 | Cytochrome P450-like protein (TR\|Q9LUD1); cytochrome P450- | AW299158.1 [201.686.686.84] cytochrome | 412 | e−117 |
| | like protein {Arabidopsis thaliana} | P450-like protein {Arabid . . . | | |
| AW329314 | Ent-kaurenoic acid oxidase (TR\|Q9AXH9) | C881₋MAIZE (Q43246) Cytochrome P450 88A1 | 114 | 4.00E−27 |
| | | (EC 1.14.—.—) (DWARF3 p . . . | | |
| AW329655 | Cytochrome P450 (TR\|P93148) | P93148 (P93148) Cytochrome P450 | 56 | 6.00E−10 |
| | | (Fragment). | | |
| AW329684 | CYTOCHROME P450 71D12 (EC 1.14.—.—) (TR\|P98183) | TC35033.1 [200.675.1.600] CYTOCHROME | 170 | 6.00E−45 |
| | | P450 71D9 (EC 1.14.—.—) (P4 . . . | | |
| AW559376 | Cytochrome P450 (TR\|Q9FL56); CYTOCHROME P450 93A3 | AW559376.1 [212.636.1.636] CYTOCHROME | 428 | e−122 |
| | (EC 1.14.—.—) (P450 CP5) | P450 93A3 (EC 1.14.—.—) (P . . . | | |
| | (GP\|3334665\|emb\|CAA71516.1\|\| | | | |
| | Y10492) | | | |
| AW574247 | F18O14.38 (TR\|Q9LN32), similarity to putative cytochrome | 60554.m00041#MV111.19#At3g19270 | 98 | 2.00E−22 |
| | P450 {Arabidopsis thaliana} | cytochrome P450, putative simila . . . | | |
| AW586223 | Cytochrome P450 monooxygenase-like protein (TR\|Q9LEX2); | AW586223.1 [89.569.302.568] putative ripening- | 184 | 1.00E−48 |
| | putative ripening-related P-450 enzyme {Vitis vinifera} | related P-450 enzy . . . | | |
| AW684035 | Weak similarity to CYP83D1p {Glycine max} | TC29519.1 [200.706.626.27] CYP83D1p | 67 | 9.00E−14 |
| | | {Glycine max} >PIR\|T05940\|T0 . . . | | |
| AW685151 | Cytochrome P450 93A3 (EC 1.14.—.—) (P450 | AW685151.1 [219.658.1.657] CYTOCHROME | 406 | e−115 |
| | CP5) (SP\|O81973\|C933₋SOYBN); CYTOCHROME P450 93A3 | P450 93A3 (EC 1.14.—.—) (P . . . | | |
| | (EC 1.14.—.—) (P450 CP5) (GP\|3334665\|emb\| | | | |
| | CAA71516.1\|\|Y10492) | | | |
| AW686900 | Cytochrome P450 monooxygenase (TR\|Q9SML3); cytochrome | AW686900.1 [204.615.2.613] cytochrome P450 | 375 | e−106 |
| | P450 monooxygenase {Cicer arietinum} | monooxygenase {Cicer . . . | | |
| AW686916 | Cytochrome P450 71D10 (EC 1.14.—.—) | AW686916.2 [107.395.73.393] CYTOCHROME | 215 | 5.00E−58 |
| | (SP\|O48923\|C7DA₋SOYBN); CYTOCHROME P450 71D10 | P450 71D10 (EC 1.14 —.—) . . . | | |
| | (EC 1.14.—.—) (GP\|2739000\|gb\|AAB94588.1\|\|AF022459) | | | |
| AW687530 | Cytochrome P450 82A1 (EC 1.14.—.—) (CYPLXXXII) | AW687530.1 [82.248.1.246] cytochrome P450 | 165 | 2.00E−43 |
| | (SP\|Q43068\|C821₋PEA); cytochrome P450 monooxygenase | monooxygenase {Pisum s . . . | | |
| | {Pisum sativum} | | | |
| AW687747 | Cytochrome P450 71D10 (EC 1.14.—.—) | TC33416.1 [340.1083.63.1082] CYTOCHROME | 178 | 5.00E−47 |
| | (SP\|O48923\|C7DA₋SOYBN) | P450 71D10 (EC 1.14.—.—) . . . | | |
| AW688786 | Cytochrome P450 71A24 (EC 1.14.—.—) | AW688786.1 [190.611.41.610] cytochrome | 343 | 3.00E−96 |
| | (SP\|Q9STK9\|C71O₋ARATH); cytochrome P450-like protein | P450-like protein (Arabid . . . | | |
| | {Arabidopsis thaliana} (PIR\|T06710\|T06710) | | | |
| AW691814 | Similarity to cytochrome P450 (TR\|Q9LVY3); contains | AW691814.1 [207.663.26.646] contains | 352 | 6.00E−99 |
| | similarity to cytochrome P450 (MAB16.9) {Arabidopsis | similarity to cytochrome P4 . . . | | |
| | thaliana} | | | |
| AW693084 | Cytochrome P450 -like protein {Arabidopsis thaliana} | 67221.m00015#F23K16.140#At4g39510 | 92 | 8.00E−21 |
| | | cytochrome P450-like protein . . . | | |
| AW695208 | Putative cytochrome P450 (TR\|O48532) | C933₋SOYBN (O81973) Cytochrome P450 | 105 | 9.00E−25 |
| | | 93A3 (EC 1.14.—.—) (P450 CP5). | | |
| AW695887 | Ent-kaurene oxidase (TR\|Q9FQY4); putative cytochrome P450 | AW695887.1 [169.621.39.545] putative | 345 | 5.00E−97 |
| | {Arabidopsis thaliana} | cytochrome P450 {Arabidopsi . . . | | |
| AW696374 | CYP71A10 (TR\|O48918); CYP71A10 {Glycine max} | AW696374.1 [218.660.1.654] CYP71A10 | 388 | e−109 |
| | (PIR\|T05735\|T05735) | {Glycine max} >PIR\|T05735\|T0 . . . | | |
| AW697191 | Cytochrome P450 83B1 (EC 1.14.—.—) (SP\|O65782\|C831_{—} | TC29519.1 [200.706.626.27] CYP83D1p | 160 | 2.00E−41 |
| | ARATH) | {Glycine max} >PIR\|T05940\|T0 . . . | | |
| AW774173 | Hypothetical 17.4 kDa protein (Fragment) (TR\|Q9FSZ4) | TC40352.1 [535.1768.1.1605] putative | 150 | 3.00E−38 |
| | | cytochrome P450 {Arabidopsi . . . | | |
| AW774659 | Cytochrome P450 93A3 (EC 1.14.—.—) | C933_SOYBN (081973) Cytochrome P450 | 100 | 4.00E−23 |
| | (SP\|O81973\|C933_S0YBN) | 93A3 (EC 1.14.—.—) (P450 CP5). | | |
| AW774909 | Cytochrome P450 71D11 (EC 1.14.—.—) | AW774909.1 [171.516.3.515] putative | 306 | 3.00E−85 |
| | (SP\|O22307\|C7DB_LOTJA); putative cytochrome P450 {Lotus | cytochrome P450 {Lotus japon . . . | | |
| | japonicus} | | | |
| AW775039 | Putative cytochrome P450 (TR\|Q9ATV0) | 67945.m00012 #F6A4.120#At5g24910 | 67 | 6.00E−13 |
| | | cytochrome P450-like protein fat . . . | | |
| AW775042 | Cytochrome P450 71D11 (EC 1.14.—.—) | AW775042.1 [233.699.1.699] putative | 462 | e−132 |
| | (SP\|O22307\|C7DB_LOTJA); putative cytochrome P450 {Lotus | cytochrome P450 {Lotus japon . . . | | |
| | japonicus} | | | |
| AW980926 | Cytochrome P450 (TR\|Q42700) | TC31893.1 [269.1834.1782.976] cytochrome | 191 | 1.00E−50 |
| | | P450 {Arabidopsis thali . . . | | |
| BE124630 | Cytochrome P450-like protein (TR\|Q9LIC5); cytochrome P450- | BE124630.1 [131.608.187.579] cytochrome | 276 | 4.00E−76 |
| | like protein {Arabidopsis thaliana} | P450-like protein {Arabi . . . | | |
| BE202932 | Cytochrome P450 (AT3g14680/MIE1_18) (TR\|Q9LUC6); | BE202932.1 [180.638.636.97] cytochrome P450 | 362 | e−102 |
| | cytochrome P450 {Arabidopsis thaliana} | {Arabidopsis thaliana} | | |
| BE203749 | Cytochrome P450 71D9 (EC 1.14.—.—) (P450 CP3) | BE203749.2 [134.564.564.163] CYTOCHROME | 290 | 2.00E−80 |
| | (SP\|O81971\|C7D9_SOYBN); CYTOCHROME P450 71D9 (EC | P450 71D9 (EC 1.14.—.—) . . . | | |
| | 1.14.—.—) (P450 CP3) (GP\|3334661\|emb\| | | | |
| | CAA71514.1\|\|Y10490) | | | |
| BE204557 | Cytochrome P450 71B9 (EC 1.14.—.—) | BE204557.1 [124.484.484.113] CYTOCHROME | 267 | 2.00E−73 |
| | (SP\|O64718\|C729_ARATH); CYTOCHROME P450 71B9 (EC | P450 71B9 (EC 1.14 —.—) . . . | | |
| | 1.14.—.—) (GP\|3184281\|gb\| | | | |
| | AAC18928.1\|\|AC004136) | | | |
| BE204704 | Cytochrome P450 (TR\|Q9ZWF2); cytochrome P450 | BE204704.1 [175.526.1.525] cytochrome P450 | 306 | 2.00E−85 |
| | {Glycyrrhiza echinata} | {Glycyrrhiza echinata} | | |
| BE204783 | Cytochrome P450 71D11 (EC 1.14.—.—) | BE204783.1 [192.599.2.577] putative | 402 | e−114 |
| | (SP\|O22307\|C7DB_LOTJA); putative cytochrome P450 {Lotus | cytochrome P450 {Lotus japon . . . | | |
| | japonicus) | | | |
| BE239301 | Cytochrome P450 72A1 (EC 1.14.14.1) (CYPLXXII) | TC31893.2 [262.1834.1007.222] cytochrome | 243 | 3.00E−66 |
| | (SP\|Q05047\|CP72_CATRO) | P450 {Arabidopsis thali . . . | | |
| BE248260 | Flavonoid 3',5'-hydroxylase 1 (EC 1.14.—.—) | TC31364.1 [240.895.721.2] cytochrome p450 | 58 | 5.00E−17 |
| | (SP\|P48418\|C751_PETHY) | lxxia1 {Persea america . . . | | |
| BE248262 | Probable phytosulfokines 3 precursor | 61204.m00055#F13B4.20#At1g13590 | 41 | 1.00E−05 |
| | (SP\|Q9M2Y0\|PSK3_ARATH) | hypothetical protein contains si . . . | | |
| BE248436 | Putative flavonoid 3'-hydroxylase (TR\|Q9FPN5) | Q9M4G8 (Q9M4G8) Putative ripening-related P- | 73 | 3.00E−15 |
| | | 450 enzyme. | | |
| BE315967 | Putative cytochrome P450 (TR\|Q9SJ08) | 51047.m00070#T8F5.12#At1g65340 | 103 | 3.00E−24 |
| | | cytochrome P450, putative similar . . . | | |
| BE316912 | CYP83D1p (Fragment) (TR\|O48924) | TC42869.1 [233.818.818.120] CYP83D1p | 107 | 1.00E−25 |
| | | {Glycine max} >PIR\|T05940\|T . . . | | |
| BE320265 | Cytochrome P450 51 (EC 1.14.14.—) (CYPL1) (P450L1) | CP51_HUMAN (Q16850) Cytochrome P450 51 | 106 | 2.00E−25 |
| | (SP\|Q16850\|CP51_HUMAN) | (EC 1.14.14.—) (CYPLi) (P . . . | | |
| BE322487 | Putative cytochrome P450 {Arabidopsis thaliana} | TC39898.1 [417.1630.1628.378] Putative | 69 | 2.00E−14 |
| | | cytochrome P450 {Arabidop . . . | | |
| BE323562 | F12K21.15 (TR\|Q9LNL3), weak similarity to cytochrome P450- | 67299.m00028#T5P19.280#At3g56630 | 55 | 5.00E−10 |
| | like protein {Arabidopsis thaliana} | cytochrome P450-like protein cy . . . | | |
| BE325451 | Cytochrome P450 97B1 (EC 1.14.—.—) (P450 97A2) | C971_PEA (Q43078) Cytochrome P450 97B1 | 311 | 3.00E−90 |
| | (SP\|Q43078\|C971_PEA) | (EC 1.14.—.—) (P450 97A2). | | |
| BE325883 | Putative cytochrome P450 (TR\|O80823) | C862_ARATH (O23066) Cytochrome P450 | 211 | 1.00E−56 |
| | | 86A2 (EC 1.14.—.—). | | |
| BE940863 | Cytochrome P450 71DB (EC 1.14.—.—) (P450 CP7) | C7D8_SOYBN (O81974) Cytochrome P450 | 287 | 2.00E−79 |
| | (SP\|O81974\|C7DB_SOYBN) | 71D8 (EC 1.14.—.—) (P450 CP7). | | |
| BE941192 | Weak similarity to cytochrome P450 83B1 (EC 1.14.—.—) | TC35738.1 [396.1578.2.1189] CYTOCHROME | 61 | 7.00E−12 |
| | | P450 83B1 (EC 1.14.—.—) . . . | | |
| BE942709 | CYP83D1p (Fragment) (TR\|O48924) | TC35737.1 [506.1786.43.1560] CYP83D1p | 91 | 2.00E−33 |
| | | {Glycine max} >PIR\|T05940\| . . . | | |
| BE943181 | Cytochrome P450 71A23 (EC 1.14.—.—) | C71N_ARATH (Q9STL0) Cytochrome P450 | 159 | 4.00E−41 |
| | (SP\|Q9STL0\|C71N_ARATH) | 71A23 (EC 1.14.—.—). | | |
| BE997641 | Putative cytochrome P450 (TR\|O81077) | 67125.m00014#T18B16.200#At4g19230 | 130 | 1.00E−32 |
| | | cytochrome P450 cytochrome P45 . . . | | |
| BF518570 | Putative ripening-related P-450 enzyme (TR\|Q9M4G8) | TC42253.1 [232.1243.1242.547] putative | 135 | 1.00E−34 |
| | | ripening-related P-450 en . . . | | |
| BF518676 | Hydroperoxide lyase (TR\|Q9STA2) | C306_DROME (Q9VWR5) Probable | 60 | 3.00E−1 1 |
| | | cytochrome P450 306a1 (EC1.14.—.—) . . . | | |
| BF519917 | Cytochrome P450 86A1 (EC 1.14.—.—) (CYPLXXXVI) | C861_ARATH (P48422) Cytochrome P450 | 253 | 3.00E−69 |
| | (SP\|P48422\|C861_ARATH) | 86A1 (EC 1.14.—.—) (CYPLXXXV . . . | | |
| BF521045 | Flavonoid 3'-hydroxylase (EC 1.14.13.21) (TR\|Q9SBQ9) | C981_SORBI (048956) Cytochrome P450 98A1 | 209 | 6.00E−56 |
| | | (EC 1.14.—.—). | | |
| BF631800 | Weak similarity to cytochrome P450 {Arabidopsis thaliana} | TC28637.1 [258.792.790.17] cytochrome P450 | 55 | 1.00E−09 |
| | | {Arabidopsis thaliana} | | |
| BF633591 | Cytochrome P450 71A8 (EC 1.14.—.—) (SP\|Q42716\|C718_{—} | C718_MENPI (Q42716) Cytochrome P450 71A8 | 80 | 1.00E−17 |
| | MENPI) | (EC 1.14.—.—). | | |
| BF641116 | CYP82C1p (TR\|O48925) | 67187.m00109#F10N7.250#At4g31940 | 162 | 1.00E−41 |
| | | Cytochrome P450-like protein cy . . . | | |
| BF641551 | Cytochrome P450 97B2 (EC 1.14.—.—) | C972_SOYBN (O48921) Cytochrome P450 | 357 | e−100 |
| | (SP\|O48921\|C972_SOYBN) | 97B2 (EC 1.14.—.—). | | |
| BF643740 | Cytochrome P450 71A8 (EC 1.14.—.—) (SP\|Q42716\|C718_{—} | C718_MENPI (Q42716) Cytochrome P450 71A8 | 135 | 1.00E−33 |
| | MENPI) | (EC 1.14.—.—). | | |
| BF645909 | PUTATIVE CYTOCHROME P450 (TR\|Q9S833) | 60246.m00084#F1C9.32#At3g01900 putative | 193 | 4.00E−51 |
| | | cytochrome P450 similar . . . | | |
| BF646350 | Cytochrome P450 71A9 (EC 1.14.—.—) (P450 CP1) | TC34228.1 [197.636.635.45] CYTOCHROME | 200 | 2.00E−53 |
| | (SP\|O81970\|C719_SOYBN) | P450 71B2 (EC 1.14.—.—). [ . . . | | |
| BF646830 | Cytochrome P450 82A2 (EC 1.14.—.—) (P450 CP4) | C822_S0YBN (O81972) Cytochrome P450 | 168 | 2.00E−43 |
| | (SP\|O81972\|C822_SOYBN) | 82A2 (EC 1.14.—.—) (P450 CP4). | | |
| BF648194 | Ent-kaurenoic acid hydroxylase (TR\|Q9C5Y2) | TC30190.1 [206.1108.18.635] DWARF3 {Zea | 309 | 6.00E−86 |
| | | mays} >SP\|Q43246\|C881_MA . . . | | |
| BF648401 | Cytochrome P450 (TR\|Q9FRK4) | 60477.m00049#MIE1.11#At3g14610 putative | 155 | 1.00E−39 |
| | | cytochrome P450 similar . . . | | |
| BG448552 | Steroid 22-alpha-hydroxylase (DWF4) (TR\|Q9SCQ9) | 67624.m00019#F18O22.190#At5g14400 | 106 | 6.00E−25 |
| | | putative protein cytochrome P4 . . . | | |
| BG450794 | Cytochrome P450 88A3 (EC 1.14.—.—) (SP\|O23051\|C883_{—} | C883_ARATH (O23051) Cytochrome P450 | 243 | 3.00E−66 |
| | ARATH) | 88A3 (EC 1.14.—.—). | | |
| BG585065 | Weak similarity to cytochrome P-450 aromatase (TR\|Q9DDE7) | Q9DDE7 (Q9DDE7) Cytochrome P-450 | 50 | 8.00E−08 |
| | | aromatase. | | |
| BG585642 | Cytochrome P450 71D11 (EC 1.14.—.—) (Fragment) | TC35033.1 [200.675.1.600] CYTOCHROME | 260 | 4.00E−71 |
| | (SP\|O22307\|C7DB_LOTJA) | P450 71D9 (EC 1.14.—.—) (P4 . . . | | |
| BG586162 | Cytochrome P450 71D11 (EC 1.14.—.—) (Fragment) | AW775042.1 [233.699.1.699] putative | 362 | e−102 |
| | (SP\|O22307\|C7DB_LOTJA) | cytochrome P450 {Lotus japon . . . | | |
| BG587076 | Cytochrome P450 71A26 (EC 1.14.—.—) | TC41600.1 [269.811.3.809] CYTOCHR0ME | 322 | 6.00E−90 |
| | (SP\|Q9STK7\|C71Q_ARATH) | P450 71A26 (EC 1.14.—.—). [ . . . | | |
| BG604173 | Putative cytochrome P450 (TR\|Q9ZUX1) | TC40227.1 [361.1170.2.1084] putative | 69 | 3.00E−14 |
| | | cytochrome P450 {Arabidopsi . . . | | |
| BG645825 | Weak similarity to cytochrome P450 like protein {Arabidopsis | 68071.m00218#AP22.10#At4g36380 | 40 | 1.00E−04 |
| | thaliana} | cytochrome P450 like protein | | |
| BG645829 | Cytochrome P450 71D11 (EC 1.14.—.—) (Fragment) | C7DB_LOTJA (O22307) Cytochrome P450 | 328 | 9.00E−92 |
| | (SP\|O22307\|C7DB_LOTJA) | 71D11 (EC 1.14.—.—) (Fragment). | | |
| BG645906 | Weak similarity to cytochrome P450 83B1 (EC 1.14.—.—) | TC35738.1 [396.1578.2.1189] CYTOCHROME | 45 | 3.00E−13 |
| | | P450 83B1 (EC 1.14.—.—) . . . | | |
| BG646119 | Cytochrome P450-like protein (TR\|Q9SMP5) | 67263.m00002#T8P19.30#At3g48520 | 237 | 3.00E−64 |
| | | cytochrome P450-like protein cyt . . . | | |
| BG647192 | Cytochrome P-450-like protein (TR\|Q9FHC0) | Q9FHC0 (Q9FHC0) Cytochrome P-450-like | 213 | 4.00E−57 |
| | | protein. | | |
| BG647386 | Cytochrome P450 71A26 (EC 1.14.—.—) | TC34228.1 [197.636.635.45] CYTOCHROME | 168 | 1.00E−43 |
| | (SP\|Q9STK7\|C71Q_ARATH) | P450 71B2 (EC 1.14.—.—). [ . . . | | |
| BG648382 | Cytochrome P450-like (TR\|Q9LVY7) | TC32040.1 [479.1941.1830.394] cytochrome | 144 | 3.00E−36 |
| | | P450-like {Arabidopsis . . . | | |
| BG648612 | Cytochrome P450 (TR\|Q9FRK4) | 60477.m00049#MIE1.11#At3g14610 putative | 206 | 9.00E−55 |
| | | cytochrome P450 similar . . . | | |
| BI262798 | Cytochrome P450 71A1 (EC 1.14.—.—) (CYPLXXIA1) | CP71_PERAE (P24465) Cytochrome P450 | 194 | 1.00E−51 |
| | (SP\|P24465\|CP71_PERAE) | 71A1 (EC 1.14.—.—) (CYPLXXIA . . . | | |
| BI267246 | Putative cytochrome P450 {Oryza sativa} | TC28777.1 [282.848.847.2] putative cytochrome | 126 | 3.00E−33 |
| | | P450 {Oryza sativa . . . | | |
| BI268677 | Isoflavone synthase 1 (TR\|Q9M6B9) | TC32250.1 [215.1254.3.647] cytochrome P450 | 80 | 3.00E−17 |
| | | {Cicer arietinum} | | |
| BI270080 | CYTOCHROME P450-LIKE PROTEIN (TR\|Q9SVA8) | 67221.m00014#F23K16.130#At4g39500 | 195 | 9.00E−52 |
| | | cytochrome P450 -like protein . . . | | |
| BI271449 | CYP83D1p (TR\|O48924) | TC35737.1 [506.1786.43.1560] CYP83D1p | 182 | 4.00E−53 |
| | | {Glycine max} >PIR\|T05940\| . . . | | |
| BI272020 | Cytochrome P450 71A1 (EC 1.14.—.—) (CYPLXXIA1) | TC34228.1 [197.636.635.45]CYTOCHROME | 224 | 1.00E−60 |
| | (SP\|P24465\|CP71_PERAE | P450 71B2 (EC 1.14.—.—). [ . . . | | |
| BI272869 | Putative cytochrome P450-related protein (TR\|Q9FRC3) | 67945.m00011#F6A4.110#At5g24900 | 149 | 2.00E−38 |
| | | cytochrome P450-like protein fat . . . | | |
| BI273065 | Cytochrome P450 (TR\|Q9LUD0) | TC28637.1 [258.792.790.17] cytochrome P450 | 128 | 4.00E−46 |
| | | {Arabidopsis thalina} | | |
| BI308384 | CYTOCHROME P450-LIKE PROTEIN (TR\|Q9SVB0) | 67221.m00012#F23K16.110#At4g39480 | 55 | 8.00E−10 |
| | | cytochrome P450-like protein . . . | | |
| BI308532 | CYTOCHROME P450-LIKE PROTEIN (TR\|O49394) | 67187.m00109#F10N7.250#At4g31940 | 172 | 3.00E−53 |
| | | Cytochrome P450-like protein cy . . . | | |
| BI310040 | Cytochrome P450 93A1 (EC 1.14.—.—) | C931_SOYBN (Q42798) Cytochrome P450 | 115 | 7.00E−28 |
| | (SP\|Q42798\|C931_SOYBN) | 93A1 (EC 1.14.—.—). | | |

TABLE 3
Glycosyltransferase ESTs (TCs and singletons) from Medicago truncatula as first round candidates for involvement in
tritepene saponin biosynthesis. Numbers refer to TIGR Medicago Gene Index TC and and singleton numbers.

| TC/EST | Annotation | Best hit in the dataset | Bitscore | Evalue |
|---|---|---|---|---|
| TC28313 | T30F21.10 protein (TR\|Q9SYM5); Similar to dTDP-D-glucose | O05384 (O05384) DNA for | 77 | 6.00E−16 |
| | 4,6-dehydratase {Arabidopsis thaliana} | glycosyltransferase, lytic transglycosyl . . . | | |
| | (GP\|14596091\|gb\|AAK68773.1\|\|AY042833) | | | |
| TC28349 | T30F21.10 protein (TR\|Q9SYM5); Similar to dTDP-D-glucose | O05384 (O05384) DNA for | 74 | 6.00E−15 |
| | 4,6-dehydratase {Arabidopsis thaliana} | glycosyltransferase, lytic transglycosyl . . . | | |
| | (GP\|14596091\|gb\|AAK68773.1 \|\|AY042833) | | | |
| TC28352 | Putative UDP-glycose (Fragment) (TR\|Q9M3H8); putative UDP- | Q9ZWQ5 (Q9ZWQ5) UDP-glycose: flavonoid | 694 | 0 |
| | glycose {Cicer arietinum} | glycosyltransferase. | | |
| TC28388 | T23J18.21 (TR\|Q9LPY1), putative endoxyloglucan glycosyltrase; | 42618.m00036#T9F8.4#At2g06850 putative | 269 | 5.00E−74 |
| | T23J18.21 {Arabidopsis thaliana} (PIR\|G86248\|G86248) | endoxyloglucan glycosyltr . . . | | |
| TC28543 | Cellulase (EC 3.2.1.4) (TR\|Q07524); xyloglucan endo- | 42618.m00036#T9F8.4#At2g06850 putative | 186 | 6.00E−49 |
| | transglycosylase {Carica papaya} | endoxyloglucan glycosyltr . . . | | |
| TC28620 | UDP-D-glucuronate carboxy-lyase (EC 4.1.1.35) (TR\|Q9AV98); | O05384 (O05384) DNA for | 49 | 2.00E−07 |
| | UDP-D-glucuronate carboxy-lyase {Pisum sativum} | glycosyltransferase, lytic transglycosyl . . . | | |
| TC28668 | Putative galactinol synthase (TR\|O22893); Putative galactinol | 60742.m00138#F14C21.47#At1g54940 | 82 | 2.00E−17 |
| | synthase {Arabidopsis thaliana} (PIR\|G96607\|G96607) | hypothetical protein contains s . . . | | |
| TC28828 | UDP-glycose: flavonoid glycosyltransferase (TR\|Q9ZWQ4); UDP- | TC28828.1 [207.865.865.245 Fragment_C] | 409 | e−116 |
| | glycose: flavonoid glycosyltransferase {Vigna mungo} | Tigr: UDP-glycose: flavono . . . | | |
| TC29058 | Putative glucosyl transferase (TR\|Q9ZQ99); putative glucosyl | TC29726.1 [347.1044.4.1044 Fragment_N] | 121 | 1.00E−29 |
| | transferase {Arabidopsis thaliana} (PIR\|C84784\|C84784) | Tigr: UDP-glycose: flavono . . . | | |
| TC29206 | Avr9/Cf-9 rapidly elicited protein 231 precursor (TR\|Q9FQZ3); | 50885.m00110#F20P5.18#At1g70090 | 400 | e−113 |
| | Avr9/Cf-9 rapidly elicited protein 231 {Nicotiana tabacum} | unknown protein similar to putat . . . | | |
| TC29213 | Arbutin synthase (TR\|Q9AR73); arbutin synthase {Rauvolfia | TC37231.1 [261.900.43.825] Tigr: UDP- | 92 | 5.00E−21 |
| | serpentina} | glycose: flavonoid glycosylt . . . | | |
| TC29261 | F4H5.13 protein (TR\|Q9M9Y5), glycosyl transferase 1 | Q9LE59 (Q9LE59) Like glycosyl transferase 1. | 346 | 5.00E−98 |
| | (TR\|Q9LE59); Unknown protein {Arabidopsis thaliana} | | | |
| | (GP\|15028087\|gb\|AAK76574.1\|\|AY045900) | | | |
| TC29480 | Putative UDP-glucose: glycoprotein glucosyltransferase, 10120 | 60125.m0048#T6J4.1#At1g13250 | 49 | 2.00E−07 |
| | (TR\|Q9FVU8); putative UDP-glucose: glycoprotein | hypothetical protein similar to pu . . . | | |
| | glucosyltransferase, 101200-91134 {Arabidopsis thaliana} | | | |
| TC29543 | F18O14.2 (TR\|Q9LN68), similarity to T10M13.14 (PREDICTED | O04253 (O04253) T10M13.14 (PREDICTED | 403 | e−114 |
| | GLYCOSYL TRANSFERASE) (TR\|O04253); F18O14.2 | GLYCOSYL TRANSFERASE). | | |
| | {Arabidopsis thaliana} | | | |
| TC29557 | UDP-glycose: flavonoid glycosyltransferase (TR\|Q9ZWQ5); UDP- | TC29557.1 [231.1071.1070.378 Fragment_C] | 474 | e−135 |
| | glycose: flavonoid glycosyltransferase {Vigna mungo} | Tigr: UDP-glycose: flavo . . . | | |
| TC29558 | UDP-glycose: flavonoid glycosyltransferase (TR\|Q9ZWQ5) | TC29557.1 [231.1071.1070.378 Fragment_C] | 139 | 2.00E−35 |
| | | Tigr: UDP-glycose: flavo . . . | | |
| TC29660 | Anthocyanidin-3-glucoside rhamnosyltransferase-like | TC29726.1 [347.1044.4.1044 Fragment_N] | 57 | 2.00E−10 |
| | (TR\|Q9LTA3); anthocyanidin-3-glucoside rhamnosyltransferase- | Tigr: UDP-glycose: flavono . . . | | |
| | like {Arabidopsis thaliana} | | | |
| TC29719 | Putative ribophorin I (dolichyl-diphosphooligosaccharide-pro | TC29719.1 [230.949.947.258 Fragment_C] | 430 | e−122 |
| | (TR\|Q9SFX3); putative ribophorin I (dolichyl- | Tigr: putative ribophorin . . . | | |
| | diphosphooligosaccharide-protein glycosyltransferase), | | | |
| | 43789-46748 {Arabidopsis thaliana} | | | |
| TC29726 | UDP-glycose: flavonoid glycosyltransferase (TR\|Q9ZWQ5); UDP- | TC29726.1 [347.1044.4.1044 Fragment_N] | 611 | e−177 |
| | glycose: flavonoid glycosyltransferase {Vigna mungo} | Tigr: UDP-glycose: flavono . . . | | |
| TC29915 | Similarity to unknown protein (TR\|Q9FH36), similarity to glycosyl | Q9LE59 (Q9LE59) Like glycosyl transferase 1. | 87 | 6.00E−27 |
| | transferase 1 (TR\|Q9LE59); contains similarity to unknown | | | |
| | protein, K5F14.3 {Arabidopsis thaliana} (gb\|AAF26170.1) | | | |
| TC30007 | Glucosyltransferase-like protein (TR\|Q9LXV0); | TC29726.1 [347.1044.4.1044 Fragment_N] | 73 | 6.00E−22 |
| | glucosyltransferase-like protein {Arabidopsis thaliana} | Tigr: UDP-glycose: flavono . . . | | |
| | (PIR\|T49903\|T49903) | | | |
| TC30011 | Putative glucosyltransferase (TR\|O64732); putative | Q9LTH2 (Q9LTH2) UDP-glycose: flavonoid | 43 | 5.00E−06 |
| | glucosyltransferase {Arabidopsis thaliana} (PIR\|T00583\|T00583) | glycosyltransferase-like. | | |
| TC30139 | F6F9.24 protein (TR\|Q9FXG9); Unknown Protein {Arabidopsis | 51942.m00184#F14P1 .19#At1g19710 | 174 | 8.00E−46 |
| | thaliana} (PIR\|A86330\|A86330) | hypothetical protein contains Pf . . . | | |
| TC30265 | Glycosyl transferases-like protein (TR\|Q9LSB5); glycosyl | TC30265.1 [93.624.1.279 Fragment_C] Tigr: | 194 | 8.00E−52 |
| | transferases-like protein {Arabidopsis thaliana} | glycosyl transferases- . . . | | |
| TC30461 | Betanidin-5-O-glucosyltransferase (TR\|Q9SMG6); | TC29726.1 [347.1044.4.1044 Fragment_N] | 165 | 3.00E−48 |
| | glucosyltransferase-like protein {Arabidopsis thaliana} | Tigr: UDP-glycose: flavono . . . | | |
| | (PIR\|T46162\|T46162) | | | |
| TC30542 | Hypothetical 60.3 kDa protein (TR\|Q9LXS3), glycosyl transferase | Q9LE59 (Q9LE59) Like glycosyl transferase 1. | 150 | 2.00E−38 |
| | 1 (TR\|Q9LE59); putative protein {Arabidopsis thaliana} | | | |
| | (PIR\|T49162\|T49162) | | | |
| TC30549 | Xyloglucan endotransglycosylase XET2 (EC 2.4.1.207) | 42618.m00036#T9F8.4#At2g06850 putative | 118 | 9.00E−29 |
| | (TR\|Q9LLC2); xyloglucan endotransglycosylase XET2 | endoxyloglucan glycosyltr . . . | | |
| | {Asparagus officinalis} | | | |
| TC30813 | HYPOTHETICAL 19.6 kDa PROTEIN (TR\|O23514), weak | O05696 (O05696) Glycosyl transferase. | 51 | 2.00E−08 |
| | similarity to glycosyl transferase (TR\|O05696); hypothetical | | | |
| | protein {Arabidopsis thaliana} | | | |
| | (GP\|2245026\|emb\|CAB10446.1\|\|Z97341) | | | |
| TC30847 | At2g38650 protein (TR\|Q9ZVI7), glycosyl transferase 1 | Q9LE59 (Q9LE59) Like glycosyl transferase 1. | 139 | 3.00E−35 |
| | (TR\|Q9LE59); hypothetical protein {Arabidopsis thaliana} | | | |
| | (PIR\|F84807\|F84807) | | | |
| TC31133 | UDP-glucose: flavonoid 7-O-glucosyltransferase (TR\|Q9SXF2) | TC29557.1 [231.1071.1070.378 Fragment_C] | 60 | 1.00E−11 |
| | | Tigr: UDP-glycose: flavo . . . | | |
| TC31142 | Putative glucosyltransferase (TR\|O64732); putative | Q9LTH2 (Q9LTH2) UDP-glycose: flavonoid | 133 | 4.00E−33 |
| | glucosyltransferase {Arabidopsis thaliana} (PIR\|T00583\|T00583) | glycosyltransferase-like. | | |
| TC31145 | UDP glucose: flavonoid 3-o-glucosyltransferase-like protein | 60533.m00038#MDC8.15#At3g16520 | 82 | 8.00E−8 |
| | (TR\|Q9LFJ8); UDP-galactose: flavonol 3-O-galactosyltransferase | putative glucosyltransferase simi . . . | | |
| | {Petunia x hybrida} | | | |
| TC31211 | F21O3.4 protein (TR\|Q9SRT3); putative glucosyltransferase | Q97J01 (Q97J01) Glycosyltransferase, | 41 | 5.00E−06 |
| | {Cicer arietinum} | involved in cell wall bioge . . . | | |
| TC31232 | UDP-GLUCOSE GLUCOSYLTRANSFERASE (T16E15.3 | Q9LTH2 (Q9LTH2) UDP-glycose: flavonoid | 38 | 6.00E−08 |
| | PROTEIN) (TR\|Q9ZWJ3): UDP-glucose glucosyltransferase | glycosyltransferase-like. | | |
| | {Arabidopsis thaliana} | | | |
| | (GP\|9392679\|gb\|AAF87256.1\|AC068562_3\|AC068562) | | | |
| TC31370 | Arbutin synthase (TR\|Q9AR73); arbutin synthase {Rauvolfia | TC37231.1 [261 .900.43.825] Tigr: UDP- | 42 | 2.00E−09 |
| | serpentina} | glycose: flavonoid glycosylt . . . | | |
| TC31459 | Arbutin synthase (TR\|Q9AR73) | 60533.m00038#MDC8.15#At3g16520 | 49 | 1.00E−12 |
| | | putative glucosyltransferase simi . . . | | |
| TC31621 | Brassinosteroid-regulated protein BRU1 | 42618.m00036#T9F8.4#At2g06850 putative | 213 | 2.00E−57 |
| | (SP\|P35694\|BRU1_SOYBN); brassinosteroid-regulated protein | endoxyloglucan glycosyltr . . . | | |
| | bru 1 {Glycine max} | | | |
| TC31672 | UDP-GLUCOSE GLUCOSYLTRANSFERASE (T16E15.3 | Q9LTH2 (Q9LTH2) UDP-glycose: flavonoid | 79 | 9.00E−17 |
| | PROTEIN) (TR\|Q9ZWJ3); UDP-glucose glucosyltransferase | glycosyltransferase-like. | | |
| | {Arabidopsis thaliana} | | | |
| | (GP\|9392679\|gb\|AAF87256.1\|AC068562_3_AC068562) | | | |
| TC32246 | PUTATIVE XYLOGLUCAN ENDOTRANSGLYCOSYLASE | 42618.m00036#T9F8.4#At2g06850 putative | 287 | 1.00E−79 |
| | (TR\|Q9ZR10); putative xyloglucan endotransglycosylase | endoxyloglucan glycosltr . . . | | |
| | {Arabidopsis thaliana} (GP\|4262149\|gb\|AAD14449.1\|\|AC005275) | | | |
| TC32310 | T16E15.2 protein (TR\|Q9LMF0); Strong similarity to UDP-glucose | Q9LTH2 (Q9LTH2) UDP-glycose: flavonoid | 82 | 2.00E−17 |
| | glucosyltransferase {Arabidopsis thaliana} (gb\|AB016819) | glycosyltransferase-like. | | |
| TC32311 | UDP-GLUCOSE GLUCOSYLTRANSFERASE (T16E15.3 | Q9LTH2 (Q9LTH2) UDP-glycose: flavonoid | 195 | 8.00E−52 |
| | PROTEIN) (PUTATIVE (TR\|Q9ZWJ3); UDP-glucose | glycosyltransferase-like. | | |
| | glucosyltransferase {Arabidopsis thaliana} | | | |
| | (GP\|9392679\|gb\|AAF87256.1\|AC068562_3\|AC068562) | | | |
| TC32312 | UDP-GLUCOSE GLUCOSYLTRANSFERASE (T16E15.3 | Q9LTH2 (Q9LTH2) UDP-glycose: flavonoid | 74 | 2.00E−15 |
| | PROTEIN) (TR\|Q9ZWJ3); UDP-glucose glucosyltransferase | glycosyltransferase-like. | | |
| | {Arabidopsis thaliana} | | | |
| | (GP\|9392679\|gb\|AAF87256.1\|AC068562_3\|AC068562) | | | |
| TC32329 | DTDP-glucose 4-6-dehydratase (TR\|Q9SMJ5); dTDP-glucose 4- | O05384 (O05384) DNA for | 52 | 2.00E−08 |
| | 6-dehydratase {Cicer arietinum} (PIR\|T51252\|T51252) | glycosyltransferase, lytic transglycosyl . . . | | |
| TC32362 | F20P5.18 protein (TR\|O04536); ESTs | 50885.m00110#F20P5.18#At1g70090 | 438 | e−125 |
| | gb\|N38288, gb\|T43486, gb\|AA395242 come from this gene. | unknown protein similar to putat . . . | | |
| | {Arabidopsis thaliana} | | | |
| TC32409 | Xyloglucan endotransglycosylase-related protein (TR\|Q38908); | 42618.m00036#T9F8.4#At2g06850 putative | 194 | 2.00E−51 |
| | xyloglucan endotransglycosylase-related protein {Arabidopsis | endoxyloglucan glycosyltr . . . | | |
| | thaliana} (PIR\|S71223\|S71223) | | | |
| TC32503 | Betanidin-5-O-glucosyltransferase (TR\|Q9SMG6); putative | Q9ZWQ5 (Q9ZWQ5) UDP-glycose: flavonoid | 331 | 2.00E−92 |
| | glucosyltransferase {Arabidopsis thaliana} (PIR\|E84529\|E84529) | glycosyltransferase. | | |
| TC32536 | UDP-GLUCOSE GLUCOSYLTRANSFERASE (T16E15.3 | Q9LTH2 (Q9LTH2) UDP-glycose: flavonoid | 112 | 1.00E−26 |
| | PROTEIN) (PUTATIVE (TR\|Q9ZWJ3); UDP-glucose | glycosyltransferase-like. | | |
| | glucosyltransferase {Arabidopsis thaliana} | | | |
| | (GP\|9392679\|gb\|AAF87256.1\|AC068562_3\|AC068562) | | | |
| TC32537 | UDP-GLUCOSE GLUCOSYLTRANSFERASE (T16E15.3 | Q9LTH2 (Q9LTH2) UDP-glycose: flavonoid | 52 | 4.00E−09 |
| | PROTEIN) (TR\|Q9ZWJ3); Putative UDP-glucose | glycosyltransferase-like. | | |
| | glucosyltransferase {Arabidopsis thaliana} (PIR\|H86356\|H86356) | | | |
| TC32571 | Putative anthocyanidin-3-glucoside rhamnosyltransferase | Q9LTH2 (Q9LTH2) UDP-glycose: flavonoid | 127 | 5.00E−31 |
| | (TR\|Q9ZQ54); anthocyanidin-3-glucoside rhamnosyltransferase- | glycosyltransferase-like. | | |
| | like {Arabidopsis thaliana} | | | |
| TC32579 | Cellulose synthase isolog (TR\|O22989); cellulose synthase | Q971S9 (Q97IS9) Glycosyltransferases, | 43 | 1.00E−05 |
| | catalytic subunit-like protein {Arabidopsis thaliana} | involved in cell wall biog . . . | | |
| | (GP\|7269248\|emb\|CAB81317.1) | | | |
| TC32669 | UDP-glycose: flavonoid glycosyltransferase (TR\|Q9ZWQ5); | TC29726.1 [347.1044.4.1044 Fragment_N] | 493 | e−141 |
| | putative glucosyltransferase {Arabidopsis thaliana} | Tigr: UDP-glycose: flavono . . . | | |
| TC32716 | NUCLEOTIDE SUGAR EPIMERASE-LIKE PROTEIN | O05384 (O05384) DNA for | 41 | 5.00E−05 |
| | (TR\|Q9STI6); nucleotide sugar epimerase-like protein | glycosyltransferase, lytic transglycosyl . . . | | |
| | {Arabidopsis thaliana} | | | |
| | (GP\|7267926\|emb\|CAB78268.1\|\|AL161533) | | | |
| TC32906 | Arbutin synthase (TR\|Q9AR73) | 60533.m00038#MDC8.15#At3g16520 | 87 | 3.00E−24 |
| | | putative glucosyltransferase simi . . . | | |
| TC33031 | Weak similarity to glycosyl transferases-like protein (TR\|Q9LSB5) | Q9LSB5 (Q9LSB5) Glycosyl transferases-like | 45 | 1.00E−06 |
| | | protein. | | |
| TC33217 | UDP-glucose: salicylic acid glucosyltransferase (TR\|Q9M6E7) | Q9LTH2 (Q9LTH2) UDP-glycose: flavonoid | 54 | 4.00E−09 |
| | | glycosyltransferase-like. | | |
| TC33241 | UDP-glucose: salicylic acid glucosyltransferase (TR\|Q9M6E7); | 60533.m00038#MDC8.15#At3g16520 | 162 | 9.00E−42 |
| | putative glucosyltransferase {Arabidopsis thaliana} | putative glucosyltransferase simi . . . | | |
| | (PIR\|T00507\|T00507) | | | |
| TC33275 | Similarity to glycosyl transferase 1 (TR\|Q9LE59), 68 kDa protein | Q9LE59 (Q9LE59) Like glycosyl transferase 1. | 114 | 2.00E−30 |
| | (TR\|Q9M3Y6); 68 kDa protein {Cicer arietinum} | | | |
| TC33320 | HYPOTHETICAL 32.3 kDa PROTEIN (TR\|Q9SZB1); | 60742.m00138#F14C21.47#At1g54940 | 67 | 3.00E−13 |
| | hypothetical protein {Arabidopsis thaliana} | hypothetical protein contains s . . . | | |
| | (GP\|7270282\|emb\|CAB80051.1\|\|AL161583) | | | |
| TC33364 | Putative dolichyl-phosphate beta-glucosyltransferase | Q9CH63 (Q9CH63) Glycosyl transferase. | 66 | 5.00E−13 |
| | (TR\|Q9SLNO); putative dolichyl-phosphate beta- | | | |
| | glucosyltransferase {Arabidopsis thaliana} (PIR\|T00571\|T00571) | | | |
| TC33566 | Flavonol 3-O-glucosyltransferase-like protein (TR\|Q9FN26); | MGT_STRLI (Q54387) Macrolide | 43 | 6.00E−06 |
| | Similar to Flavonol 3-O-Glucosyltransferase {Arabidopsis | glycosyltransferase (EC 2.4.1.—). | | |
| | thaliana} (PIR\|F96672\|F96672) | | | |
| TC33614 | AT3g21750/MSD21_6 (TR\|Q9ASY6), similarity to putative | 60533.m00038#MDC8.15#At3g16520 | 65 | 8.00E−13 |
| | glucosyltransferase {Arabidopsis thaliana} | putative glucosyltransferase simi . . . | | |
| TC33618 | Glycosyl transferases-like protein (TR\|Q9LSB5); glycosyl | TC33618.1 [308.926.2.925 Fragment_I] Tigr: | 593 | e−172 |
| | transferases-like protein {Arabidopsis thaliana} | glycosyl transferases . . . | | |
| TC33687 | Gb\|AAC34345.1 (TR\|Q9LSB1); Unknown protein {Arabidopsis | 60742.m00138#F14C21.47#At1g54940 | 188 | 2.00E−49 |
| | thaliana} (PIR\|T00444\|T00444) | hypothetical protein contains s . . . | | |
| TC33732 | UTP-glucose glucosyltransferase (TR\|Q9LSY8); | Q9ZWQ4 (Q9ZWQ4) UDP-glycose: flavonoid | 87 | 1.00E−19 |
| | glucosyltransferase {Nicotiana tabacum} | glycosyltransferase (Fragm . . . | | |
| TC33759 | Putative glucosyl transferase (TR\|Q9ZQ98); glucosyltransferase- | TC29726.1 [347.1044.4.1044 Fragment_N] | 102 | 4.00E−24 |
| | like protein {Arabidopsis thaliana} (PIR\|T46162\|T46162) | Tigr: UDP-glycose: flavono . . . | | |
| TC33772 | Flavonol 3-O-glucosyltransferase-like (TR\|Q9LVW3); putative | Q9ZWQ5 (Q9ZWQ5) UDP-glycose: flavonoid | 63 | 4.00E−12 |
| | flavonol 3-O-glucosyltransferase {Arabidopsis thaliana} | glycosyltransferase. | | |
| | (PIR\|F84618\|F84618) | | | |
| TC33774 | F9D12.19 protein (TR\|O81504), weak similarity to cyclodextrin | O30565 (O30565) Cyclodextrin | 49 | 6.00E−08 |
| | glycosyltransferase (EC 2.4.1.19) (TR\|O30565) | glycosyltransferase (EC 2.4.1.19). | | |
| TC33811 | Hypothetical 43.6 kDa protein (TR\|Q9LFB0); putative protein | O30565 (O30565) Cyclodextrin | 45 | 6.00E−07 |
| | {Arabidopsis thaliana} (PIR\|T45966\|T45966) | glycosyltransferase (EC 2.4.1.19). | | |
| TC33925 | UDP-GLUCOSE GLUCOSYLTRANSFERASE (T16E15.3 | Q9LTH2 (Q9LTH2) UDP-glycose: flavonoid | 219 | 6.00E−59 |
| | PROTEIN) (PUTATIVE (TR\|Q9ZWJ3); probable UDP- | glycosyltransferase-like. | | |
| | glucuronosyltransferase (EC 2.4.1.—) - garden pea | | | |
| TC34114 | UDP-glucose: salicylic acid glucosyltransferase (TR\|Q9M6E7); | Q9ZWQ4 (Q9ZWQ4) UDP-glycose: fiavonoid | 143 | 3.00E−36 |
| | Similar to indole-3-acetate beta-glucosyltransferase {Arabidopsis | glycosyltransferase (Fragm . . . | | |
| | thaliana} (PIR\|A86191\|A86191) | | | |
| TC34190 | UDP-glycose: flavonoid glycosyltransferase (Fragment) | Q9ZWQ3 (Q9ZWQ3) UDP-glycose: flavonoid | 115 | 4.00E−28 |
| | (TR\|Q9ZWQ3) | glycosyltransferase (Fragm . . . | | |
| TC34796 | UDP-glycose: flavonoid glycosyltransferase (TR\|Q9ZWQ5) | TC36484.1 [271.1081.1080.268 Fragment_C] | 87 | 9.00E−20 |
| | | Tigr: UDP-glycose: flavo . . . | | |
| TC34907 | Arbutin synthase (TR\|Q9AR73); arbutin synthase {Rauvolfia | 60533.m00038#MDC8.15#At3g16520 | 165 | 1.00E−42 |
| | serpentina} | putative glucosyltransferase simi . . . | | |
| TC35060 | UDP-glycose: flavonoid glycosyltransferase (TR\|Q9ZWQ5) | TC36484.1 [271.1081.1080.268 Fragment_C] | 175 | 3.00E−46 |
| | | Tigr: UDP-glycose: flavo . . . | | |
| TC35085 | Arbutin synthase (TR\|Q9AR73) | 60533.m00038#MDC8.15#At3g16520 | 79 | 6.00E−17 |
| | | putative glucosyltransferase simi . . . | | |
| TC35664 | Putative glucosyltransferase (TR\|O80505); putative | TC35664.1 [207.939.623.3 Fragment_N]Tigr: | 395 | e−112 |
| | glucosyltransferase {Arabidopsis thaliana} (PIR\|T01593\|T01593) | putative glucosyltran . . . | | |
| TC35768 | Putative anthocyanidin-3-glucoside rhamnosyltransferase | Q9ZWQ5 (Q9ZWQ5) UDP-glycose: flavonoid | 144 | 2.00E−36 |
| | (TR\|Q9ZQ54); anthocyanidin-3-glucoside rhamnosyltransferase | glycosyltransferase. | | |
| | {Arabidopsis thaliana} | | | |
| TC35769 | Anthocyanidin-3-glucoside rhamnosyltransferase (TR\|Q9LSM0) | Q9LTH2 (Q9LTH2) UDP-glycose: flavonoid | 40 | 2.00E−05 |
| | | glycosyltransferase-like. | | |
| TC35770 | Putative anthocyanidin-3-glucoside rhamnosyltransferase | TC29557.1 [231.1071.1070.378 Fragment_C] | 42 | 2.00E−10 |
| | (TR\|Q9ZQ54); anthocyanidin-3-glucoside rhamnosyltransferase- | Tigr: UDP-glycose: flavo . . . | | |
| | like {Arabidopsis thaliana} | | | |
| TC35771 | Putative anthocyanidin-3-glucoside rhamnosyltransferase | Q9ZWQ5 (Q9ZWQ5) UDP-glycose: flavonoid | 60 | 3.00E−11 |
| | (TR\|Q9ZQ54); anthocyanidin-3-glucoside rhamnosyltransferase- | glycosyltransferase. | | |
| | like {Arabidopsis thaliana} | | | |
| TC35772 | Endo-xyloglucan transferase precursor (TR\|Q41638); endo- | 42618.m00036#T9F8.4#At2g06850 putative | 469 | e−134 |
| | xyloglucan transferase {Vigna angularis} (PIR\|A49539\|A49539) | endoxyloglucan glycosyltr . . . | | |
| TC35773 | Xyloglucan endotransglycosylase 1 (TR\|Q9ZRV1); xyloglucan | 42618.m00036#T9F8.4#At2g06850 putative | 282 | 6.00E−78 |
| | endotransglycosylase XET2 {Asparagus officinalis} | endoxyloglucan glycosyltr . . . | | |
| TC35774 | Brassinosteroid-regulated protein BRU1 | 42618.m00036#T9F8.4#At2g06850 putative | 292 | 4.00E−81 |
| | (SP\|P35694\|BRU1_SOYBN); brassinosteroid-regulated protein | endoxyloglucan glycosyltr . . . | | |
| | bru1 {Glycine max} | | | |
| TC35775 | Brassinosteroid-regulated protein BRU1 | 42618.m00036#T9F8.4#At2g06850 putative | 292 | 6.00E−81 |
| | (SP\|P35694\|BRU1_SOYBN); brassinosteroid-regulated protein | endoxyloglucan glycosyltr . . . | | |
| | bru1 {Glycine max} | | | |
| TC35838 | Nucleotide sugar epimerase-like protein (TR\|Q9MOB6); | O05384 (O05384) DNA for | 46 | 1.00E−06 |
| | nucleotide sugar epimerase-like protein {Arabidopsis thaliana} | glycosyltransferase, lytic transglycosyl . . . | | |
| | (PIR\|A85356\|A85356) | | | |
| TC35915 | UDP-glucose: salicylic acid glucosyltransferase (TR\|Q9M6E7); | Q9LTH2 (Q9LTH2) UDP-glycose: flavonoid | 65 | 2.00E−19 |
| | 80099 {Arabidopsis thaliana} (PIR\|H86190\|H86190) | glycosyltransferase-like. | | |
| TC36122 | INDOLE−3-AC ETATE BETA-GLUCOSYLTRANSFERASE | Q9LTH2 (Q9LTH2) UDP-glycose: flavonoid | 43 | 4.00E−06 |
| | (TR\|O23400); indole-3-acetate beta-glucosyltransferase like | glycosyltransferase-like. | | |
| | protein {Arabidopsis thaliana} (GP\|2244905\|emb\|CAB1032) | | | |
| TC36123 | Limonoid UDP-glucosyltransferase (EC 2.4.1.210) | Q9LTH2 (Q9LTH2) UDP-glycose: flavonoid | 140 | 3.00E−35 |
| | (SP\|Q9MB73\|LGT_CITUN); LIMONOID UDP- | glycosyltransferase-like. | | |
| | GLUCOSYLTRANSFERASE (EC 2.4.1.210) (LIMONOID | | | |
| | GLUCOSYLTRANSFERASE) (LIMONOID GTASE) | | | |
| TC36130 | Putative ribophorin \|(TR\|Q9ZUA0); putative ribophorin \| | 60052.m00002#F15M4.10#At1g76400 | 120 | 2.00E−29 |
| | {Arabidopsis thaliana} (PIR\|C84428\|C84428) | putative ribophorin \|(dolichyl- . . . | | |
| TC36131 | Putative ribophorin \| (TR\| Q9ZUA0); putative ribophorin \| | 60052.m00002·0F15M4.10.0 At1g76400 | 266 | 9.00E−87 |
| | {Arabidopsis thaliana} (PIR\|C84428\|C84428) | putative ribophorin \|(dolichyl- . . . | | |
| TC36146 | Hypothetical 51.8 kDa protein (TR\|Q9LFB4), probable glycosyl | Q91598 (Q91598) Probable glycosyl | 159 | 1.00E−40 |
| | transferase (TR\|Q91598); unknown protein {Arabidopsis thaliana} | transferase. | | |
| TC36241 | Anthocyanin 5-O-glucosyltransferase (TR\|Q9SBQ2); anthocyanin | Q9LTH2 (Q9LTH2) UDP-glycose: flavonoid | 167 | 4.00E−43 |
| | 5-O-glucosyltransferase {Petunia x hybrida} | glycosyltransferase-like. | | |
| TC36278 | T16E15.2 protein (TR\|Q9LMFO); Strong similarity to | Q9LTH2 (Q9LTH2) UDP-glycose: flavonoid | 233 | 4.00E−63 |
| | UDP-glucose glucosyltransferase {Arabidopsis thaliana} (gb\| | glycosyltransferase-like. | | |
| | AB016819) | | | |
| TC36355 | UDP-glycose: flavonoid glycosyltransferase (TR\|Q9ZWQ5) | TC29726.1 [347.1044.4.1044 Fragment_N] | 303 | 2.00E−84 |
| | | Tigr: UDP-glycose: flavono . . . | | |
| TC36367 | T5I8.7 PROTEIN (HYPOTHETICAL 46.3 kDa PROTEIN) | O05384 (O05384) DNA for | 41 | 4.00E−05 |
| | (TR\|Q9SA77); Strong similarity to putative UDP-galactose-4- | glycosyltransferase, lytic transglycosyl . . . | | |
| | epimerase (F1913.8) {Arabidopsis thaliana} (GP\|3033381) | | | |
| TC36466 | Similarity to glycosyl transferase (TR\|Q9A4H4), hypothetical | Q9A4H4 (Q9A4H4) Glycosyl transferase, | 80 | 8.00E−17 |
| | 49.6 kDa protein (TR\|Q9LFQ0); putative protein {Arabidopsis | putative. | | |
| | thaliana} (PIR\|T51450\|T51450) | | | |
| TC36484 | UDP-glycose: flavonoid glycosyltransferase (TR\|Q9ZWQ5); UDP- | TC36484.1 [271.1081 .1080.268 Fragment_C] | 488 | e−140 |
| | glycose: flavonoid glycosyltransferase {Vigna mungo} | Tigr: UDP-glycose: flavo . . . | | |
| TC36569 | Putative glucosyltransferase {Arabidopsis thaliana}, F316.2 | 60533.m00038·0MDC8.15#At3g16520 | 100 | 6.00E−37 |
| | protein (TR\|O48676); unnamed protein product {Brassica napus} | putative glucosyltransferase simi . . . | | |
| TC36593 | UDP-glycose: flavonoid glycosyltransferase (TR\|Q9ZWQ5) | TC29726.1 [347.1044.4.1044 Fragment_N] | 209 | e−109 |
| | | Tigr: UDP-glycose: flavono . . . | | |
| TC36598 | DIGALACTOSYLDIACYLGLYCEROL SYNTHASE (TR\| | Q97BD4 (Q97BD4) Glycosyl transferase. | 40 | 4.00E−05 |
| | Q9S7D1); digalactosyldiacylglycerol synthase {Arabidopsis | | | |
| | thaliana} (GP\|5354160\|gb\|AAD42379.1\| | | | |
| | AF149842_1) | | | |
| TC36622 | Immediate-early salicylate-induced glucosyltransferase | TC29726.1 [347.1044.4.1044 Fragment_N] | 130 | 3.00E−61 |
| | (TR\|P93365); betanidin−5-O-glucosyltransferase {Dorotheanthus | Tigr: UDP-glycose: flavono . . . | | |
| | bellidiformis} | | | |
| TC36660 | Putative anthocyanidin-3-glucoside rhamnosyltransferase | TC29726.1 [347.1044.4.1044 Fragment_N] | 62 | 8.00E−12 |
| | (TR\|Q9ZQ54) | Tigr: UDP-glycose: flavono . . . | | |
| TC36716 | F14J16.9 (TR\|Q9LG28), weak similarity to glycosyltransferase | Q97J01 (Q97J01) Glycosyltransferase, | 46 | 1.00E−06 |
| | (TR\|Q97J01); F14J16.9 {Arabidopsis thaliana} | involved in cell wall bioge . . . | | |
| TC36751 | UDP-GLUCOSE GLUCOSYLTRANSFERASE (T16E15.3 | Q9LTH2 (Q9LTH2) UDP-glycose: flavonoid | 90 | 3.00E−20 |
| | PROTEIN) (TR\|Q9ZWJ3); UDP-glucose glucosyltransferase | glycosyltransferase-like. | | |
| | {Arabidopsis thaliana} | | | |
| | (GP\|9392679\|gb\|AAF87256.1\|AC0685623_AC068562) | | | |
| TC36984 | UDP-GLUCOSE GLUCOSYLTRANSFERASE (T16E15.3 | Q9LTH2 (Q9LTH2) UDP-glycose: flavonoid | 90 | 4.00E−20 |
| | PROTEIN) (TR\|Q9ZWJ3); UDP-glucose glucosyltransferase | glycosyltransferase-like. | | |
| | {Arabidopsis thaliana} | | | |
| | (GP\|9392679\|gb\|AAF87256.1\|AC068562_3\|AC068562) | | | |
| TC37075 | Limonoid UDP-glucosyltransferase (EC 2.4.1.210) | Q9ZWQ5 (Q9ZWQ5) UDP-glycose: flavonoid | 45 | 1.00E−06 |
| | (SP\|Q9MB73\|LGT_CITUN); LIMONOID UDP- | glycosyltransferase. | | |
| | GLUCOSYLTRANSFERASE (EC 2.4.1.210) (LIMONOID | | | |
| | GLUCOSYLTRANSFERASE) (LIMONOID GTASE) | | | |
| TC37114 | Putative endoxyloglucan glycosyltrase {Arabidopsis thaliana}, | 42618.m00036#T9F8.4#At2g06850 putative | 142 | 3.00E−36 |
| | T10O24.17 (TR\|Q9XIJ7); T10O24.17 {Arabidopsis thaliana} | endoxyloglucan glycosyltr . . . | | |
| | (PIR\|A86239\|A86239) | | | |
| TC37182 | UDP-glucose: sterol glucosyltransferase (EC 2.4.1.173) | Q9AFC6 (Q9AFC6) Glycosyltransferase GtfE. | 70 | 4.00E−14 |
| | (TR\|O22678); unnamed protein product | | | |
| | (GP\|2462911\|emb\|CAB06081.1\|Z83832) | | | |
| TC37231 | Flavonol 3-O-glucosyltransferase-like protein (TR\|Q9LK73); UDP- | TC3723.1.1 [261.900.43.825]Tigr: UDP- | 528 | e−152 |
| | glycose: flavonoid glycosyltransferase {Glycine max} | glycose: flavonoid glycosylt . . . | | |
| TC37275 | Gb\|AAC34345.1 (TR\|Q9LSB1); strong similarity to unknown | 60742.m00l38#F14C21.47#Atlg54940 | 167 | 1.00E−65 |
| | protein, MVE11.2 {Arabidopsis thaliana} (gb\|AAC34345.1) | hypothetical protein contains s . . . | | |
| TC37332 | UDP-glucose glucosyltransferase (TR\|P93789) | Q9ZWQ5 (Q9ZWQ5) UDP-glycose: flavonoid | 89 | 4.00E−20 |
| | | glycosyltransferase. | | |
| TC37448 | T10M13.14 (PREDICTED GLYCOSYL TRANSFERASE) | TC37448.1 [154.835.834.373 Fragment_C] | 340 | 1.00E−95 |
| | (TR\|O04253); predicted glycosyl transferase {Arabidopsis | Tigr: predicted glycosyl . . . | | |
| | thaliana} (GP\|2104536\|gb\| | | | |
| | AAC78704.1\|\|AF001308) | | | |
| TC37496 | Putative ribophorin I (TR\|Q9SFX3); putative ribophorin I | TC37496.1 [198.679.618.25]Tigr: putative | 337 | 1.00E−94 |
| | (dolichyl-diphosphooligosaccharide-protein glycosyltransferase) | ribophorin l (dolichyl . . . | | |
| TC37522 | Limonoid UDP-glucosyltransferase (EC 2.4.1.210) | Q9LTH2 (Q9LTH2) UDP-glycose: flavonoid | 159 | 4.00E−41 |
| | (SP\|Q9MB73\|LGT_CITUN); LIMONOID UDP- | glycosyltransferase-like. | | |
| | GLUCOSYLTRANSFERASE (EC 2.4.1.210) (LIMONOID | | | |
| | GLUCOSYLTRANSFERASE) (LIMONOID GTASE) | | | |
| TC37668 | UDP-glucose: salicylic acid glucosyltransferase (TR\|Q9M6E7) | Q9LTH2 (Q9LTH2) UDP-glycose: flavonoid | 40 | 1.00E−05 |
| | | glycosyltransferase-like. | | |
| TC37709 | Putative AUX1-like permease (TR\|Q9FEL8); putative AUX1-like | Q97FZ6 (Q97FZ6) Glycosyltransferase. | 40 | 8.00E−05 |
| | permease | | | |
| TC38000 | Flavonol 3-O-glucosyltransferase-like (TR\|Q9LVW3); flavonol 3- | O86304 (O86304) Macrolide glycosyl | 40 | 2.00E−05 |
| | O-glucosyltransferase-like {Arabidopsis thaliana} | transferase. | | |
| TC38091 | Hypothetical 64.2 kDa protein (TR\|Q9FWA4), glycosyl | 60500.m00065#MJL12.8#At3g25140 glycosyl | 185 | 5.00E−49 |
| | transferase {Arabidopsis thaliana}; unknown protein; 9779-11709 | transferase, putative co . . . | | |
| | {Arabidopsis thaliana} | | | |
| TC38234 | Putative glucosyltransferase (TR\|Q9C9B0) | Q9ZWQ5 (Q9ZWQ5) UDP-glycose: flavonoid | 107 | 2.00E−25 |
| | | glycosyltransferase. | | |
| TC38671 | F18O14.2 (TR\|Q9LN68), putative glycosyl transferase | 67323.m00008#F26K9.90#At3g62660 | 196 | 4.00E−52 |
| | {Arabidopsis thaliana}; F18O14.2 {Arabidopsis thaliana} | putative protein glycosyl transf . . . | | |
| TC38697 | F3I6.10 protein (TR\|O48684), putative glycosyl transferase | 50826.m00113#F3I6.10#At1g24170 putative | 94 | 1.00E−21 |
| | {Arabidopsis thaliana} | glycosyl transferase sim . . . | | |
| TC38956 | UDP-glycose: flavonoid glycosyltransferase (TR\|Q9ZWQ3) | Q9ZWQ3 (Q9ZWQ3) UDP-glycose: flavonoid | 118 | 7.00E−29 |
| | | glycosyltransferase (Fragm . . . | | |
| TC39111 | Anthocyanidin-3-glucoside rhamnosyltransferase (TR\|Q9LSM0); | Q9LTH2 (Q9LTH2) UDP-glycose: flavonoid | 108 | 9.00E−26 |
| | putative anthocyanidin-3-glucoside rhamnosyltransferase | glycosyltransferase-like. | | |
| | {Arabidopsis thaliana} (PIR\|D84614\|D84614) | | | |
| TC39305 | Arbutin synthase (TR\|Q9AR73); Similar to UTP-glucose | 60533.m00038#MDC8.15#At3g16520 | 244 | 6.00E−66 |
| | glucosyltransferases {Arabidopsis thaliana} | putative glucosyltransferase simi . . . | | |
| | (PIR\|G86144\|G86144) | | | |
| TC39353 | UDP-glucose 4-epimerase (EC 5.1.3.2) | O05384 (O05384) DNA for | 47 | 9.00E−07 |
| | (SP\|Q43070\|GAE1_PEA); UDP-galactose-4-epimerase {Pisum | glycosyltransferase, lytic transglycosyl . . . | | |
| | sativum} | | | |
| TC39421 | Glucosyltransferase-like protein (TR\|Q9FNI7); | Q97J01 (Q97J01) Glycosyltransferase, | 84 | 6.00E−18 |
| | glucosyltransferase-like protein {Arabidopsis thaliana} | involved in cell wall bioge . . . | | |
| TC39522 | Arbutin synthase (TR\|Q9AR73); arbutin synthase {Rauvolfia | 60533.m00038#MDC8.15#At3g16520 | 255 | 1.00E−69 |
| | serpentina} | putative glucosyltransferase simi . . . | | |
| TC39526 | Endoxyloglucan transferase (TR\|O65734); | 42618.m00036#T9F8.4#At2g06850 putative | 141 | 6.00E−36 |
| | endoxyloglucan transferase {Cicer arietinum} | endoxyloglucan glycosyltr . . . | | |
| TC39539 | CELLULOSE SYNTHASE CATALYTIC SUBUNIT (TR\| | Q9RDB5 (Q9RDB5) Putative glycosyl | 54 | 7.00E−09 |
| | O48946); unnamed protein product {Arabidopsis thaliana} | transferase. | | |
| | (GP\|4049343\|emb\|CAA22568.1\|\|AL034567) | | | |
| TC39629 | Arbutin synthase (TR\|Q9AR73); arbutin synthase {Rauvolfia | 60533.m00038#MDC8.15#At3g16520 | 272 | 7.00E−75 |
| | serpentina} | putative glucosyltransferase simi . . . | | |
| TC39630 | Arbutin synthase (TR\|Q9AR73); arbutin synthase {Rauvolfia | 60533.m00038#MDC8.15#At3g16520 | 274 | 2.00E−75 |
| | serpentina} | putative glucosyltransferase simi . . . | | |
| TC39706 | Putative O-linked GIcNAc transferase (TR\|Q9M8Y0); putative O- | Q97E12 (Q97E12) Glycosyltransferase fused | 42 | 6.00E−05 |
| | linked GIcNAc transferase {Arabidopsis thaliana} | to TPR-repeat domain. | | |
| TC39745 | T5I8.7 PROTEIN (HYPOTHETICAL 46.3 kDa PROTEIN) | O05384 (O05384) DNA for | 49 | 1.00E−07 |
| | (TR\|Q9SA77), weak similarity to glycosyltransferase | glycosyltransferase, lytic transglycosyl . . . | | |
| | (TR\|O05384); Strong similarity to F19I3.8 GP\|3033381, putative | | | |
| | UDP-galactose-4-epimerase {Arabidopsis thaliana} | | | |
| TC39749 | UDP-glucosyltransferase HRA25 (TR\|Q9FUJ6) | Q9LTH2 (Q9LTH2) UDP-glycose: flavonoid | 45 | 1 .00E−06 |
| | | glycosyltransferase-like. | | |
| TC39750 | UDP-glucosyltransferase HRA25 (TR\|Q9FUJ6) | Q9LTH2 (Q9LTH2) UDP-glycose: flavonoid | 87 | 3.00E−19 |
| | | glycosyltransferase-like. | | |
| TC39765 | Endoxyloglucan transferase (TR\|Q9AT33); endoxyloglucan | 42618.m00036#T9F8.4#At2g06850 putative | 188 | 1.00E−49 |
| | transferase {Daucus carota} | endoxyloglucan glycosyltr . . . | | |
| TC39766 | Endoxyloglucan transferase (TR\|Q9AT33); endoxyloglucan | 42618.m00036#T9F8.4#At2g06850 putative | 181 | 8.00E−48 |
| | transferase {Daucus carota} | endoxyloglucan glycosyltr . . . | | |
| TC39767 | Endoxyloglucan transferase (TR\|Q9SEB1) | Q9ZVK1 (Q9ZVK1) Putative endoxyloglucan | 80 | 3.00E−17 |
| | | glycosyltransferase. | | |
| TC39837 | CELLULOSE SYNTHASE CATALYTIC SUBUNIT | Q9RDB5 (Q9RDB5) Putative glycosyl | 51 | 4.00E−08 |
| | (TR\|Q9SWW6); cellulose synthase catalytic subunit (IRX3) | transferase. | | |
| | {Arabidopsis thaliana} (GP\|5230423\|gb\|AAD40885.1\|AF091713) | | | |
| TC39869 | MGDG synthase type A (TR\|Q9FZL4); MGDG synthase type A | YPFP_BACSU (P54166) Putative glycosyl | 44 | 5.00E−08 |
| | {Glycine max} | transferase ypfP (EC 2.—.— . . . | | |
| TC39874 | Putative glycosyl transferase, Emb\|CAB71043.1 (TR\|Q9LSG3); | 60500.m00065#MJL12.8#At3g25140 glycosyl | 891 | 0 |
| | similar to unknown protein, MJL12.8 {Arabidopsis thaliana} | transferase, putative co . . . | | |
| | (emb\|CAB71043.1) | | | |
| TC39980 | Xyloglucan endo-transglycosylase-like protein (TR\|Q9XHM8); | Q9ZVK1 (Q9ZVK1) Putative endoxyloglucan | 300 | 3.00E−83 |
| | xyloglucan endo-transglycosylase-like protein | glycosyltransferase. | | |
| TC40058 | Glucosyl transferase (TR\|P93709); cold-induced glucosyl | Q9LTH2 (Q9LTH2) UDP-glycose: flavonoid | 128 | 2.00E−31 |
| | transferase {Solanum sogarandinum} | glycosyltransferase-like. | | |
| TC40067 | Monogalactosyldiacylglycerol synthase (EC 2.4.1.46) | YPFP_BACSU (P54166) Putative glycosyl | 88 | 4.00E−19 |
| | (TR\|O82730); monogalactosyldiacylglycerol synthase | transferase ypfP (EC 2.—.— . . . | | |
| | {Arabidopsis thaliana} (PIR\|T52269\|T52269) | | | |
| TC40069 | Putative galactinol synthase (EC 2.4.1.123) (TR\|Q9XGG4); | 60742.m00138#F14C21.47#At1g54940 | 76 | 9.00E−16 |
| | putative galactinol synthase {Pisum sativum} | hypothetical protein contains s . . . | | |
| TC40162 | Putative flavonol 3-O-glucosyltransferase (TR\|O82383); putative | 60533.m00038#MDC8.15#At3g16520 | 84 | 2.00E−18 |
| | flavonol 3-O-glucosyltransferase {Arabidopsis thaliana} | putative glucosyltransferase simi . . . | | |
| | (PIR\|F84699\|F84699) | | | |
| TC40209 | Glycosyl transferase 1 (TR\|Q9LE59), 68 kDa protein | Q9LE59 (Q9LE59) Like glycosyl transferase 1. | 449 | e−128 |
| | (TR\|Q9M3Y6); 68 kDa protein {Cicer arietinum} | | | |
| TC40211 | Flavonol 3-O-glucosyltransferase-like protein (TR\|Q9LK73); | TC37231.1 [261.900.43.825] Tigr: UDP- | 287 | 1.00E−79 |
| | flavonol 3-O-glucosyltransferase-like protein {Arabidopsis | glycose: flavonoid glycosylt . . . | | |
| | thaliana} (GP\|14335152\|gb\|AAK59856.1) | | | |
| TC40408 | Putative glycosyl transferase, hypothetical 64.2 kDa protein | 60500.m00065#MJL12.8#At3g25140 glycosyl | 536 | e−154 |
| | (TR\|Q9FWA4); unknown protein; 9779-11709 {Arabidopsis | transferase, putative co . . . | | |
| | thaliana} | | | |
| TC40431 | Phenylpropanoid: glucosyltransferase 1 (TR\|Q9AT54); UDP- | Q9ZWQ5 (Q9ZWQ5) UDP-glycose: flavonoid | 246 | 5.00E−67 |
| | glucose: flavonoid 7-O-glucosyltransferase {Scutellaria | glycosyltransferase. | | |
| | baicalensis} (GP\|5763524\|dbj\|BAA83484.1) | | | |
| TC40468 | F14J16.9 (TR\|Q9LG28), weak similarity to glycosyltransferases | Q97IS9 (Q97IS9) Glycosyltransferases, | 40 | 2.00E−05 |
| | (TR\|Q97IS9); F14J16.9 {Arabidopsis thaliana} | involved in cell wall biog . . . | | |
| TC40600 | Putative flavonol glucosyltransferase (TR\|Q9M156); putative | TC28828.1 [207.865.865.245 Fragment_C] | 93 | 1.00E−33 |
| | flavonol glucosyltransferase {Arabidopsis thaliana} | Tigr: UDP-glycose: flavono . . . | | |
| | (GP\|13430700\|gb\|AAK25972.1\|AF360262_ 1) | | | |
| TC40745 | Indole-3-acetate beta-glucosyltransferase-like protein | Q9LTH2 (Q9LTH2) UDP-glycose: flavonoid | 41 | 2.00E−05 |
| | (TR\|Q9LVF0); indole-3-acetate beta-glucosyltransferase like | glycosyltransferase-like. | | |
| | protein {Arabidopsis thaliana} (GP\|2244905\|emb\|CAB1032) | | | |
| TC40787 | Cellulose synthase isolog (TR\|O22990) | Q97IS9 (Q97IS9) Glycosyltransferases, | 39 | 5.00E−05 |
| | | involved in cell wall biog . . . | | |
| TC40799 | UDP-glycose: flavonoid glycosyltransferase (TR\|Q9ZWQ5); UDP- | TC40799.1 [287.862.1.861 Fragment_I] Tigr: | 597 | e−173 |
| | glycose: flavonoid glycosyltransferase {Vigna mungo} | UDP-glycose: flavonoid . . . | | |
| TC40859 | T16E15.2 protein (TR\|Q9LMF0); Putative UDP-glucose | Q9LTH2 (Q9LTH2) UDP-glycose: flavonoid | 179 | 6.00E−47 |
| | glucosyltransferase {Arabidopsis thaliana} (PIR\|H86356\|H86356) | glycosyltransferase-like. | | |
| TC40871 | MGDG synthase type A (TR\|Q9FZL4); MGDG synthase type A | YPFP_BACSU (P54166) Putative glycosyl | 68 | 1.00E−13 |
| | {Glycine max} | transferase ypfP (EC 2.—.— . . . | | |
| TC41438 | UDP-GLUCOSE GLUCOSYLTRANSFERASE (T16E15.3 | Q9LTH2 (Q9LTH2) UDP-glycose: flavonoid | 68 | 1.00E−13 |
| | PROTEIN) (TR\|Q9ZWJ3); UDP-glucose glucosyltransferase | glycosyltransferase-like. | | |
| | {Arabidopsis thaliana} | | | |
| | (GP\|9392679\|gb\|AAF87256.1\|AC068562_ 3\|AC068562) | | | |
| TC41440 | UTP-glucose glucosyltransferase (TR\|Q9LSY8) | Q9ZWQ4 (Q9ZWQ4) UDP-glycose: flavonoid | 57 | 1.00E−10 |
| | | glycosyltransferase (Fragm . . . | | |
| TC41557 | HYPOTHETICAL 53.1 kDa PROTEIN (TR\|O22775); putative | TC41557.1 [186.810.809.252 Fragment_C] | 399 | e−113 |
| | golgi glycosyltransferase {Arabidopsis thaliana} | Tigr: putative golgi glyc . . . | | |
| | (GP\|3193287\|gb\|AAC19271.1\|\|AF069298) | | | |
| TC41607 | UDP-glucose: sterol glucosyltransferase (TR\|Q9M8Z7); UDP- | Q9RMP0 (Q9RMP0) Putative | 43 | 5.00E−06 |
| | glucose: sterol glucosyltransferase {Arabidopsis thaliana} | glycosyltransferase. | | |
| TC41869 | Xyloglucan endotransglycosylase XET1 (EC 2.4.1.207) | 42618.m00036#T9F8.4#At2g06850 putative | 172 | 3.00E−45 |
| | (TR\|Q9LLC3); xyloglucan endotransglycosylase XET1 | endoxyloglucan glycosyltr . . . | | |
| | {Asparagus officinalis} | | | |
| TC41956 | T10B10.8 protein (TR\|Q22375) | 60742.m00138#F14C21.47#At1g54940 | 79 | 7.00E−17 |
| | | hypothetical protein contains s . . . | | |
| TC41975 | UDP-glycose: flavonoid glycosyltransferase (TR\|Q9ZWQ5); UDP- | TC41975.1 [146.566.3.440 Fragment_C] Tigr: | 273 | 9.00E−76 |
| | glycose: flavonoid glycosyltransferase {Vigna mungo} | UDP-glycose: flavonoid . . . | | |
| TC41977 | UDP-glucose glucosyltransferase (TR\|P93789); immediate-early | Q9ZWQ5 (Q9ZWQ5) UDP-glycose: flavonoid | 127 | 7.00E−32 |
| | salicylate-induced glucosyltransferase {Nicotiana tabacum} | glycosyltransferase. | | |
| | (GP\|1685005\|gb\|AAB36653.1) | | | |
| TC41993 | T16E15.2 protein (TR\|Q9LMF0), weak similarity to UDP- | Q9LTH2 (Q9LTH2) UDP-glycose: flavonoid | 62 | 5.00E−12 |
| | glycose: flavonoid glycosyltransferase-like protein (TR\|Q9LTH2) | glycosyltransferase-like. | | |
| TC42052 | T16E15.1 protein (TR\|Q9LMF1); Strong similarity to UDP-glucose | Q9LTH2 (Q9LTH2) UDP-glycose: flavonoid | 142 | 3.00E−36 |
| | glucosyltransferase {Arabidopsis thaliana} (gb\|AB016819) | glycosyltransferase-like. | | |
| TC42457 | Weak similarity to glycosyl transferases-like protein (TR\|Q9LSB5) | Q9LSB5 (Q9LSB5) Glycosyl transferases-like | 39 | 3.00E−05 |
| | | protein. | | |
| TC42628 | HYPOTHETICAL 20.8 kDa PROTEIN (TR\|Q9SMM4), weak | O96196 (O96196) Glycosyl transferase (novel | 39 | 8.00E−05 |
| | similarity to glycosyl transferase (novel euk. family) (TR\|O96196); | euk. family). | | |
| | putative protein {Arabidopsis thaliana} | | | |
| | (GP\|7268616\|emb\|CAB78825.1\|\|AL161548) | | | |
| TC42667 | UDP-GLUCOSE GLUCOSYLTRANSFERASE (T16E15.3 | Q9LTH2 (Q9LTH2) UDP-glycose: flavonoid | 52 | 7.00E−09 |
| | PROTEIN) (TR\|Q9ZWJ3) | glycosyltransferase-like. | | |
| TC42735 | T16E15.5 protein (TR\|Q9LME8); Strong similarity to UDP- | Q9LTH2 (Q9LTH2) UDP-glycose: flavonoid | 77 | 2.00E−16 |
| | glucose glucosyltransferase {Arabidopsis thaliana} | glycosyltransferase-like. | | |
| | (gb\|AB016819) | | | |
| TC42877 | Glucosyltransferase-like protein (TR\|Q9LXV0) | TC29726.1 [347.1044.4.1044 Fragment_N] | 56 | 4.00E−10 |
| | | Tigr: UDP-glycose: flavono . . . | | |
| AI974832 | Limonoid UDP-glucosyltransferase (EC 2.4.1.210) | TC36484.1 [271.1081.1080.268 Fragment_C] | 39 | 4.00E−05 |
| | (SP\|Q9MB73\|LGT_CITUN) | Tigr: UDP-glycose: flavo . . . | | |
| AL365925 | Similarity to UDP-glycose: flavonoid glycosyltransferase-like | TC29557.1 [231.1071.1070.378 Fragment_C] | 72 | 2.00E−15 |
| | protein | Tigr: UDP-glycose: flavo . . . | | |
| AL367345 | T16E15.5 protein (TR\|Q9LME8), similarity to UDP- | Q9LTH2 (Q9LTH2) UDP-glycose: flavonoid | 82 | 6.00E−18 |
| | glycose: flavonoid glycosyltransferase-like protein (TR\|Q9LTH2) | glycosyltransferase-like. | | |
| AL367433 | Weak similarity to UDP-glycose: flavonoid glycosyltransferase | Q9ZWQ5 (Q9ZWQ5) UDP-glycose: flavonoid | 40 | 1.00E−05 |
| | (TR\|Q9ZWQ5) | glycosyltransferase. | | |
| AL367828 | F3F9.19 (TR\|Q9M9E7), weak similarity to UDP-glycose: | Q9LTH2 (Q9LTH2) UDP-glycose: flavonoid | 54 | 4.00E−10 |
| | flavonoid glycosyltransferase-like protein (TR\|Q9LTH2) | glycosyltransferase-like. | | |
| AL367875 | Similarity to UDP-glycose: flavonoid glycosyltransferase-like | TC29557.1 [231.1071.1070.378 Fragment_C] | 92 | 3.00E−21 |
| | protein | Tigr: UDP-glycose: flavo . . . | | |
| AL368568 | UDP-glycose: flavonoid glycosyltransferase (TR\|Q9ZWQ5) | TC40799.1 [287.862.1.861 Fragment_I] Tigr: | 141 | 4.00E−36 |
| | | UDP-glycose: flavonoid . . . | | |
| AL368569 | UDP-glycose: flavonoid glycosyltransferase (TR\|Q9ZWQ5) | TC29557.1 [231.1071.1070.378 Fragment_C] | 182 | 2.00E−48 |
| | | Tigr: UDP-glycose: flavo . . . | | |
| AL369284 | Hypothetical 60.3 kDa protein (TR\|Q9LXS3), weak similarity to | Q9LE59 (Q9LE59) Like glycosyl transferase 1. | 62 | 5.00E−12 |
| | glycosyl transferase 1 (TR\|Q9LE59) | | | |
| AL370080 | T16E15.2 protein (TR\|Q9LMF0), similarity to UDP- | Q9LTH2 (Q9LTH2) UDP-glycose: flavonoid | 102 | 2.00E−24 |
| | glycose: flavonoid glycosyltransferase-like protein (TR\|Q9LTH2) | glycosyltransferase-like. | | |
| AL372207 | Flavonol 3-O-glucosyltransferase-like protein (TR\|Q9FN26) | Q9ZWQ4 (Q9ZWQ4) UDP-glycose: flavonoid | 65 | 6.00E−13 |
| | | glycosyltransferase (Fragm . . . | | |
| AL375348 | Arbutin synthase (TR\|Q9AR73) | TC42093.1 [118.648.1.354 Fragment_C] Tigr: | 45 | 7.00E−09 |
| | | UDP-glycose: flavonoid . . . | | |
| AL376164 | HYPOTHETICAL 39.5 kDa PROTEIN (TR\|Q9SZB0) | 60742.m00138#F14C21.47#At1g54940 | 112 | 3.00E−27 |
| | | hypothetical protein contains s . . . | | |
| AL377152 | At2g20810 protein (TR\|Q9SKT6), glycosyl transferase | 60500.m00065#MJL12.8#At3g25140 glycosyl | 151 | 3.00E−39 |
| | {Arabidopsis thaliana} | transferase, putative co . . . | | |
| AL378735 | Similarity to UDP-glycose: flavonoid glycosyltransferase-like | TC40799.1 [287.862.1.861 Fragment_I] Tigr: | 84 | 3.00E−19 |
| | protein | UDP-glycose: flavonoid . . . | | |
| AL378962 | Putative ribophorin I homologue (Fragment) (TR\|O49868) | 60052.m00002#F15M4.10#At1g76400 | 201 | 3.00E−54 |
| | | putative ribophorin I (dolichyl- . . . | | |
| AL381855 | UDP RHAMNOSE--ANTHOCYANIDIN-3-GLUCOSIDE | Q9ZWQ4 (Q9ZWQ4) UDP-glycose: flavonoid | 74 | 5.00E−16 |
| | (TR\|Q9T081) | glycosyltransferase (Fragm . . . | | |
| AL385256 | UDP RHAMNOSE--ANTHOCYANIDIN-3-GLUCOSIDE | Q9ZWQ4 (Q9ZWQ4) UDP-glycose: flavonoid | 70 | 1.00E−14 |
| | (TR\|Q9T081) | glycosyltransferase (Fragm . . . | | |
| AL389151 | Weak similarity to glycosyltransferase-like protein (TR\|Q9UGZ8) | Q9UGZ8 (Q9UGZ8) BK282F2.1 (like- | 43 | 3.00E−06 |
| | | glycosyltransferase (KIAAO6O9)) . . . | | |
| AW126073 | Putative anthocyanidin-3-glucoside rhamnosyltransferase | Q9ZWQ5 (Q9ZWQ5) UDP-glycose: flavonoid | 70 | 2.00E−14 |
| | (TR\|Q9ZQ54) | glycosyltransferase. | | |
| AW127509 | UDP-glycose: flavonoid glycosyltransferase (TR\|Q9ZWQ5) | TC29726.1 [347.1044.4.1044 Fragment_N] | 76 | 8.00E−17 |
| | | Tigr: UDP-glycose: flavono . . . | | |
| AW256664 | Limonoid UDP-glucosyltransferase (EC 2.4.1.210) | TC41975.1 [146.566.3.440 Fragment_C] | 84 | 2.00E−18 |
| | (SP\|Q9MB73\|LGT_CITUN); limonoid UDP-glucosyltransferase | Tigr: UDP-glycose: flavonoid . . . | | |
| | {Citrus unshiu} | | | |
| AW257169 | Xyloglucan endotransglycosylase (TR\|Q9FXQ4); endoxyloglucan | 42618.m00036#T9F8.4#At2g06850 putative | 103 | 6.00E−37 |
| | transferase {Cicer arietinum} | endoxyloglucan glycosyltr . . . | | |
| AW268009 | Putative glucosyltransferase (TR\|O64732); putative | Q9LTH2 (Q9LTH2) UDP-glycose: flavonoid | 40 | 2.00E−05 |
| | glucosyltransferase {Arabidopsis thaliana} (PIR\|T00584\|T00584) | glycosyltransferase-like. | | |
| AW299178 | Arbutin synthase (TR\|Q9AR73) | 60533.m00038#MDC8.15#At3g16520 | 47 | 5.00E−08 |
| | | putative glucosyltransferase simi . . . | | |
| AW329526 | F6A14.20 protein (TR\|Q9M9U0), | Q9LF80 (Q9LF80) Putative golgi | 48 | 9.00E−08 |
| | | glycosyltransferase (Alpha galact . . . | | |
| AW329566 | Weak similarity to UDP-glycose: flavonoid glycosyltransferase-like | Q9LTH2 (Q9LTH2) UDP-glycose: flavonoid | 37 | 1.00E−04 |
| | protein (TR\|Q9LTH2) | glycosyltransferase-like. | | |
| AW559311 | Immediate-early salicylate-induced glucosyltransferase | TC36484.1 [271.1081.1080.268 Fragment_C] | 211 | 4.00E−57 |
| | (TR\|P93365) | Tigr: UDP-glycose: flavo . . . | | |
| AW559693 | HYPOTHETICAL 53.1 kDa PROTEIN (TR\|O22775); putative | AW559693.1 [161.608.124.606 Fragment_N] | 289 | 2.00E−80 |
| | golgi glycosyltransferase {Arabidopsis thaliana} | Tigr: putative golgi gly . . . | | |
| | (GP\|3193287\|gb\|AAC19271.1\|\|AF069298) | | | |
| AW560798 | UDPG glucosyltransferase-like protein (TR\|Q9LZD8) | 60533.m00038#MDC8.15#At3g16520 | 68 | 2.00E−14 |
| | | putative glucosyltransferase simi . . . | | |
| AW585026 | ENDOXYLOGLUCAN TRANSFERASE (TR\|Q9SEB0) | Q9ZVK1 (Q9ZVK1) Putative endoxyloglucan | 117 | 1.00E−32 |
| | | glycosyltransferase. | | |
| AW585051 | F18O14.2 (TR\|Q9LN68), similarity to the PREDICTED | O04253 (O04253) T10M13.14 (PREDICTED | 225 | 3.00E−61 |
| | GLYCOSYL TRANSFERASE, T10M13.14 (TR\|O04253); | GLYCOSYL TRANSFERASE). | | |
| | F18O14.2 {Arabidopsis thaliana} | | | |
| AW585334 | Weak similarity to putative glycosyltransferase (TR\|P95720) | P95720 (P95720) Putative glycosyltransferase | 39 | 5.00E−05 |
| | | (Fragment). | | |
| AW586147 | Putative alpha galactosyltransferase (TR\|Q9CA75); putative | Q9LF80 (Q9LF80) Putative golgi | 194 | 5.00E−52 |
| | alpha galactosyltransferase {Arabidopsis thaliana} | glycosyltransferase (Alpha galact . . . | | |
| | (GP\|9989328\|gb\|AAG11078.1\|AC079658_26) | | | |
| AW586859 | Immediate-early salicylate-induced glucosyltransferase | Q9ZWQ5 (Q9ZWQ5) UDP-glycose: flavonoid | 113 | 2.00E−27 |
| | (TR\|P93365) | glycosyltransferase. | | |
| AW684054 | Arbutin synthase (TR\|Q9AR73) | TC37231.1 [261.900.43.825] Tigr: UDP- | 62 | 6.00E−12 |
| | | glycose: flavonoid glycosylt . . . | | |
| AW684227 | UDP-glucose: salicylic acid glucosyltransferase (TR\|Q9M6E7); | Q9LTH2 (Q9LTH2) UDP-glycose: flavonoid | 49 | 4.00E−08 |
| | UDP-glucose: salicylic acid glucosyltransferase {Nicotiana | glycosyltransferase-like. | | |
| | tabacum} | | | |
| AW684307 | PUTATIVE XYLOGLUCAN ENDOTRANSGLYCOSYLASE | 42618.m00036#T9F8.4#At2g06850 putative | 53 | 9.00E−10 |
| | (TR\|Q9ZR10) | endoxyloglucan glycosyltr . . . | | |
| AW684612 | At2g20810 protein (TR\|Q9SKT6) {Arabidopsis thaliana} | 66314.m00033#F25I16.8#At1g18580 | 92 | 3.00E−21 |
| | | hypothetical protein contains Pf . . . | | |
| AW687720 | Glucosyltransferase-like protein (TR\|Q9LXV0) | TC29557.1 [231.1071.1070.378 Fragment_C] | 87 | 2.00E−20 |
| | | Tigr: UDP-glycose: flavo . . . | | |
| AW687987 | T12J13.3 protein (TR\|Q9SS69), putative glycosyl transferase | Q9A4H4 (Q9A4H4) Glycosyl transferase, | 53 | 2.00E−09 |
| | (TR\|Q9A4H4); hypothetical protein {Arabidopsis thaliana} | putative. | | |
| AW690449 | Putative glucosyl transferase (TR\|Q9ZQ95); putative glucosyl | Q9ZWQ5 (Q9ZWQ5) UDP-glycose: flavonoid | 84 | 2.00E−18 |
| | transferase {Arabidopsis thaliana} | glycosyltransferase. | | |
| AW695243 | UDP-glycose: flavonoid glycosyltransferase | AW695243.1 [213.643.641.3 Fragment_I] | 315 | 3.00E−88 |
| | | Tigr: UDP-glycose: flavonoi . . . | | |
| AW695272 | Putative xyloglucan endo-transglycosylase (TR\|Q9SJL9); putative | 42618.m00036#T9F8.4#At2g06850 putative | 105 | 4.00E−25 |
| | xyloglucan endo-transglycosylase {Arabidopsis thaliana} | endoxyloglucan glycosyltr . . . | | |
| AW695874 | N-acetylglucosaminyltransferase I (EC 2.4.1.101) (TR\|Q9XGM8); | Q9SVG1 (Q9SVG1) Glycosyltransferase like | 160 | 8.00E−42 |
| | BETA-1,2-N-ACETYLGLUCOSAMINYLTRANSFERASE I | protein (Fragment). | | |
| | {Arabidopsis thaliana} | | | |
| AW696207 | Putative anthocyanidin-3-glucoside rhamnosyltransferase | TC29726.1 [347.1044.4.1044 Fragment_N] | 66 | 3.00E−13 |
| | (TR\|Q9ZQ54); putative anthocyanidin-3-glucoside | Tigr: UDP-glycose: flavono . . . | | |
| | rhamnosyltransferase {Arabidopsis thaliana} | | | |
| AW774532 | N-acetylglucosaminyltransferase I (EC 2.4.1.101) (TR\|Q9XGM8); | Q9SZM4 (Q9SZM4) | 113 | 2.00E−27 |
| | N-acetylglucosaminyltransferase I {Arabidopsis | GLYCOSYLTRANSFERASE LIKE | | |
| | thaliana} (PIR\|JC7084\|JC7084) | PROTEIN. | | |
| AW775420 | Anthocyanidin-3-glucoside rhamnosyltransferase-like | TC29726.1 [347.1044.4.1044 Fragment_N] | 54 | 2.00E−09 |
| | (TR\|Q9LTA3) | Tigr: UDP-glycose: flavono . . . | | |
| AW775803 | Putative glucosyl transferase (TR\|Q9ZQ99); putative glucosyl | TC29726.1 [347.1044.4.1044 Fragment_N] | 112 | 5.00E−27 |
| | transferase {Arabidopsis thaliana} | Tigr: UDP-glycose: flavono . . . | | |
| AW775814 | UDP-glycose: flavonoid glycosyltransferase-like (TR\|Q9LTH2) | Q9LTH2 (Q9LTH2) UDP-glycose: flavonoid | 36 | 7.00E−05 |
| | | glycosyltransferase-like. | | |
| AW776615 | T16E15.2 protein (TR\|Q9LMF0); Strong similarity to UDP-glucose | Q9LTH2 (Q9LTH2) UDP-glycose: flavonoid | 160 | 1.00E−41 |
| | glucosyltransferase (Arabidopsis thaliana) | glycosyltransferase-like. | | |
| BE203403 | Mannosyltransferase, putative (TR\|Q9LPN6); | Q97F42 (Q97F42) Glycosyltransferase. | 47 | 1.00E−07 |
| | mannosyltransferase, putative {Arabidopsis thaliana} | | | |
| BE203634 | Putative xyloglucan endo-transglycosylase (TR\|Q9SJL9); putative | 42618.m00036#T9F8.4#At2g06850 putative | 68 | 6.00E−14 |
| | xyloglucan endo-transglycosylase {Arabidopsis thaliana} | endoxyloglucan glycosyltr . . . | | |
| BE249479 | UDP-glucose: salicylic acid glucosyltransferase (TR\|Q9M6E7) | Q9LTH2 (Q9LTH2) UDP-glycose: flavonoid | 40 | 1.00E−05 |
| | | glycosyltransferase-like. | | |
| BE317350 | Weak similarity to UDP-glycose: flavonoid glycosyltransferase-like | TC29726.1 [347.1044.4.1044 Fragment_N] | 173 | 2.00E−45 |
| | protein {Arabidopsis thaliana} | Tigr: UDP-glycose: flavono . . . | | |
| BE317583 | Arbutin synthase (TR\|Q9AR73) | 60533.m00038#MDC8.15#At3g16520 | 56 | 3.00E−10 |
| | | putative glucosyltransferase simi . . . | | |
| BE318378 | ETAG-A3 (TR\|Q9SLN9), putative endoxyloglucan | Q9ZVK1 (Q9ZVK1) Putative endoxyloglucan | 79 | 5.00E−17 |
| | glycosyltransferase (TR\|Q9ZVK1) | glycosyltransferase. | | |
| BE320067 | Xyloglucan endotransglycosylase (TR\|Q9FXQ4) | 42618.m00036#T9F8.4#At2g06850 putative | 207 | 3.00E−59 |
| | | endoxyloglucan glycosyltr . . . | | |
| BE321824 | Cellulose synthase catalytic subunit-like protein (TR\|Q9LFL0) | Q9RDB5 (Q9RDB5) Putative glycosyl | 45 | 5.00E−07 |
| | | transferase. | | |
| BE322778 | UDP-glycose: flavonoid glycosyltransferase-like (TR\|Q9LTH2) | Q9LTH2 (Q9LTH2) UDP-glycose: flavonoid | 71 | 4.00E−16 |
| | | glycosyltransferase-like. | | |
| BE323875 | Putative xyloglucan endo-transglycosylase (TR\|Q9SJL9) | Q9ZVK1 (Q9ZVK1) Putative endoxyloglucan | 114 | 8.00E−28 |
| | | glycosyltransferase. | | |
| BE324656 | Putative UDP-glycose (Fragment) (TR\|Q9M3H8) | Q9ZWQ5 (Q9ZWQ5) UDP-glycose: flavonoid | 225 | 4.00E−61 |
| | | glycosyltransferase. | | |
| BE325491 | Putative glucosyltransferase (TR\|O22820) | Q9LTH2 (Q9LTH2) UDP-glycose: flavonoid | 39 | 3.00E−05 |
| | | glycosyltransferase-like. | | |
| BE325650 | Alpha-1,3-mannosyl-glycoprotein (TR\|Q9ST97) | Q9SVG1 (Q9SVG1) Glycosyltransferase like | 130 | 8.00E−33 |
| | | protein (Fragment). | | |
| BE325941 | Weak similarity to putative glycosyl transferase (TR\|Q9A4H4), | Q9A4H4 (Q9A4H4) Glycosyl transferase, | 43 | 4.00E−06 |
| | Gb\|AAF26009.1 (TR\|Q9LlQ3) | putative. | | |
| BE999520 | UDP-glucose 4-epimerase GEPI48 (EC 5.1.3.2) | O05384 (O05384) DNA for | 50 | 2.00E−08 |
| | (SP\|O65781\|GAE2_CYATE) | glycosyltransferase, lytic transglycosyl . . . | | |
| BF004505 | Weak similarity to UDP-glycose: flavonoid glycosyltransferase-like | TC41975.1 [146.566.3.440 Fragment_C]Tigr: | 48 | 3.00E−08 |
| | protein {Arabidopsis thaliana} | UDP-glycose: flavonoid . . . | | |
| BF520536 | UDP-GLUCOSE GLUCOSYLTRANSFERASE (T16E15.3 | Q9LTH2 (Q9LTH2) UDP-glycose: flavonoid | 44 | 8.00E−07 |
| | PROTEIN) (TR\|Q9ZWJ3) | glycosyltransferase-like. | | |
| BF520967 | Flavonol 3-O-glucosyltransferase 2 (EC 2.4.1.91) ( | Q9ZWQ4 (Q9ZWQ4) UDP-glycose: flavonoid | 109 | 2.00E−26 |
| | (SP\|Q40285\|UFO2_MANES) | glycosyltransferase (Fragm . . . | | |
| BF633795 | Glucuronosyl transferase-like protein (TR\|Q9M052) | Q9LTH2 (Q9LTH2) UDP-glycose: flavonoid | 92 | 5.00E−21 |
| | | glycosyltransferase-like. | | |
| BF636776 | Putative anthocyanidin-3-glucoside rhamnosyltransferase | TC29726.1 [347.1044.4.1044 Fragment_N] | 62 | 8.00E−12 |
| | (TR\|Q9ZQ54) | Tigr: UDP-glycose: flavono . . . | | |
| BF640372 | T16E15.5 protein (TR\|Q9LME8), similarity to UDP- | Q9LTH2 (Q9LTH2) UDP-glycose: flavonoid | 79 | 6.00E−17 |
| | glycose: flavonoid glycosyltransferase-like protein (TR\|Q9LTH2) | glycosyltransferase-like. | | |
| BF640780 | UDP-glucose glucosyltransferase-like protein (TR\|Q9LHJ2) | Q9LTH2 (Q9LTH2) UDP-glycose: flavonoid | 43 | 3.00E−06 |
| | | glycosyltransferase-like. | | |
| BF643221 | F9K20.16 protein (TR\|Q9ZV98) | 51028.m00096#F9K20.16#At1g78800 | 143 | 2.00E−36 |
| | | hypothetical protein contains si . . . | | |
| BF644297 | Xyloglucan endotransglycosylase (XET) (TR\|P93671) | Q9ZVK1 (Q9ZVK1) Putative endoxyloglucan | 67 | 6.00E−14 |
| | | glycosyltransferase. | | |
| BF644451 | Limonoid UDP-glucosyltransferase (EC 2.4.1.210) | Q9LTH2 (Q9LTH2) UDP-glycoseflavonoid | 44 | 9.00E−07 |
| | (SP\|Q9MB73\|LGT_CITUN) | glycosyltransferase-like. | | |
| BF646175 | UDP-glycosyltransferase HRA25 (TR\|Q9FUJ6) | Q9LTH2 (Q9LTH2) UDP-glucose: flavonoid | 46 | 3.00E−07 |
| | | glycosyltransferase-like. | | |
| BF646288 | Cellulose synthase isolog (TR\|O22990) | Q97IS9 (Q97IS9) Glycosyltransferases, | 41 | 1.00E−05 |
| | | involved in cell wall biog . . . | | |
| BF650423 | HYPOTHETICAL 75.6 kDa PROTEIN (TR\|Q9SVF8), | Q9LE59 (Q9LE59) Like glycosyl transferase 1. | 273 | 2.00E−75 |
| | similarity to glycosyl transferase 1 (TR\|Q9LE59) | | | |
| BF650554 | Weak similarity to UDP-glycose: flavonoid glycosyltransferase-like | TC29726.1 [347.1044.4.1044 Fragment_N] | 55 | 8.00E−10 |
| | protein {Arabidopsis thaliana} | Tigr: UDP-glycoseflavono . . . | 171 | 8.00E−45 |
| BG449057 | HYPOTHETICAL 38.8 kDa PROTEIN (TR\|Q9S7G2) | 50885.m00110#F20P5.18#At1g70090 | | |
| | | unknown protein similar to putat . . . | | |
| BG449653 | UDPG glucosyltransferase-like protein (TR\|Q9LZD8) | TC40799.1 [287.862.1.861 Fragment_I]Tigr: | 60 | 2.00E−11 |
| | | UDP-glycose: flavonoid . . . | | |
| BG450101 | UDP-glycose: flavonoid glycosyltransferase (TR\|Q9ZWQ5) | TC36484.1 [271.1081.1080.268 Fragment_C] | 284 | 1.00E−79 |
| | | Tigr: UDP-glycose: flavo . . . | | |
| BG450877 | T16E15.2 protein (TR\|Q9LMF0), similarity to UDP- | Q9LTH2 (Q9LTH2) UDP-glycose: flavonoid | 105 | 3.00E−25 |
| | glycose: flavonoid glycosyltransferase-like (TR\|Q9LTH2) | glycosyltransferase-like. | | |
| BG453315 | Xyloglucan endotransglycosylase 1 (TR\|Q9ZRV1) | Q9ZVK1 (Q9ZVK1) Putative endoxyloglucan | 193 | 2.00E−51 |
| | | glycosyltransferase. | | |
| BG581102 | At2g20810 protein (TR\|Q9SKT6) | 66314.m00033#F25I16.8#At1g18580 | 287 | 6.00E−80 |
| | | hypothetical protein contains Pf . . . | | |
| BG582596 | UDP-glucose glucosyltransferase (TR\|P93789) | Q9ZWQ5 (Q9ZWQ5) UDP-glycose: flavonoid | 148 | 6.00E−38 |
| | | glycosyltransferase. | | |
| BG582602 | Sucrose synthase (EC 2.4.1.13) (TR\|Q9XG65) | 084909 (084909) Glycosyltransferase WbpY | 57 | 3.00E−10 |
| | | (Fragment). | | |
| BG584431 | Twi1 protein (TR\|Q43526) | Q9ZWQ5 (Q9ZWQ5) UDP-glycose: flavonoid | 130 | 2.00E−32 |
| | | glycosyltransferase. | | |
| BG584738 | F14J16.9 (TR\|Q9LG28), weak similarity to glycosyltransferases | Q97IS9 (Q97IS9) Glycosyltransferases, | 42 | 7.00E−06 |
| | (TR\|Q97IS9) | involved in cell wall biog . . . | | |
| BG586846 | Arbutin synthase (TR\|Q9AR73) | 60533.m00038#MDC8.15#At3g16520 | 98 | 1.00E−22 |
| | | putative glucosyltransferase simi . . . | | |
| BG586847 | Arbutin synthase (TR\|Q9AR73) | 60533.m00038#MDC8.15#At3g16520 | 54 | 5.00E−18 |
| | | putative glucosyltransferase simi . . . | | |
| BG644402 | Phenylpropanoid: glucosyltransferase 1 (Fragment) (TR\|Q9AT54) | Q9ZWQ5 (Q9ZWQ5) UDP-glycose: flavonoid | 108 | 8.00E−26 |
| | | glycosyltransferase. | | |
| BG644459 | T16E15.2 protein (TR\|Q9LMF0), similarity to UDP- | Q9LTH2 (Q9LTH2) UDP-glycose: flavonoid | 87 | 3.00E−19 |
| | glycose: flavonoid glycosyltransferase-like protein (TR\|Q9LTH2) | glycosyltransferase-like. | | |
| BG645967 | Weak similarity to UDP-glycose: flavonoid glycosyltransferase-like | AW695243.1 [213.643.641.3 Fragment_I] | 39 | 8.00E−05 |
| | protein {Arabidopsis thaliana} | Tigr: UDP-glycose: flavonoi . . . | | |
| BG647358 | Arbutin synthase (TR\|Q9AR73) | 60533.m00038#MDC8.15#At3g16520 | 181 | 4.00E−50 |
| | | putative glucosyltransferase simi . . . | | |
| BI263761 | UDP-glucose: salicylic acid glucosyltransferase (TR\|Q9M6E7) | Q9LTH2 (Q9LTH2) UDP-glycose: flavonoid | 65 | 1.00E−12 |
| | | glycosyltransferase-like. | | |
| BI265578 | UDP-glycose: flavonoid glycosyltransferase (TR\|Q9ZWQ5) | TC29726.1 [347.1044.4.1044 Fragment_N] | 235 | 3.00E−64 |
| | | Tigr: UDP-glycose: flavono . . . | | |
| BI265903 | Arbutin synthase (TR\|Q9AR73) | 60533.m00038#MDC8.15#At3g16520 | 66 | 1.00E−13 |
| | | putative glucosyltransferase simi . . . | | |
| BI266303 | Betanidin-5-O-glucosyltransferase (TR\|Q9SMG6) | Q9ZWQ5 (Q9ZWQ5) UDP-glycose: flavonoid | 51 | 4.00E−09 |
| | | glycosyltransferase. | | |
| BI267731 | UDP-GLUCOSE GLUCOSYLTRANSFERASE (T16E15.3 | Q9LTH2 (Q9LTH2) UDP-glycose: flavonoid | 69 | 5.00E−14 |
| | PROTEIN) (TR\|Q9ZWJ3) | glycosyltransferase-like. | | |
| BI267848 | T16E15.5 protein (TR\|Q9LME8), weak similarity to UDP- | Q9LTH2 (Q9LTH2) UDP-glycose: flavonoid | 56 | 3.00E−10 |
| | glycose: flavonoid glycosyltransferase-like protein (TR\|Q9LTH2) | glycosyltransferase-like. | | |
| Bl268054 | Glucosyltransferase-like protein (TR\|Q9SNB0) | Q9LTH2 (Q9LTH2) UDP-glycose: flavonoid | 200 | 1.00E−53 |
| | | glycosyltransferase-like. | | |
| BI271361 | F1B16.5 protein (TR\|Q9FWT0) | 51942.m00184#F14P1.19#At1g19710 | 259 | 3.00E−88 |
| | | hypothetical protein contains Pf . . . | | |
| BI271396 | F10K1.4 protein (TR\|Q9LML6), similarity to putative | 60533.m00038#MDC8.15#At3g16520 | 73 | 3.00E−15 |
| | glucosyltransferase {Arabidopsis thaliana} | putative glucosyltransferase simi . . . | | |
| BI271442 | Putative flavonol 3-O-glucosyltransferase (TR\|O82383) | 60533.m00038#MDC8.15#At3g16520 | 96 | 5.00E−23 |
| | | putative glucosyltransferase simi . . . | | |
| BI273216 | Putative UDP-glucose glucosyltransferase (TR\|Q9SK82) | Q9LTH2 (Q9LTH2) UDP-glycose: flavonoid | 87 | 6.00E−34 |
| | | glycosyltransferase-like. | | |
| BI308322 | Limonoid UDP-glucosyltransferase (EC 2.4.1.210) (L | 60533.m00038#MDC8.15#At3g16520 | 60 | 3.00E−11 |
| | (SP\|Q9MB73\|LGT_CITUN) | putative glucosyltransferase simi . . . | | |
| BI308477 | Weak similarity to putative glucosyltransferase {Arabidopsis | 60533.m00038#MDC8.15#At3g16520 | 73 | 4.00E−15 |
| | thaliana} | putative glucosyltransferase simi . . . | | |
| BI309064 | T16E15.2 protein (TR\|Q9LMF0), weak similarity to UDP- | Q9LTH2 (Q9LTH2) UDP-glycose: flavonoid | 40 | 1.00E−05 |
| | glycose: flavonoid glycosyltransferase-like protein (TR\|Q9LTH2) | glycosyltransferase-like. | | |
| BI309958 | 68 kDa protein (TR\|Q9M3Y6), glycosyl transferase 1 | Q9LE59 (Q9LE59) Like glycosyl transferase 1. | 366 | e−103 |
| | (TR\|Q9LE59) | | | |
| BI310324 | Anthocyanidin-3-glucoside rhamnosyltransferase (TR\|Q9LSM0) | Q9LTH2 (Q9LTH2) UDP-glycose: flavonoid | 64 | 2.00E−12 |
| | | glycosyltransferase-like. | | |
| BI310822 | Glucosyl transferase, putative (TR\|Q9C768) | Q9LTH2 (Q9LTH2) UDP-glycose: flavonoid | 183 | 1.00E−48 |
| | | glycosyltransferase-like. | | |
| BI311600 | Flavonol 3-O-glucosyltransferase-like (TR\|Q9LVW3) | Q9ZWQ5 (Q9ZWQ5) UDP-glycose: flavonoid | 50 | 4.00E−08 |
| | | glycosyltransferase. | | |

TABLE 4
Medicago truncatula P450 genes with similar expression
pattern to β-amyrin synthase based on cluster analysis

| | |
|---|---|
| TC30190 | Ent-kaurenoic acid hydroxylase (TR\|Q9C5Y3); DWARF3 |
| | {Zea mays} (SP\|Q43246\|C881_MAIZE) |
| TC30649 | Ent-kaurenoic acid hydroxylase (TR\|Q9C5Y3); |
| | CYTOCHROME P450 88A3 (EC 1.14,-,-) {Arabidopsis |
| | thaliana} (GP\|2388581\|gb\|AAB71462) |
| TC31146 | Cytochrome P450-like protein (TR\|Q9LF95) |
| TC31441 | (S)-N-methylcoclaurine 3′-hydroxylase (TR\|O64901) |
| TC32376 | Cytochrome P450 (TR\|Q9LUC5); cytochrome P450 |
| | {Arabidopsis thaliana} |
| TC33268 | Cytochrome P450 81E1 (EC 1.14,-,-) |
| | (SP\|P93147\|C81E_GLYEC); CYTOCHROME P450 81E1 |
| | (EC 1.14,-,-) (ISOFLAVONE 2′-HYDROXYLASE) |
| | (P450 91A4) (CYP GE-3) [Licorice] |
| TC34135 | Cytochrome P450 (TR\|Q9AVQ2) |
| TC34228 | Cytochrome P450 71B2 (EC 1.14,-,-) |
| | (SP\|O65788\|C722_ARATH); CYTOCHROME P450 71B2 |
| | (EC 1.14,-,-) {Arabidopsis thaliana} |
| TC35157 | CYP83D1p (TR\|O48924) |
| TC36976 | Cytochrome P450 (TR\|Q9LUD2); cytochrome P450 |
| | {Arabidopsis thaliana} |
| TC37827 | Cytochrome P450 90A1 (EC 1.14,-,-) |
| | (SP\|Q42569\|C901_ARATH); CYTOCHROME P450 90A1 |
| | (EC 1.14,-,-) {Arabidopsis thaliana} |
| | (GP\|853719\|emb\|CAA60793) |
| TC40177 | CYP83D1p (TR\|O48924); CYP83D1p {Glycine max} |
| | (PIR\|T05940\|T05940) |
| TC40404 | Flavone synthase II (TR\|Q9SP27); flavone synthase II |
| | {Callistephus chinensis} |
| TC40527 | Putative cytochrome P450 (TR\|Q9XIQ1); cytochrome |
| | P450-like protein {Arabidopsis thaliana} |

TABLE 5
Medicago truncatula glycosyltransferase genes with similar
expression pattern to β-amyrin synthase based on cluster analysis

| | |
|---|---|
| TC29660 | Anthocyanidin-3-glucoside rhamnosyltransferase-like |
| | (TR\|Q9LTA3); anthocyanidin-3-glucoside |
| | rhamnosyltransferase-like {Arabidopsis thaliana} |
| T030007 | Glucosyltransferase-like protein (TR\|Q9LXV0); |
| | glucosyltransferase-like protein {Arabidopsis thaliana} |
| | (PIR\|T49903\|T49903) |
| TC30139 | F6F9.24 protein (TR\|Q9FXG9); Unknown Protein |
| | {Arabidopsis thaliana}(PIR\|A86330\|A86330) |
| TC31145 | UDP glucose:flavonoid 3-o-glucosyltransferase-like protein |
| | (TR\|Q9LFJ8); UDP-galactose:flavonol |
| | 3-O-galactosyltransferase {Petunia x hybrida} |
| TC31370 | Arbutin synthase (TR\|Q9AR73); arbutin synthase |
| | {Rauvolfia serpentina} |
| TC32537 | UDP-GLUCOSE GLUCOSYLTRANSFERASE |
| | (T16E15.3 PROTEIN) (TR\|Q9ZWJ3); Putative UDP-glucose |
| | glucosyltransferase |
| | {Arabidopsis thaliana} (PIR\|H86356\|H86356) |
| TC33774 | F9D12.19 protein (TR\|O81504), weak similarity to |
| | cyclodextrin glycosyltransferase (EC 2.4.1.19) (TR\|O30565) |
| TC35770 | Putative anthocyanidin-3-glucoside rhamnosyltransferase |
| | (TR\|Q9ZQ54); anthocyanidin-3-glucoside |
| | rhamnosyltransferase-like {Arabidopsis thaliana} |
| TC36241 | Anthocyanin 5-O-glucosyltransferase (TR\|Q9SBQ2); |
| | anthocyanin 5-O-glucosyltransferase {Petunia x hybrida} |
| TC36622 | Immediate-early salicylate-induced glucosyltransferase |
| | (TR\|P93365); betanidin-5-O-glucosyltransferase |
| | {Dorotheanthus bellidiformis} |
| TC36660 | Putative anthocyanidin-3-glucoside rhamnosyltransferase |
| | (TR\|Q9ZQ54) |
| TC39869 | MGDG synthase type A (TR\|Q9FZL4); |
| | MGDG synthase type A {Glycine max} |
| TC40162 | Putative flavonol 3-O-glucosyltransferase (TR\|O82383); |
| | putative flavonol 3-O-glucosyltransferase |
| | {Arabidopsis thaliana}(PIR\|F84699\|F84699) |
| TC41438 | UDP-GLUCOSE GLUCOSYLTRANSFERASE |
| | (T16E15.3 PROTEIN) (TR\|Q9ZWJ3); UDP-glucose |
| | glucosyltransferase {Arabidopsis thaliana} |
| | (GP\|9392679\|gb\|AAF87256.1\|AC068562_3\|AC068562) |

TABLE 6
List of the P450 EST TC and singleton numbers from the insect herbivory
library that appear to have enhanced transcripts in response to MeJA elicitation in
M. truncatula root cell suspension cultures, associated with increased expression of β-AS

| | | Full | | BLASTX (nucl. Vs prot.) | |
|---|---|---|---|---|---|
| TC # | Clones | length | Position | Results [First hit] | E value |
| TC32040 | NF055B10IN | + | B-9 | Sp: C120_SYNY3 Putative | 1e−15 |
| | NF089C08IN | − | C-12 | Cytochrome P450 120 (EC 1.14.—.—) | |
| | NF101H09IN | − | D-10 | | |
| | NF110B12IN | + | E-2 | Same as above | 1e−17 |
| TC32167 | NF010A04IN | + | A-2 | Sp: C933_SOYBN Cytochrome | |
| | | | | P450 71B2 (EC 1.14.—.—) | |
| TC32376 | NF056F04IN | + | B-12 | (GB# AB 023038) | 8e−57 |
| | NF101C03IN | − | D-7 | ARATH Cytochrome P450 | |
| TC34228 | NF049B11IN | + | B-7 | Sp: C722_ARARH Cytochrome | 2e−38 |
| | | | | P450 71B2 (EC 1.14.—.—) | |
| None | NF034B09IN | + | B-1 | Sp: C722_ARATH Cytochrome | 3e−38 |
| | | | | P450 71B2 (EC 1.14.—.—) | |
| None | NF037B11IN | + | B-2 | Sp: CP72_CATRO Cytochrome | 2e−43 |
| | | | | P450 72A1 (EC 1.14.14.1) | |
| None | NF115B10IN | + | E-4 | Sp: C771_SOLME Cytochrome | 6e−16 |
| | | | | P450 71B2 (EC 1.14.—.—) | |

TABLE 7
List of the glycosyltransferase EST TC and singleton numbers from the insect herbivory
library that appear to have enhanced transcripts in response to MeJA elicitation in M. truncatula
root cell suspension cultures, associated with increased expression of β-AS

| | | Full | | BLASTX (nucl. Vs prot.) | |
|---|---|---|---|---|---|
| TC # | Clones | length | Position | Results [First hit] | E value |
| TC35915 | NF014C07IN | + | A6 | sp: IAAG_MAIZE Indole-3-acetate | 3e−40 |
| | NF043A02IN | + | B12 | beta-glucosyltransferase (EC 2.4 . . . | |
| | NF058G07IN | + | C10 | | |
| | NF062C04IN | + | C11 | | |
| TC33217 | NF033G12IN | + | B7 | sp: IAAG_MAIZE Indole-3-acetate | 1e−24 |
| | | | | beta-glucosyltransferase (EC 2.4 . . . | |
| TC35768 | NF036D10IN | − | B9 | sp: UFOG_PETHY Flavonol 3-O- | 2e−24 |
| | NF114C03IN | − | F8 | glucosyltransferase (EC 2.4.1.91) | |
| | NF120A09IN | + | G2 | | |
| TC31370 | NF049A10IN | + | C3 | sp: IAAG_MAIZE Indole-3-acetate | 1e−30 |
| | | | | beta-glucosyltransferase (EC 2.4 . . . | |
| TC40085 | NF063F05IN | + | C12 | sp: LGT_CITUN Limonoid UDP- | 1e−19 |
| | | | | glucosyltransferase (EC 2.4.1.210) | |
| TC36660 | NF092G09IN | + | E3 | sp: UFOG_PETHY Flavonol 3-O- | 3e−06 |
| | | | | glucosyltransferase (EC 2.4.1.91) | |
| TC35664 | NF102C11IN | + | F1 | sp: YDF7_SCHPO. Putative | 2e−43 |
| | | | | glucosyltransferase C17C9.07 (EC | |
| | | | | 2.4.1.—). | |
| TC36622 | NF101F07IN | + | E11 | sp: UFOG_PETHY Flavonol 3-O- | 2e−09 |
| | | | | glucosyltransferase (EC 2.4.1.91) | |
| TC35770 | NF109C06IN | + | F8 | sp: UFOG_PETHY Flavonol 3-O- | 2e−17 |
| | | | | glucosyltransferase (EC 2.4.1.91) | |
| TC39629 | NF119G11IN | + | G1 | sp: UFO5_MANES Flavonol 3-O- | 3e−16 |
| | | | | glucosyltransferase 5 (EC 2.4.1.91) | |

### REFERENCES

The references listed below are incorporated herein by reference to the extent that they supplement, explain, provide a background for, or teach methodology, techniques, and/or compositions employed herein.

U.S. Pat. No. 3,817,837

U.S. Pat. No. 3,850,752

U.S. Pat. No. 3,939,350

U.S. Pat. No. 3,996,345

U.S. Pat. No. 4,275,149

U.S. Pat. No. 4,277,437

U.S. Pat. No. 4,282,287

U.S. Pat. No. 4,366,241

U.S. Pat. No. 4,535,060

U.S. Pat. No. 4,542,102

U.S. Pat. No. 4,683,195

U.S. Pat. No. 4,683,202

U.S. Pat. No. 4,800,159

U.S. Pat. No. 5,188,642

U.S. Pat. No. 5,252,743

U.S. Pat. No. 5,302,523

U.S. Pat. No. 5,322,783

U.S. Pat. No. 5,384,253

U.S. Pat. No. 5,384,253

U.S. Pat. No. 5,412,087

U.S. Pat. No. 5,445,934

U.S. Pat. No. 5,464,765

U.S. Pat. No. 5,508,184

U.S. Pat. No. 5,508,468

U.S. Pat. No. 5,538,877

U.S. Pat. No. 5,538,880

U.S. Pat. No. 5,538,880

U.S. Pat. No. 5,545,818

U.S. Pat. No. 5,550,318

U.S. Pat. No. 5,563,055

U.S. Pat. No. 5,591,616

U.S. Pat. No. 5,610,042

U.S. Pat. No. 5,633,448

U.S. Pat. No. 5,994,076

U.S. Pat. No. 6,077,673

U.S. Pat. No. 6,287,768

Abdullah et al., Biotechnology, 4:1087, 1986.

Abe and Prestwich, Chem. Rev., 93:2189-2206, 1993.

Akalezi et al., Process Biochem., 34:639-64, 1999.

Altschul et al., Nucleic Acids Research, 25:3389-3402, 1997.

Bates, Mol. Biotechnol., 2(2):135-145, 1994.

Battraw and Hall, Theor. App. Genet., 82(2):161-168, 1991.

Behboudi et al., Scand. J. Immunol.. 50:371-377, 1999.

Bell et al., Nucleic Acids Res., 29:114-117, 2001.

Bevan et al., Nucleic Acids Research, 11(2):369-385, 1983.

Bhattacharjee, An, Gupta, J. Plant Bioch. Biotech., 6(2):69-73. 1997.

Bouchez et al., EMBO Journal, 8(13):4197-4204, 1989.

Bower et al., Plant Journal, 2:409-416. 1992.

Buchanan-Wollaston et al., Plant Cell Reports, 11:627-631. 1992

Buising and Benbow, Mol. Gen. Genet., 243(1):71-81. 1994.

Callis et al., Genes Dev., 1:1183-1200, 1987.

Capaldi et al., Biochem. Biophys. Res. Comm., 74(2):425-433, 1977.

Casa et al., Proc. Natl. Acad. Sci. USA, 90(23):11212-11216, 1993.

Chandler et al., Plant Cell, 1:1175-1183, 1989.

Chapple, Annu. Rev. Plant Physiol. Plant Mol. Biol., 49:311-343, 1998.

Cheeke, Nutr. Rep. Int., 13:315-324, 1976.

Christou; et al., Proc. Nat'l Acad. Sci. USA, 84(12):3962-3966, 1987.

Chu et al., Scientia Sinica, 18:659-668, 1975.

Church and Gilbert, Proc. Natl. Acad. Sci. USA, 81:1991-1995, 1984.

Conkling et al., Plant Physiol., 93:1203-1211, 1990.

Corey et al., Biochem. Biophys. Res. Comm., 219:327-331, 1996.

Corey et al., Proc. Natl. Acad. Sci. USA, 90:11628-11632, 1993.

Corey et al., Proc. Natl. Acad. Sci. USA, 91:2211-2215, 1994.

DE 3642 829

De Block et al., EMBO J., 6(9):2513-2518, 1987.

De Block et al., Plant Physiol., 91:694-701, 1989.

Dellaporta et al., In: Chromosome Structure and Function: Impact of New Concepts, 18th Stadler Genetics Symposium, 11:263-282, 1988.

D'Halluin et al., Plant Cell, 4(12):1495-1505, 1992.

Dixon et al., Planta, 151:272-280, 1981.

Dixon, In Comprehensive Natural Products Chemistry, Vol. 1, Sankawa (ed.), Elsevier, Oxford, 773-823, 1999.

Ebert et al., 84:5745-5749, Proc. Nat'l. Acad. Sci. USA, 1987.

Eisen et al., Proc. Natl. Acad. Sci. USA, 95:14863-14868, 1998.

Ellis et al., EMBO Journal, 6(11):3203-3208, 1987.

EPA 154,204

Fraley et al., Bio/Technology, 3:629-635, 1985.

Fromm et al., Nature, 319:791-793, 1986.

Fulcheri et al., J. Agric. Food Chem., 46:2055-2061, 1998.

Gallie et al., The Plant Cell, 1:301-311, 1989.

Gelvin et al., In: Plant Molecular Biology Manual, 1990.

Ghosh-Biswas et al., J. Biotechnol., 32(1):1-10, 1994.

Gundlach et al., Proc. Natl. Acad. Sci. USA, 89:2389-2393, 1992.

Hagio et al., Plant Cell Rep., 10(5):260-264, 1991.

Hamilton et al., Proc. Natl. Acad. Sci. USA, 93(18):9975-9979, 1996.

Haralampidis et al., Proc. Natl. Acad. Sci. USA, 98:13431-13436, 2001.

Haridas et al., Proc. Natl. Acad. Sci. USA, 98:5821-5826, 2001.

Haseloff et al., Proc. Natl. Acad. Sci. USA, 94(6):2122-2127, 1997.

Hayashi et al., Biol. Pharm. Bull., 23:231-234, 2000.

He et al., Plant Cell Reports, 14 (2-3):192-196, 1994.

Hensgens et al., Plant Mol. Biol., 22(6):1101-1127, 1993.

Herold and Henry, Biotech. Lett., 23:335-337, 2001.

Hiei et al., Plant. Mol. Biol., 35(1-2):205-218, 1997.

Hinchee et al., Bio/technol., 6:915-922, 1988.

Hou and Lin, Plant Physiology, 111:166, 1996.

Hudspeth and Grula, Plant Mol. Biol., 12:579-589, 1989.

Huhman and Sumner, Phytochemistry, 59:347-360, 2002.

Husselstein-Muller et al., Plant Mol Biol, 45:75-92, 2001.

Ikuta et al., Bio/technol., 8:241-242, 1990.

Immobilized Biochemicals and Affinity Chromatography, Adv. Exp. Med. Biol., 42 Dunlap (Ed.), Plenum Press, NF, 1974

Ishidia et al., Nat. Biotechnol., 14(6):745-750, 1996.

Jandrositz et al., Gene, 107:155-160, 1991.

Jones and Elliott, Crop Sci., 9:688-691, 1969.

Kaeppler et al., Plant Cell Reports 9:415-418, 1990.

Kaeppler, Somers, Rines, Cockburn, Theor. Appl. Genet., 84(5-6):560-566, 1992.

Katz et al., J. Gen. Microbiol., 129:2703-2714, 1983.

Klee, Yanofsky, Nester, Bio-Technology, 3(7):637-642, 1985.

Knittel, Gruber; Hahne; Lenee, Plant Cell Reports, 14(2-3):81-86, 1994.

Kribii et al., Eur. J. Biochem., 249:61-69, 1997.

Kushiro et al., Eur. J Biochem., 256:238-244, 1998.

Kushiro et al., Tetrahedron Lett., 41:7705-7710, 2000.

Laden et al., Arch. Biochem. Biophys,. 374:381-388, 2000.

Landl et al., Yeast, 12:609-613, 1996.

Lawton et al., Plant Mol. Biol. 9:315-324, 1987.

Lazzeri, Methods Mol. Biol., 49:95-106, 1995.

Leber et al., Mol. Biol. Cell. 9:375-386, 1998.

Lee et al., Arch. Biochem. Biophys., 381:43-52, 2000.

Lee; Suh; Lee, Korean J. Genet., 11(2):65-72, 1989.

Lorz et al., Mol Gen Genet, 199:178-182, 1985.

Marciani et al., Vaccine, 18:3141-3151, 2000.

Marcotte et al., Nature, 335:454, 1988.

Massiot et al., J Chem. Soc. Perkin Trans., 3071-3079, 1988.

McCabe, Martinell, Bio-Technology, 11(5):596-598, 1993.

McCormac et al., Euphytica, 99 (1):17-25, 1998.

Memelink et al., Trends Plant Sci,. 6:212-219, 2001.

Morita et al., Biol. Pharm. Bull., 20:770-775, 1997.

Morita et al., Eur. J. Biochem., 267:3453-3460, 2000.

Murakami et al., Mol. Gen. Genet., 205:42-50, 1986.

Murashige and Skoog, Physiol. Plant., 15:473-497, 1962.

Nagata et al., Camellia japonica. Agric., Biol. Chem., 49:1181-1186, 1985.

Nagatani et al., Biotech. Tech., 11(7):471-473, 1997.

Nakamura et al., In: Handbook of Experimental Immunology (4^{th} Ed.), Weir et al., (Eds). 1:27, Blackwell Scientific Publ., Oxford, 1987.

Nakashima et al., Proc. Natl. Acad. Sci. USA, 92:2328-2332, 1995.

Odell et al., Nature, 313:810-812, 1985.

Ogawa et al., Sci. Rep., 13:42-48, 1973.

Oleszek and Jurzysta, J. Chromatog., 519:109-116, 1990.

Oleszek et al., J. Agric. Food Chem., 40:191-196, 1992.

Oleszek et al., J. Agric. Food. Chem., 38:1810-1817, 1990.

Oleszek et la., J. Agric. Food Chem., 47:3685-3687, 1999.

Oleszek, In Saponins Used in Food and Agriculture, Waller and Yamasaki (Eds.), Plenum Press, NY, 155-170, 1997.

Oleszek, J. Sci. Food Agric., 44:43-49, 1988.

Omirulleh et al., Plant Mol. Biol., 21(3):415-428, 1993.

Ono and Bloch, J. Biol. Chem., 250:1571-1579, 1975.

Osbourn, Trends Plant Sci., 1:4-9, 1996.

Ow et al., Science, 234:856-859, 1986.

Pandit J. Biol. Chem., 275:30610-30617, 2000.

Papadopoulou et al., Proc. Natl. Acad. Sci. USA, 96:12923-12928, 1999.

Park et al., Planta Med., 67:118-121, 2001.

PCT Appl. WO 9217598

PCT Appl. WO 94/09699

PCT Appl. WO 95/06128

PCT Appl. WO 95/06128

PCT Appl. WO 97/04103

PCT Appl. WO 97/04103

PCT Appl. WO 97/41228

PCT Pub. No. 90/07582

PCT Pub. No. 91/00868

PCT Pub. No. 91/07087

Pedersen et al., Crop Sci., 16, 193-199, 1976.

Potrykus et al., Mol. Gen. Genet., 199:183-188, 1985.

Prasher et al, Biochem. Biophys. Res. Commun., 126(3):1259-1268, 1985.

Quackenbush et al., Nucleic Acids Res.,. 28:141-145, 2000.

Reichel et al., Proc. Natl. Acad. Sci. USA, 93 (12):5888-5893. 1996.

Rhodes et al., Methods Mol. Biol., 55:121-131, 1995.

Ritala et al., Plant Mol. Biol., 24(2):317-325, 1994.

Rogers et al., Methods Enzymol., 153:253-277, 1987.

Sakakibara et al., J Biol. Chem., 270:17-20, 1995.

Sambrook et al.,, Molecular Cloning: A Laboratory Manual. (Cold Spring Harbor, N.Y.: Cold Spring Harbor Laboratory Press, 2001.

Schäfer et al., Plant Mol. Biol, 39:721-728, 1999.

Segura et al., Org. Lett., 2:2257-2259, 2000.

Sheen et al., Plant Journal, 8(5):777-784, 1995.

Shibata et al., Proc. Natl. Acad. Sci. USA, 98:2244-2249, 2001.

Singsit et al., Transgenic Res., 6(2):169-176, 1997.

Spencer et al., Plant Molecular Biology, 18:201-210, 1992.

Stalker et al., Science, 242:419-422, 1988.

Sullivan et al., Mol. Gen. Genet., 215(3):431-440, 1989.

Sutcliffe, Proc. Natl. Acad. Sci. USA, 75:3737-3741, 1978.

Tamayo, Proc. Natl. Acad. Sci. USA, 96:2907-2912, 1999.

Tava and Odorati, In: Saponins Used in Food and Agriculture, Waller and Yamasaki (Eds.,) Plenum Press, NY, 97-109, 1997.

Tava et al., Phytochem. Anal., 4:269-274, 1993.

Thillet et al., J. Biol. Chem., 263:12500-12508, 1988.

Thomas et al., Plant Sci., 69:189-198, 1990.

Thompson et al., Euphytica, 85(1-3):75-80, 1995.

Thompson et al., EMBO J., 6(9):2519-2523, 1987.

Tian et al., Plant Cell Rep., 16:267-271, 1997.

Tingay et al., Plant Journal, 1(6):1369-1376, 1997.

Tomes et al., Plant. Mol. Biol., 14(2):261-268, 1990.

Torbet et al., Crop Science, 38(1):226-231, 1998.

Torbet et al., Plant Cell Reports, 14(10):635-640, 1995.

Toriyama et al., Theor. Appl. Genet., 73:16, 1986.

Tsukada et al., Plant Cell Physiol., 30(4)599-604, 1989.

Twell et al., Plant Physiol, 91:1270-1274, 1989.

Uchimiya et al., Mol. Gen. Genet., 204:204, 1986.

Van Eck et al., Plant Cell Reports, 14(5):299-304, 1995.

Vasil et al., Plant Physiol., 91:1575-1579, 1989.

Vogt and Jones, Trends in Plant Science, 5:380-386, 2000.

Walker et al., Proc. Natl. Acad. Sci. USA, 84:6624-6628, 1987.

Waller et al., Bot. Bull. Acad. Sin, 34:1-11, 1993.

Wang et al., Molecular and Cellular Biology, 12(8):3399-3406, 1992.

Yamada et al., Plant Cell Rep., 4:85, 1986.

Yang and Russell, Proc. Natl. Acad. Sci. USA, 87:4144-4148, 1990.

Yoon et al., Biotechnol.Lett. 22, 13:1071-75, 2000.

Zheng and Edwards, J. Gen. Virol., 71:1865-1868, 1990.

Zhou et al., Plant Cell Reports, 12(11).612-616, 1993.

Zukowsky et al., Proc. Natl. Acad. Sci. USA, 80:1101-1105, 1983.

What is claimed is:

1. A method of identifying a triterpene biosynthesis coding sequence comprising:
(a) obtaining a cell from a target legume species;
(b) contacting said cell with methyl jasmonate; and
(c) identifying a coding sequence which is specifically upregulated in the cell following the contacting with methyl jasmonate to identify a triterpene biosynthesis gene.

2. The method of claim 1, further comprising screening a polypeptide encoded by the coding sequence for the ability to catalyze a step in triterpene biosynthesis.

3. The method of claim 1, wherein the target legume is selected from the group consisting of soybean, alfalfa, Medicago truncatula, peanuts, beans, peas, lentils, Lotus japonicus, chickpea, cowpea, lupin, vetch, Sophora species, Acacia species, licorice and clover.

4. The method of claim 3, wherein the target legume is Medicago truncatula.

5. The method of claim 1, wherein the cell is grown in a tissue culture.

6. The method of claim 5, wherein the tissue culture is a cell suspension culture.

7. The method of claim 1, wherein the cell is obtained from a plant treated with said methyl jasmonate.

8. The method of claim 1, wherein the step of obtaining a cell is further defined as comprising obtaining a population of cells from the target legume.

9. The method of claim 8, comprising preparing a tissue culture from the cell.

10. The method of claim 1, wherein the step of identifying a coding sequence is further defined as comprising identifying a plurality of coding sequences specifically upregulated in said cell relative to the corresponding coding sequences in one or more other cells which have not been contacted with methyl jasmonate.

11. The method of claim 1, wherein the step of identifying a coding sequence comprises obtaining an RNA transcribed by the coding sequence and/or a cDNA derived therefrom.

12. The method of claim 11, further comprising the steps of:
(a) labeling said RNA and/or cDNA; and
(b) hybridizing the labeled RNA or cDNA to an array comprising a plurality of coding sequences from the target legume.

13. The method of claim 10, further comprising preparing an array comprising the RNA transcripts or cDNAs thereof arranged on a support material.

14. The method of claim 1, wherein identifying a coding sequence further comprises selecting a coding sequence having homology to a cytochrome P450.

15. The method of claim 13, wherein identifying a coding sequence further comprises selecting a coding sequence having homology to glycosyltransferase.

16. The method of claim 1, wherein identifying a coding sequence further comprises selecting a coding sequence having homology to a squalene synthase.

17. The method of claim 1, wherein identifying a coding sequence further comprises selecting a coding sequence having homology to a squalene epoxidase.

18. The method of claim 1, wherein identifying a coding sequence further comprises selecting a coding sequence having homology to β-amyrin synthase.

19. The method of claim 1, wherein identifying a coding sequence comprises use of subtractive hybridization.

20. The method of claim 1, wherein identifying a coding sequence comprises use of nucleic acid sequencing.

21. The method of claim 1, wherein identifying a coding sequence comprises use of RT-PCR.

22. The method of claim 1, wherein identifying a coding sequence comprises use of differential display.

23. The method of claim 1, wherein identifying a coding sequence comprises use of an array.

24. The method of claim 1, wherein screening comprises transforming a host cell with the coding sequence and determining the ability of the host cell to catalyze a step in triterpene biosynthesis.

25. The method of claim 24, further comprising contacting the host cell with a substrate of said step in triterpene biosynthesis.

26. The method of claim 25, wherein the substrate is selected from the group consisting of farnesyl diphosphate, squalene, oxidosqualene and β-amyrin.

27. The method of claim 25, wherein the substrate is selected from the group consisting of bayogenin, hederagenin, medicagenic acid, soyasapogenol B and soyasapogenol E.

28. The method of claim 24, wherein the host cell is a yeast cell.

29. The method of claim 24, wherein the host cell is a plant cell.

30. The method of claim 29, further comprising regenerating a plant from the plant cell.

31. The method of claim 24, wherein the host cell is a bacterial cell.


SEQUENCE LISTING (31 sequences)

SEQ ID NO: 1 - DNA, 1210 bp, Medicago sativa
tatagggcgtcgactcgatccactccctgaggtaccggtcttagaatcaacatgaaatgcatggacttaacgtgttattggtgttttctactgattggtgttaaatcgaacaacacctaacctaaacctaactatgaaattcaacatgtgaactattttaaacacgttacaattttagagattatttatcatttcttcatatgtaaaaactaattagaccaatcctaacaatttcatcatgcatggaattatatggcttagtggaataatggatgaccgactaggaactactacaggaaggatgggtagtcaatgcaacgtgagagtgggagttggatcagtcgtccaatactcatggggcgcaaaatagtcgacagactaatttgtttgttttgagaattgcaaaataccaatacaatgtttgaaaaattaataaaacataatttgcagtttcaaaacattgcattttggaccctgtgagcttaactcagttgataagaagaatgtataatatatgcaagatccggggttcttataaaaaaggacgtatatcaacgaaaaataatcataataattctacgttcgatcaagaagtaagtaaaatttgatcaataattattccatctaaaaatcaaatttagattctcctaaacaattttttttagtggatctcattaactaacttagtctatcatccagctatttaataaaaaaaattgatttttctccttctacttatcttttaatgtgagtgtgactaaatagtttaaatttaaacttgtgaccacacgactcttctaataatagggttgtttactctttagttgtttttcaacaagcaagcaaggaaaggagcatttgatatgagtcaaaagagcgcgggactttgagtcttatccaacacgtacacggcgcacattagtacaaatcctttctcttgttttcaaagagtagacagctaataacatgcgtccgtggccgtctcgcatctctcatgcattataaaatgcgtcctactttccctcactttctgttacatccaaaacccttcatttttcctctgccaacttggcagagagagagagagagagagagagagagagagagagagagagagagagaagaatctcaaacgcaaaatattacacctaattctccgtcgtcatcaccactgtcaccatcgccggcgagattcctccgatatggatct

SEQ ID NO: 2 - DNA, 1581 bp, Medicago truncatula
  CDS (1)..(1581)
atggatctatacaatatcggttggattttaagctctgttttgagtctatttgcgttatacaatttgattttcgccgggaagaagaattatgatgtgaatgagaaggtaaatcagcgtgaggatagcgtgacgagtactgatgccggtgaaattaaatcggacaaacttaacggtgatgctgatgttatcattgttggagctggtattgctggtgctgctttggctcatacacttgggaaggatggacgtcgagtgcatattattgaaagagatttgagtgagcctgacagaattgttggagagttgctacaacccggtggctatctcaaattagttgaactcgggcttcaagattgtgtggacaatattgatgcacagcgagtgtttggttatgctctttttaaggacgggaaacatactcgtctctcttatcccttggaaaagtttcactcagatgtctctggcagaagctttcacaatgggcgttttattcagaggatgcgagagaaagctgcctcacttcccaatgtaaatatggagcaaggaacagtcatttccctacttgaagagaaggggacaatcaaaggtgtgcaatacaagaataaagatggtcaggcattgacagcatatgctcctcttaccattgtttgtgatggctgtttctcaaacttgcgtcgttctctttgcaaccctaaggtagataatccctcttgttttgttggcttaattttagagaactgtgaacttccatgtgctaatcacggccatgtcatacttggagatccttcgccaattcttttctatcctataagcagtacagagattcgttgtctggttgatgtacctggaacgaaggttccttctatttcaaacggtgatatgacaaagtatctaaagacgacagttgctccacaggtcccccctgagctttatgatgcattcatagccgcagtggacaaaggcaacataaggacaatgccaaacagaagtatgccagcagatcctcgtcctactcctggagccgtactgatgggagatgcattcaacatgcgtcatccactaacagggggcggaatgaccgtagcattgtctgacattgtggtgttgagaaatcttctcaagcctatgcgtgacctgaacgatgcacctacactttgcaaatacctcgaatccttttataccttgcggaagcctgtggcatccaccataaatacattggcaggagccctttacaaggttttcagtgcatcccccgatgaagcaaggaaggaaatgcgccaagcttgttttgattatctcagccttggaggcttattctcagaaggaccgatctctttactttcaggattaaaccctcggcccttaagcttggttctccatttctttgctgtcgcggtatttggtgttggccgtttactattaccatttccttcacctaagcgggtgtggattggagctcgattactctctggtgcatctggaatcattttacccataattaaggccgaaggaattcggcagatgtttttccctgccactgttccagcttattacagagctcccccggtaaatgcattttga

SEQ ID NO: 3 - protein, 526 aa, Medicago truncatula
MDLYNIGWILSSVLSLFALYNLIFAGKKNYDVNEKVNQREDSVTSTDAGEIKSDKLNGDADVIIVGAGIAGAALAHTLGKDGRRVHIIERDLSEPDRIVGELLQPGGYLKLVELGLQDCVDNIDAQRVFGYALFKDGKHTRLSYPLEKFHSDVSGRSFHNGRFIQRMREKAASLPNVNMEQGTVISLLEEKGTIKGVQYKNKDGQALTAYAPLTIVCDGCFSNLRRSLCNPKVDNPSCFVGLILENCELPCANHGHVILGDPSPILFYPISSTEIRCLVDVPGTKVPSISNGDMTKYLKTTVAPQVPPELYDAFIAAVDKGNIRTMPNRSMPADPRPTPGAVLMGDAFNMRHPLTGGGMTVALSDIVVLRNLLKPMRDLNDAPTLCKYLESFYTLRKPVASTINTLAGALYKVFSASPDEARKEMRQACFDYLSLGGLFSEGPISLLSGLNPRPLSLVLHFFAVAVFGVGRLLLPFPSPKRVWIGARLLSGASGIILPIIKAEGIRQMFFPATVPAYYRAPPVNAF

SEQ ID NO: 4 - DNA, 1242 bp, Medicago truncatula
  CDS (1)..(1242)
atgggaagtataaaagcgattttgaagaatccagatgatttctttccattacttaagctgaaaatcgcggccagaaacgccgagaagcagatcccaccggaaccgcattggggattctgttactctatgcttcataaggtttctagaagcttcggtcttgttattcagcagcttggtcctgagcttcgtgatgctgtttgcatattctatttggttcttcgcgctcttgataccgttgaggatgatacaagcatagaaacagatgtcaaggttcccatactaatagattttcatcgtcacatttatgataatgattggcactttgggtgtggcacgaaggagtacaaagttctaatggaccagtttcatcatgtttcaaaggcttttctggaacttggaaagaactatcaggatgcaatcgaggacattacgaaaagaatgggtgctggaatggcgaaattcatttgcaaggaggtagaaacagttgatgactacgatgaatattgtcactatgtggctggacttgttgggctgggtttatcaaagcttttctatgcctctggtaaagaagatctggctacagacaaactttcaaattcaatgggtttgtttcttcagaaaaccaacattattcgagattatctggaagacatcaatgagataccaaagtcacgcatgttttggccacggcagatctggagtaaatatgttagcaaacttgaggacttgaaatatgaggaaaactccgttaaggctgtgcaatgcttaaatgacatggtcactaatgctttgctgcatgctgacgattgcttacaatacatgtctgcattacgagactcctctaattttcgcttttgtgctattcctcaggtaatggcaattggaacacttgcaatgtgctacaacaacattggtgtcttcagaggtgtagttaaaatgaggcgaggtctaactgccaaagtgattgaccgaaccaagactatggctgatgtctatggtgctttctttgattttgcttccgtgttggagtccaaggttgacaaaaatgatccaaatgcaacaaaaacatcgagcaggctggaagctatacagaaaacttgcagagaatctggtctcctaaccaaaaggaaatcttacgttttgaggaatgagagcggatatggctctaccatgattctcttactggtcatcttgttttccatcatttttgcttatctctctgccaatcgtcacaataactaa

SEQ ID NO: 5 - protein, 413 aa, Medicago truncatula
MGSIKAILKNPDDFFPLLKLKIAARNAEKQIPPEPHWGFCYSMLHKVSRSFGLVIQQLGPELRDAVCIFYLVLRALDTVEDDTSIETDVKVPILIDFHRHIYDNDWHFGCGTKEYKVLMDQFHHVSKAFLELGKNYQDAIEDITKRMGAGMAKFICKEVETVDDYDEYCHYVAGLVGLGLSKLFYASGKEDLATDKLSNSMGLFLQKTNIIRDYLEDINEIPKSRMFWPRQIWSKYVSKLEDLKYEENSVKAVQCLNDMVTNALLHADDCLQYMSALRDSSNFRFCAIPQVMAIGTLAMCYNNIGVFRGVVKMRRGLTAKVIDRTKTMADVYGAFFDFASVLESKVDKNDPNATKTSSRLEAIQKTCRESGLLTKRKSYVLRNESGYGSTMILLLVILFSIIFAYLSANRHNN

SEQ ID NO: 6 - DNA, 2067 bp, Medicago truncatula
  CDS (1)..(2067)
atgcaaacaatagatggagtgaagatagaagatggagaagagataacatatgagaaagcaacgacaacgttgagaaggggcacacaccatctagcagcattgcaaaccagtgatggccattggcctgctcaaattgcaggtcctctatttttcatgcctcccttggttttctgtgtctacattactggacatcttgattccgtcttcccacgagaacatcgcaaagagattcttcgttacatttactgtcaccaaaatgaagatggaggatgggggctacacattgagggtcacagcaccatgttttgtactgcacttaactatatatgtatgcgaattctcggagaaggacctgatggcggtcaagacaatgcttgtgctagagccagaaactggattcgggcacacggtggtgtcacatatataccttcatggggaaaaacttggctttcgatacttggtctctttgattggttgggaagcaacccaatgccccctgagttttggatccttccttcatttcttcctatgcatccagctaaaatgtggtgttattgtcgattggtatacatgcctatgtcttacttgtacgggaagagatttgtgggtccgatcacaccactcatcttacagttgagagaagaactccatactcagccttatgaaaaaattaactggacgaaatcacgtcacctatgtgcaaaggaagatatttactatccccatcctttgatacaagatctgatatgggatagcttatacatatttaccgagccgcttctcactcgctggcctttcaacaagctggtcagaaaaagagcccttgaagttacaatgaagcatatccactacgaggatgagaacagtcgatacctaaccattgggtgtgtggaaaaggtattatgtatgcttgcttgttgggtggaagatccaaatggagatgcttacaagaagcatcttgcaagggtccaagattacttgtggatgtcagaagatggaatgaccatgcagagttttggtagccaagaatgggatgctggttttgccgttcaagctttgcttgccgctaacctaaatgatgaaatcgaacctgcacttgccaaaggacatgatttcattaagaaatctcaggttacagagaacccttctggagattttaagagtatgcatcgtcatatttctaaaggctcatggaccttctccgatcaagaccatggatggcaagtttctgattgcaccgctgaaggtttgaagtgttgtctacttttatcaatgttgcctccagagattgtgggggaaaagatggaaccagaaaggttatatgattcggtcaatgtcttgttgtcgcttcagagtaaaaagggtggtttggcagcatgggagcccgcaggagctcaagagtggttagaactactcaatcccactgagttttttgcggacattgttgttgagcatgaatatgttgagtgcactggatcagcaattcaagctttagttttgttcaagaagctatatccagggcataggaagaaagagatagagaatttcatctccgaggcagttcgattcattgaagatatacaaacagccgatggttcatggtatggaaactggggagtttgcttcacttatggttcttggtttgctcttggtggtttagcagctgctggcaagacttataccaattgcgctgctattcgcaaagctgttaaatttcttctcacaacacagagagaggatggtgggtggggggagagctatctttcaagcccaaaaaagatatatgtacctctcgaaggaagccgatccaatgttgtacatactgcatgggctcttatgggtttaattcatgccggccaggcagagagagaccctactcctctccatcgtgctgcaaaattgctcatcaattcccagttggaagaaggcgattggccccaacaggaaatcacaggagtattcatgaaaaattgtatgttgcattacccaatgtatagagatatttaccccttgtgggctctagccgagtatcgtagacgggttccattgccttccactgcagtttaa

SEQ ID NO: 7 - protein, 688 aa, Medicago truncatula
MQTIDGVKIEDGEEITYEKATTTLRRGTHHLAALQTSDGHWPAQIAGPLFFMPPLVFCVYITGHLDSVFPREHRKEILRYIYCHQNEDGGWGLHIEGHSTMFCTALNYICMRILGEGPDGGQDNACARARNWIRAHGGVTYIPSWGKTWLSILGLFDWLGSNPMPPEFWILPSFLPMHPAKMWCYCRLVYMPMSYLYGKRFVGPITPLILQLREELHTQPYEKINWTKSRHLCAKEDIYYPHPLIQDLIWDSLYIFTEPLLTRWPFNKLVRKRALEVTMKHIHYEDENSRYLTIGCVEKVLCMLACWVEDPNGDAYKKHLARVQDYLWMSEDGMTMQSFGSQEWDAGFAVQALLAANLNDEIEPALAKGHDFIKKSQVTENPSGDFKSMHRHISKGSWTFSDQDHGWQVSDCTAEGLKCCLLLSMLPPEIVGEKMEPERLYDSVNVLLSLQSKKGGLAAWEPAGAQEWLELLNPTEFFADIVVEHEYVECTGSAIQALVLFKKLYPGHRKKEIENFISEAVRFIEDIQTADGSWYGNWGVCFTYGSWFALGGLAAAGKTYTNCAAIRKAVKFLLTTQREDGGWGESYLSSPKKIYVPLEGSRSNVVHTAWALMGLIHAGQAERDPTPLHRAAKLLINSQLEEGDWPQQEITGVFMKNCMLHYPMYRDIYPLWALAEYRRRVPLPSTAV

SEQ ID NO: 8 - DNA, 38 bp, Artificial Sequence
  Description of Artificial Sequence  Synthetic Primer
ccatgccatgggaagtataaaagcgattttgaagaatc

SEQ ID NO: 9 - DNA, 35 bp, Artificial Sequence
  Description of Artificial Sequence  Synthetic Primer
cgggatccttagttattgtgacgattggcagagag

SEQ ID NO: 10 - DNA, 36 bp, Artificial Sequence
  Description of Artificial Sequence  Synthetic Primer
cgcggatccatgatagacccctacggtttcgggtgg

SEQ ID NO: 11 - DNA, 35 bp, Artificial Sequence
  Description of Artificial Sequence  Synthetic Primer
ccgctcgagttatgcatctggaggagctctataat

SEQ ID NO: 12 - DNA, 37 bp, Artificial Sequence
  Description of Artificial Sequence  Synthetic Primer
cgcggatccatgtcttttaatcccaacggcgatgttg

SEQ ID NO: 13 - DNA, 39 bp, Artificial Sequence
  Description of Artificial Sequence  Synthetic Primer
cgcggatccatggatctatacaatatcggttggaattta

SEQ ID NO: 14 - DNA, 35 bp, Artificial Sequence
  Description of Artificial Sequence  Synthetic Primer
ccgctcgagtcaaaatgcatttaccgggggagctc

SEQ ID NO: 15 - DNA, 37 bp, Artificial Sequence
  Description of Artificial Sequence  Synthetic Primer
cgcggatccatgtcggacaaacttaacggtgatgctg

SEQ ID NO: 16 - DNA, 38 bp, Artificial Sequence
  Description of Artificial Sequence  Synthetic Primer
cgggatccatgtctgctgttaacgttgcacctgaattg

SEQ ID NO: 17 - DNA, 40 bp, Artificial Sequence
  Description of Artificial Sequence  Synthetic Primer
ccgctcgagttaaccaatcaactcaccaaacaaaaatggg

SEQ ID NO: 18 - DNA, 2581 bp, Artificial Sequence
  Description of Artificial Sequence  Synthetic Primer
ggctctttctctctgctcccatctacttcacacctaccacacaagacgagagagactctttcttcaattcaattcatttcatttcatttcattttttttaaccatgcaagattcaagctcaatgaaattttctccacttgatctaatgtcagcactaatcaaaggcaaaatcgatccatccaatggaaccgttccagcttcactcatacttgagaaccgcgaattcgttatgatcttaacaacttcaatagctgttctcatcggttgcgtcgtcgttttaatttggcgtagatccaattctcaaaaaccaaaaccaattgaagttcctaaacgcgttatcgagaaacttcctgaacttgaaatcgatgacggtaccaaaaaagttaccgttttctttggcactcaaaccggtaccgccgaaggttttgccaaggcgatagcggaagaggcaaaagcgcgttatgagaaggccaagtttaaagtagttgatatggatgattatgctgctgatgacgatgaatacgaggagaaattaaaaaaggagacaatggctcttttcttcttagctacatatggtgatggtgagccaactgataatgccgcgagattttataaatggttcgaggaatttgaaggggaagaagattcgtttaagaatcttcagtatggtgtgtttggacttgggaacagacagtatgagcattttaataaggtggctaaaatagttgatgataagcttcttgagaaaggtgggaatcgtcttgtccccgtgggtcttggagatgatgatcagtgtatagaagatgattttactgcatggaaagaagaactatggccagcgttggatcaattgctaagagatgaggatgatgcaactgtggctacaccttatactgcttctgttttggagtaccgggttgttattcgtgatcaattggatgcaactgtggacgaaaagaagcagcttaatggaaatggccatgctgttgtggacgctcatcatccagtcagggctaatgtggctgtgcgaaaggagcttcatactcctgcatcagatcgttcttgcactcatttagaatttgacatttcaggcaccggagttgtatatgaaacaggggaccatgttggtgtttactgtgagaatttatccgacactgtggaagaggcagaaaggatactaggtttgtccccggacacctatttctccgtccataccgatgacgaagatgggaaacctcttggtggaagctccttgcctcctcctttcccaccctgtactttaagaacagcgcttgctaaatacgcagatgttttgagttcacctaaaaagtctgccttgcttgccttagctgctcatgcatctgatccatctgaagcggatcgactaagacatcttgcctcacctgctggaaaggatgagtatgcagagtgggtgattgcctctcaaagaagtctccttgaggttatggctgaattttcatcagccaaacctccaattggtgtcttttttgcatcagttgctcctcgcctacagccaagatattattcaatttcatcatctccaagagtggcaccatccagaattcatgttacctgcgcgttagtgcatgataaaatgcccactggacggattcatcaaggagtgtgttcaacttggatgaagaattctgcaccattggagaaaagtcaggactgtagttgggctcctatctttgttaggcagtccaatttcagactccctgctgataataaagtgcctataattatgataggtcctgscactgggttggctccttycagaggtttcttgcaggaaagattagctttgaaagaagaaggagctgagctaggcccctctgttttattctttggttgcaggaaccgtcaagtggactatatctacgaagatgaattgaaccatttcgttcatggtggcgcactttctgagctcattgttgccttctcacgagaggggcctactaaggaatatgtccaacataaaatgatagagaaggcttcagatatttggaacatgatatctcagggagcttacatttatgtgtgtggtgatgccaagggtatggctaaggatgtacaccgcactctacatacaattttgcaagaacagggctctttggacaattccaagactgagagcatggttaagaacctacaaatgactggcagatatttgcgtgatgtatggtaatgatgaaccgggcttatgataaaacgccagtaaagactagtaatggattgaagataagatattggaagggacttatttattccctttcagttatgccctcaaaagagggagaaggggttacttctgcgtgttacatcacggcaacaattcctacgattctttgtcactattttcaaatggattcttttttgttcgtgtaacttatactttgtttatacatagattatgtattttagctttcttatattgtatgaacctataaggagggttttgagatcagacaattccttgtattgtaaacctctaataaaattgtttgcagaaaaatgagaattatttctgctagtattatatcaaaaaaaaaaaaaaaaa

SEQ ID NO: 19 - DNA, 1721 bp, Artificial Sequence
  Description of Artificial Sequence  Synthetic Primer
ggttttttttctctatattcaatatcaacctgtttccaaccttctctctgttcaaacacacaattcatcacaatggctctgtttctcacaatacccctttcattcatagccattttcctcttttacacactcttccaaagactgagattcaagcttccacccggtccacgaccgtggccggtggttggaaacctctacgacataaaacctgtccggttcaggtgttttgccgaatgggcccaatcctatgggcccattatatcggtttggtttggttcgactctgaacgtgatcgtttcaaattcaaagttggctaaagaagttttaaaggagaatgatcagcagttggctgaccggcacagaagtcggtcagcggcaaagtttagtagagatgggcaggatttaatttgggctgattatggaccccattatgtgaaggttaggaaggtttgtacgttagagcttttttcacctaagagaattgaagctttgaggcctattagagaagatgaggttactgctatggttgaatccattttcaatgattctaccaattctgaaaatttggggaaaggtatactgatgaggaagtatataggggcagttgcattcaacaacatcaccaggttggcatttgggaaaagatttgtgaactcagaaggtgtaatggatgagcaaggagtagaattcaaggctatagtggcaaatggattaaagctaggagcatctctagctatggcagagcacatcccttggttgcgctggatgtttccacttgaagaggaggcttttgctaagcacggtgctcgtagggaccggctcaccagagccatcatggaagagcatacgcaggcacgtcagaaatccggtggtgccaaacaacattttgtagatgcacttctcactttgcaagagaaatatgaccttagtgaagacaccatcattggtctcctttgggacatgattacagctgggatggacacaactgcaatatcagttgagtgggccatggcagagctgataaagaatccaagagtgcaacagaaggcacaagaggagctagacaaggtcattggttttgaaagagtcatgactgaaactgacttctcaagcctcccttatttacaatgtgtagccaaggaggctctaaggctgcaccccccaacaccattaatgctcccacatcgtgctaacaccaatgtcaaaatcgggggctatgatattcccaaagggtcaaatgtccacgtaaatgtatgggctgttgcgcgtgatccagctgtttggaaagacgcaacagagtttagacccgagaggtttcttgaggaggatgtagacatgaagggtcatgactttaggctacttccatttggagcaggtcgtcgagtatgtccaggggcacaacttgggatcaatatggtgacatccatgttgggtcatctattgcaccatttctgctgggcaccacccgagggagtgaacccagcggagattgacatggcagagaaccctggaatggttacatacatgaggactccattacaggttgtggcctctcctaggcttccgtcggagttgtacaaacgtgtgacagctgatatctaatctttttctcatactgcaatgttgctgtttttcaaaatgttgagtcaattttcttatgggatttatttctaccattgtgtctatgtaactataattggaataaaaaaaaaaaaaaaaaa

SEQ ID NO: 20 - DNA, 1926 bp, Artificial Sequence
  Description of Artificial Sequence  Synthetic Primer
ggaaaacataaacataaacatggaactaagtatcatgttatgtttctttacttctattctcttcattgttctgttcagaatattcatcaaatcctttgtctcaaaaagacatgacttgccactcccacctggttcaatgggttggccttacataggagaaacttttcaactttattctcaagaccctaatgtcttctttgcatcaaaaatcaaaaggtatggttctatgttcaagtctcacattttgggatgtccatgtgtgatgatttcaagtcctgaagcagcaaaatttgtgctgaataaagctcaacttttcaagccaacattccctgctagcaaagagaggatgttgggaaaacaagctatcttttttcatcaaggagagtatcatgctaatttaagaagacttgttcttcgcacgttcatgccggtagccatcagaaacattgttcctgacattgaatccattgctgaagatagtcttaaatcaatggaaggacggttaatcaccactttccttgaaatgaaaacgttcacattcaacgttgctctactttcaatttttggaaaagatgaaattcactaccgagagcaattaaaacagtgttactacactctagaaaaagggtacaattcaatgccaattaatcttccaggaacactcttccataaggcaatgaaagctagaaaagaacttgcacagatcctagctcaaataatctcaagtagaagagagaagaaagaagaatacaaagatttgttaggttcattcatggatgaaaaatcaggactaagtgatgaacaaatagcagataatgtaattggagttatttttgcagctcgtgataccacagctagtgtgcttacgtggattgttaagtaccttggtgaaaatatcagtgtcctagaatcagtgattgaggaacaagaatctatattgaagagcaaagaagaaaatggtgaagaaaagggtcttaattgggaagatacaaagaaaatggttataacttcaagggttattcaagagactcttagagttgcttcaattttgtctttcacttttagagaagcagttgaagatgttgaatatcaagggtatcttataccaaaagggtggaaagtattgccactatttaggaatatacatcatagtccaaataacttcaaagatccagaaaagtttgatccttcaagatttgaggctgccacaaaacccaatacttttatgccatttggcagtgggatccacgcttgtcctggcaatgaattagccaagatggagattttagtcctcttacaccatctgaccacaaagtacaggtggtctgtggagggtacaaagaatgggattcaatatggcccttttgctcttccccaaaatggattgcccataacattatatcctaagaagtagataacacttcaacataagattgatcagccacaccatattctatagaatcattttaaatatggaaataatgaattcttatcattgttctcaaaattggctctaactctctcaacaaaatgaggaaagatcaagacaatgttgcaagggagattagatcaactcttgctttctttttggcctatagacacccaaagggtagtcttgagaatagaacagaaatagggatggagtttgtcaagtgtatataagaggtttcatagcaagagcaaatagtttgagtttttattttttatttttttaactttatttttctttccaattttgaggaaaagtcctcaaaatattgtattggtagttgttacttgttacattttatctcacctttgtaacctacaaataattttagtgtccacctaacaaatttcctaaggacatttgttagagacacctgattgaataattttaattactcaaaaaaataagtgttcaatattaaaaaaaaaaaaaaaaaa

SEQ ID NO: 21 - DNA, 1888 bp, Artificial Sequence
  Description of Artificial Sequence  Synthetic Primer
ggcaaccctttgactctggagtaacactaacttcttctttaaagcaaaaaaccatcgccatctccaccatgctattccaatccatcatggattttctatcaaatccttttctttttgcagctttgtctgcatctttaactcttttgttggttcaacttctgctcagaaaattgaacaacaaaagcaataacatgaagaagaaaaagtatcatcctgttgctggcactgtattcaatcagatgatgaatttcaacagacttcatcattatatgactgatcttgcaaggaaatacaggacatacaggctacttaaccctttcagaagtgaagtttatacttcagaaccaagtaatgttgagtatatactcaaaaccaactttgagaactatggaaagggattgtacaactaccaaaatttgaaggatttactaggagatggaattttcgccgttgatggtgagaaatggcgtgaacaaaggaagatatcaagtcatgaattctccacgaagatgttacgggatttcagtacttcaatattcagaaagaatgctgcaaaagttgcaaatatagtgtctgaagctgcaacttctaattttaagttagaaatccaagatcttttaatgaaatcaaccttggattcaattttccaagttgcatttggaactgaacttaacagcatgtgtggatcaagtgaggaaggaaagaacttcgccaatgcttttgatactgcaagtgcgttaacgctttatcgttatgttgatgtcttttggaagataaagaagtttctcaatattggatcagaggcagcattaaggaaaaacactgaagtcttaaatgaatttgtcattaagctaatcaacactagaattcaacaaatgaattcaaagggtgactctattagaaagagtggagatattctatcaaggtttctgcaagtgaaggaatatgatacaacatacttaagagatataattctgaactttgttattgctgggaaagacacaacggccgctacactttcttggttcatgtatatgctatgcaagtatcctgcagtacaagaaaaagctgcagaagaagtgagagaagcaacaaacacaaaaacagttagcagctgcactgagtttgtgtcatgtgtaacagatgaagctcttgaaaagatgaattatctccatgcaacactcacagagactctcagactttatcctgcagttcctgtggatgcaaaaatttgctttgctgatgacacattaccagatggatatagtgtaaaaaaaggagacatggtgtcataccaaccttatgcaatggggaggatgaaattcatatggggtgatgatgcagaggaatttagacctgcaagatggctggatgaaaatggcaattttcaggcagagaaccctttcaagtttactgcttttcaggcaggtcctcggatatgcctaggaaaagagtttgcttatagacagatgaagatattctcagcagttttattaggttgttttcgtttcaaattgaatgatgagaagaggaatgtgacttataagacaatgataaatcttcatattgatggaggacttgaaatcaaagcattacacagggattagaagatgattccgtgcaacaaatcaaactctaattagcaagaagttatgcttattttatatgtgataatggatgactgtattcatcaaacataaaaggtgttattttgtaaggaattgtgctcaaacctttaagcatagctaaaatgtagccctctcgtacataatgatgaactagataaaataagattttgtcaaacattaataatttaaagggaaatttaaagggagcaaatataataatatttgcattgaaaaaaaaaaaaaaaaaaaaaaaaaaaaaa

SEQ ID NO: 22 - DNA, 1704 bp, Artificial Sequence
  Description of Artificial Sequence  Synthetic Primer
aggaaaaaataatatggaagtgtttatgtttcccacaggaacaacaataatcatctctgttctttcagttctacttgctgtgattccatggtatcttctcaacaagttatggcttaaaccaaagaggtttgagaaacttctcaaagctcaaggttttcaaggtgaaccatataacctttcagtatttaaggataaatcaagacaaaattatatgttgaagttgcaacaagaagataaatctaaattcattggtctctccaaagaagctgcaccatctatcttcactcatgttcatcaaactgtacacaaatatggaaaaaaatcctttttatgggaaggtacaacaccaagagttatcatcgcagaccctgatcaaattaaggaagtctttaacaagatcgaggacttccccaaaccaatattaaaatccatcgccaagtatttgagcgttggtataatacattatgagggtaagaaatgggctaaacataggaagatcgccaatccggcattccacctagaaaaattgaaaggtatgctacctgcattttcttacagttgcaatgaaatgattagtaaatggaaggaactattgtcatcagatggaacatgtgaggttgatgtttggcctttccttcagaattttacctgtgatgtaatttctcggacggcatttggaagcagctacgcagaaggagaaaaactatttcaacttctaaagaagcagggatttcttttgatgacagggcgacaaacgaacaatccattatgggggcttctagcaacaactaccaagacgaagatgaaagaaattgatagagaaatccatgattcacttgagggaatcattgaaaagcgagaaaaagcactgaagaatggtgaacccaccaatgacgatttattaggcattcttttgcaatcaaatcatgccgaaaaacaaggacatggaaatagtaagagtaatgggatgaccacccaagatgtgatagatgaatgcaaattgttttacattgctgggcaagagaccacctcaagtttgctggtttggacaatggtgttattaggcaggtatcctgaatggcaagcacgtgcaaggcaggaagttttgcaagtttttgggaaccaaaatccaaacatcgaaggattaaatcaacttaaaattgttaccatgattttgtatgaggtactaaggttattcccacctttaatttacttcaaccgagctcttcgaaaggatttgaaacttggaaacgtttcgctacctgaaggaacacaaatttccctaccaatactattgattcaccaagatcatgatctatggggtgatgatgcaaaggagttcaaacctgaaaggtttgctgaaggaattgcaaaggctacaaaaggaaaagtttcttatttcccttttggatggggtcctagaatttgtcttggacaaaactttgccttactagaagcaaagatagcaatttcattgctgctgcagaatttctcattcgaactttctccaaattatgtgcatgttcccaccactgtgcttactttgacgccaaaaaatggtgcaagcatcattttgcataaactgtaagagcacatccaatggagttattcagtagctttactcttttaggtgatttatctgtaaacatgagtttctttaaattaagacatattgatttgtataagaaaaaaaaaaaaaaaaaa

SEQ ID NO: 23 - DNA, 2006 bp, Artificial Sequence
  Description of Artificial Sequence  Synthetic Primer
ggcctaaaaaaaaaatttgaacatcacattacaggcttacagctctaacaataatttctaacgccaattatggatgtgatgaataatttgttctcactatcaacattgccatttacaatctgcattttgttgcttctctttgttctcttttcgctacgtagaagcaatattactacgggtgcagcttcaatgacaccaccacccgaagctgctggcgcttggcctttaatcggccacctccacctcttaggtggttcccaacaaccttatatcaccttgggaaacttagccgacaaatacggagccatcttcacagtgcgtttaggtgttcatcgaactttagttgttagcagttgggaaattgctagacagtgtttcactgtaaacgacaaagcctttgcttctcgtcccaaatctatagcctttgaaatcatgggttataactcttccatgtttggtatgagtccctacggttcttattggcgtacattgcgtaagatcgccactgtccacgtcctctcagctcaacgaatagatatgcttaaacatgttatggaatctgaggtgaagaaagctatgaaagatagttactcattttggctaaggatgaagaatgatggtaactctgaaagagctattacggaaatgaaaaaatggtttggtgatatagcgatgaacgttatgtgtagaacggtaacggggaaagtttttgatggtgacgaagaagagaatcagaggattagaaaatcctttagggactttttcgatctcagcggttcatttgttatatctgacatgttgcggttttttagatggttggatttggatggaaaacagaagcagatgaagaaaacggctaaagagttagatgattttgttcaagtttggctcgatcaacacaaacgcaacaagaaacctgccggcaccaaacttgacttcatggatgtgctcctttcaaccgttgatgatcaagatatagatggtcgtgatgctgacaccacaatcaaagcaacttgtctggcactaattctagcaggtacagacactaccgcagcgacattgacctggtctgtttctttacttcttaacaatcctgaagttttaaacaaagccattcaagaattagatacacaaattggtatggaaaatatggcaatagaatcagattttgcaaagtttgaatatctcaaagccattatcaaggaaacattgcgtctgtacccagccgcaccactcgatgtgcctcatgagtccattgaagattgtaccgttggtggataccacgtgccagccggtacgcgtctcataactaacctttcgaaacttcaacgagatccaatgttatattcggatccgcatgagtttcgaccagagagattccttacaacgaacaaagatgtcgatgtcaagggccaacattttgagttgattccatttggtgcgggtagaagaatatgtcctggaatctcatttagtcttcagctgatgcaaataacacttgctactttattgcatgggtttgacattgtgactaaagatggaggaccagttgatatggttgaacaaagcggactcaccacaatcaaagcctctccacttgaagtcattcttactccacgtttgtctaccgaagcttttagtcaaaattaatgctctaggttaagtcacaaaattaatcttcaacgttgtgccaattaaatgcaagtacaagcatagttcttgttctccaatttttgctatattgcagaatttgatagtaactttgtggctttggtgtaccgaccttggaaaactttggtgcctacaaagttgcatttgatatgcaagtggctcaatttgtagtttgtaggtatggggtgtgattcagtgaacaatttccttttcttcatttacaagtgtaattaaaaaggaaaataaatgcacgacacatactggacagtttaaaggctttatgaaaagttgacacgtacctattcttgcaaaaaaaaaaaaaaaaaaaaaaaaaa

SEQ ID NO: 24 - DNA, 1964 bp, Artificial Sequence
  Description of Artificial Sequence  Synthetic Primer
gggtggttgtcgaatatccacatagcttagctcaccggacgcaactcttgattgatgtgtctcgggttgcaagttcaaatttaattgatgagattcgaagatgtttttcttttacataaaacacctcattttcatatttgttttaagcctcggaataagttgcgttgattttgactaacactattaattttgaatatacaaatgtaatttgtaagatttcatcatggggtttcttgtgcttctttctctcttgccaatcttaattctattcattatacacatctacaaaataaggagtactagtagagcatcatcaactccaccaggtccaaaaccacttcctctaattggaaatctacaccagcttgacccttcatccccacatcactccttatggaaactttccaaacactatggacctatcatgtctttgcaacttggttacataccaaccttaattgtttcctcagcaaaaatggcagaacaagtgttgaaaacccatgaccttaaatttgcaagtagaccatctttcctaggactaagaaaattgtcttacaatggtttagatcttgcttttgcaccttatagtccttattggagagagatgagaaaactttgtgttcaacatctctttagctctcaacgtgtccattcttttaggcccgttagagaaaatgaagtggcccaattgattcaaaagttgtcgcaatatggtggtgatgaaaaaggtgcgaacttgagtgaaatattgatgtctctcacaaatacaattatatgtaagatagcttttggaaaaacatatgtttgtgattatgaagaagaagttgaattgggaagtggacaaaagagaagtagattgcaagttttgcttaatgaagctcaagctttgttggctgaattttacttttcagataattttccattgttgggttggattgatagagtcaaaggaactcttgggaggcttgataaaacattcaaggagttggatttgatataccaaagagttattgatgaccacatggataattcagcaaggcctaaaactaaggaacaagaagtagatgatattattgatatcttattgcagatgatgaatgatcactcactctcttttgatctcactcttgaccacatcaaagctgtgcttatgaacatttttatagcaggaacagacacaagttcagcaatagtggtttgggctatgacaacattgatgaacaatcctagagtgatgaacaaggttcaaatggaaatcagaaacttatatgaagacaaatattttataaatgaagatgatattgaaaagctaccttatcttaaagcagtggtgaaagagacaatgagattatttccaccatcaccattactagtaccaagagaaacaatagaaaattgtaacatagatggttatgagattaaaccaaaaactttagtgtatgttaatgcatgggccataggaagggatcctgagaattggaaagaccctgaagagttttatcctgaaaggttcattatgagttcagtggactttaaagggaaaaattttgagctaattccatttggaagtggaagaagaatgtgtcctgcaatgaacatgggagtggtcactgttgagcttacacttgctaatcttcttcactcttttgattggaagttgcctcatggttttgacaaggaacaagtgttggatacaaaagtgaaaccaggaataactatgcataagaaaattgatctttgcctttttcctaggaaaagaaaaccataggatatatgtactattgacaattagttatgttatttgcatgcctcaaagtttgtattatttctagatcaattgataatggtttcatttgtaatttgggaagtgattaaaagagacttgtaatatgttttcatcttataatgttaacttgtatttgggggagttatgtacccattgtgataaaaaaaaaaaaaaaaaa

SEQ ID NO: 25 - DNA, 1611 bp, Artificial Sequence
  Description of Artificial Sequence  Synthetic Primer
ggcatccatccatccatccttaactctatctttaaacttttcctatcctctatgggttcaactgagaatgagaatggaaatttgaagcctcttcatgttgcaatgctcccatggcttgctatgggacacgtactcccttattttgagttggccaaaattcttgctcaaaatggtcacactgtcacctttatcaactctcccaaaaacattgatcaaattcctaaaccacccaaaacaattcaaccattcatcaatttggttaaatcacctttaccacatatagaacaactacaaggtgaagagagcatgcagaatattccaaaaaacatgattggttatcttaagttggcttatgacggtctacaagacaatgttactgatatactcaaaacttcaaagcctgattgggttttctatgattgtgcagctgattggttgccggcaattgccaaaagccttaacattccttgtgctcattacagtatactcgcagctttgaacgtatgtttctttaatccacctagggatcaagccgtaaacatgtgtagcccaccaaagtggcttcctttcgaaacaattgtttatctcaaaccttatgagatgatgagaataaaggaatctgttaagaatgagtctggtggaaaaacagtcactactgctgataccagcaaagtattcacaagtgctgacatgtttcttattagaacctctagagaacttgaaggtccatggttagattatctttctcaccgatacaaggttcctgtgcttcctgttggagttcttccaccatccttgcatataagagacgaccaacatgatgaaaacaaccctgattgggtccacatcaaggcatggttggactcaaaagaatcatcttctgttgtttacattggatttggaagcgagtcaaagttagatcaacaagatttaactgagttagctcatggaattgaactttctgggttacctttcttttgggctttgaaagatcgtaaagacggtgtatctgaattacctcaaggattcgaggaaagaacaaaagaacgtggaattgtttggaaaacctgggtaccccagatcaaaatcttagctcatccatcaattggtggatgtatgagtcactgtggtggaagttcagtcgttgagatgcttcatcttgggcatgttcttgtcacattgccttatatacttgaccagtgtttgtatgcaagattactagaagaaaagaaagtggctgttgaagtaccaaggagtgagcaagatgggtcctttactagggactctgtagccaaaacattgaggttggtaatagtggatgaggaaggtagcacgtgcaggaaaaatgctaaagatatgggaaaaattttcagttcaaaagatcttcacaatcaatacattaaagatttaatcgctgctcttcaaaagcatagagttcattccgacagttaagcatatttcatgtgtttccctttcaattttttattttatttttaatcttattgcaataatagtccatgagatgttgatgtcagttcattggcaagagtttgactttgtaatttcatgggacaaagttcaagagtttgatcattgttaccaccaataatg

SEQ ID NO: 26 - DNA, 1651 bp, Artificial Sequence
  Description of Artificial Sequence  Synthetic Primer
ggtagctttgtactctaaccttctcttcatttccaatttctcataatcatcaaaatgaaggatactttagttctttacccagccctaggaaaaggacacctgaactctatgattgagttaggtaaactcatattaacacataacccttcatattccatcacaattcttatcctcaccccaccaaataccaccttgcaaccaccacaagagatccaaaaactcacaacaacaaccacctttggttgtgaatcttttccatctattactttccatcacattcctcctatttcattcccagttacactcccacctcatatagtcccacttgaagtttgtggtcgtagtaaccaccatgttaaccatgttcttcaatccatttcaaaaacctcaaaccttaaaggtgttattttggatttcatgaactatagcacaaaccaaatcacttcaactcttgatataccaacttactttttctacacttcaggagcttcaactcttgctgtttttcttcaacttccaaccattcatcaaagtaccacgaaatcgcttaaagagtttcacatgtatcctagaatccctgggttaccattggttcctatagttgatatgcccgatgaagtgaaggatcgtgagagtaaaagttacaaggttttcttagatatggcgacaagtatgagggaaagtgatggagttatcataaacactttcgatgccattgaaggaagagctgcaaaagctttaaaagcagggttgtgtctaccagaaggaacaacacctccattgttttgtattggaccaatgatttcacctccttgtaagggtgaagatgaaagagggagttcatgtttgagttggctcgactcgcaaccaagtcaaagcgtcgtgttgttgagctttggaagcatgggaagattttctagggctcaattgaatgagatagctattggattggagaaaagtgagcaaagattcttgtgggttgttaggagtgaaccagactcagacaagttgagtttggacgagttatttccagaagggtttttggagaggacaaaggacaagggaatggttgtgagaaattgggccccacaagttgcgatattgagtcataactccgtgggtggatttgtgactcattgtggatggaactccgtgttggaagctatttgtgaaggggtgccaatgattgcatggcctttgttcgcagaacaaaggctaaatagattggttttagtcgatgaaatgaaggtggctttgaaagtgaaccaatcagaaaataggtttgtgagtggcacagagttgggtgagagagttaaggagttgatggaatcggaccgtggaaaggatattaaagagaggattttgaaaatgaaaataagtgctaaggaggcaagaggaggaggtggatcttctcttgttgatttgaaaaagttgggagattcatggagggagcatgcttcttggaatagtttatcaccaaattcccctttccttcttcgttgaaaattaaaaatagatctttagagggaataaaattcgaaaagtgtgtccatgctaataaaaaaaattataaatatgtgtattatattgagatgtgaatcaaaattagtagtatcttttttatgtcatgaaaaaaaaaaaaaaaaaaaaa

SEQ ID NO: 27 - DNA, 1587 bp, Artificial Sequence
  Description of Artificial Sequence  Synthetic Primer
ggaaaatccagaactcacaaattcaccatgactaacgaaaatcaagaacttcacataatcttcttcccatttctagccaatggccacatcataccttgtgtagaccttgcaagagtcttctcttcaagaggactcaaattcaccattgtcacaactcatctcaatgtacctctcatttcaagaaccattggaaaagctaaaatcaacatcaaaactatcaaattcccttcaccagaagaaaccggtttaccggaaggttgcgaaaattctgaatcagcattagcaccagacaagttcatcaagttcatgaaatcaacccttcttctaagggaaccacttgaacatgttttggaacaagaaaaaccagattgtttagttgctgacatgtttttcccttggtcaactgattctgcagcaaaattcaacattcctaggattgtgtttcatggtttaggtttcttccctttatgtgttttggcttgtacaagacagtacaaacctcaagataaagtctcatcttacacagaaccttttgttgttcctaatcttcctggagaaatcacactgacgaagatgcagttaccgcaacttcctcagcatgataaagtcttcacaaaactattggaagagtctaatgaatcagaagtgaaaagctttggtgtgattgcaaacactttctatgaacttgaaccggtttatgctgatcattatcgaaacgagcttggaagaaaagcttggcatttaggtccagtttctttatgcaatagagacactgaagaaaaagcatgtagaggaagagaagcatcgatcgacgaacacgagtgtttgaaatggctacaatcaaaagaaccaaattcagttatttatgtttgttttggtagcatgacggttttcagcgacgctcagcttaaggaaattgcaatgggacttgaagcttctgaagttccattcatttgggttgtgaggaaaagtgctaaaagtgaaggtgaaaatttggaatggctaccagaaggttttgaggaaagaattgaaggtagtggtaaaggattgatcataagaggttgggcaccacaagtgatgatattggatcatgaatcagttggagggtttgtgacacattgtggatggaattcaacattggaaggagtgagtgcagggttaccaatggtgacatggccaatgtatggtgaacaattttacaatgcaaagtttttgagtgatatagttaagattggtgttggtgttggggtgcaaacttggattggaatgggaggtggtgagcctgtgaagaaagatgttatagagaaggcagtgagaaggatcatggttggggatgaagcagaggaaatgagaagcagagcaaaggagtttgggaaaatggctagaagagctgtggaggttggtggatcttcttacaatgattttagcaatttaattgaggatttgaagtcacgtgcatactaatgtgtatgcaattagtggcaatatgtttgttcacatgttgtgtgtttaaagtcgatgacacatatcttatcttagtatcaataaatgttacgaacactcagtttaaaaaaaaaaaaaaaaaaa

SEQ ID NO: 28 - DNA, 1625 bp, Artificial Sequence
  Description of Artificial Sequence  Synthetic Primer
ggactacaactccttcttcttcttcaccctattcatccaaaaacataagtgctccatttgaatccaaaaaaatatcaatccaacagtaatgtctcaagaaaagcaaaataaaatagagtatactcctcatggtcatagccaaaagcctcatgttgtattagcaccatttccagcacaaggtcatgtgaaccctttcatgcaattagccaaactcctacgttgcaacggttttcacataacctttgtgaacactgaattcaaccacaaacgtttgataaaatctcttggagctgagtttgtgaagggtctaccagattttcaatttgagaccatacctgatggtttgccagagtcagataaagatgcaacacaagatattccaacgttgtgtgatgcaactagaaaaaattgttatgctcctttcaaagagcttgtgattaagctcaacacttcatcacctcatattccagttacttgcataattgctgatggtaattatgactttgctggaagagtggctaaagatttgggcattcgagagatacaactttggacagcttctacttgtgggtttgtggcatatttgcaattcgaggagcttgtcaaaagaggaattcttccattcaaagatgaaaattttattgccgatggcaccttggatacaagtttagattggatctctggaataaaagacatcagattgaaagaccttccaagcttcatgagagtcactgatctaaatgatattatgtttgatttcttttgtgttgagccaccaaattgtgtgagatcatcagcaatcatcattaacacatttgaagaattagaaggtgaagccctggacacccttagggccaaaaaccctaacatatatagcattggcccacttcacatgcttggtaggcattttcctgagaaagaaaacggttttgcagcaagtggttcaagtttttggaaaaatgactctgaatgcataaaatggttgagtaaatgggaacctggctcagtactatatattaattacggaagtataactgttatgacagatcatcacttgaaagaatttgcttggggaatagcaaatagcaaattaccatttttgtggattatgagaccagatgtagtaatgggtgaagagacttcatctttgcctcaagagtttctagatgaagttaaggatagaggatacataactagttggtgctatcaagatcaagtgctttctcatccatcagttgggggattcttgactcattgtggttggaattctacacttgaaactatttcctatggtgtgcctactatttgttggcctttctttgctgagcaacaaacaaattgtaggtatttatgcaacacttggaaaatagggatggaaattaactatgatgtgaaaagagaagagataagagaacttgtgatggaaatgatggaaggagaaaaaggaaaagaaatgagacaaaagagtttggtgtggaagaagaaagctacagatgctactaatttgggaggatcatcatacattaatttctataatttaattaaagagcttcttcatcacaatgctatttgagttatattataatcggtctattacttttagttaaaaaaaaaaaaaaaaaaaa

SEQ ID NO: 29 - DNA, 1752 bp, Artificial Sequence
  Description of Artificial Sequence  Synthetic Primer
ggaattatttcccctcaaaaaaaattcagcagcaatggaaggtgttgaagtcgaacggcctttgaaacttcacatgcttccatttctatcacctggtcatatgattcctttgggtgacatagcagctctgtttgcatcccatggccaacaagtcacgatcatcaccactccctccaatgctcatttctttaccaaatctctctcctctgtcgatccgttcttcctccgccttcacaccatcgacttcccctcccagcaagtcgacctctccgacggagttgaatcattgtcctccaccgatgaccctgccaccatggccaagatatgcaaaggtgcaatgctcctccatgaacccattagagaatttgtggagaaggatcaacctgactacatcattgccgactgtgtatacccttggattaatgacttgaccaataagcctcatatctccaccattgccttcaccggatactctctctttacagtatcccttatagaatccctaagaataaaccgttcttatcctggcaagaattcaagttcgagttcgttcgttgttccagactttcctcattctatcaccttttgctcaacaccaccaaagatattcatcgcatatgaggaaaggatgcttgagacaatccgtaaaagtaagggactcatcattaacagctttgctgaacttgatggtgaagattgcatcaaataccatgagaaaaccatgggttataaggcttggcatcttggtccagctagtcttattcgcaaaacttttgaagagaaatccatgaggggaaatgagattgtggttagtgcccaagagtgtctaagttggctcaattcaaaggaagaaaattcagtgttatacatatgttttgggagtatctcttatttctctgataaacaactttatgagattgcgagcggaatagaaaattcaggtcacgaatttgtatgggttgttcctgagaagaaggggaaagaagatgagagtgaagaggagaaagaaaagtggttgccaaaaggattcgaagagagaaatattggaaataagaaaggttttatcattagggggtgggccccacaagtaatgattttaagccacactgttgtgggcgcattcatgacacattgcgggtggaactccaccgctgaggcggttagtgcagggattccgatgattacgtggccagtgcgaggagaacaattctataatgaaaaactcataagtgttgtgcgagggattggggtggaggttggtgcatcagagtgggctctacatggttttcaagaaaaagagaaagtggtgagtagacatagtatagaaaaagctgtgaggagattgatggacgatggtgatgaagcaaaggaaatcagacgacgtgctcaagagtttgggagaaaggctgcacaagctgttcaagaaggcgggtcttctcataacaatttgttgactttgattgacgatcttcaaagattgagagaccgcaaaccacttgaataattcaaatcttattattatgtatattcaactaatttgaaacccatccccgcattgaaaatttgtgttgaatgttatatatatatatatatatatatatatatatatatattaatttatgttgataatatttgttgcaaaaataaatagtacatgtcaaatgtattatatcttatatcttatcttatgtataataaagggaatacacatatctttgagtgatttcttttacaaaaaaaaaaaaaaaaaaa

SEQ ID NO: 30 - DNA, 1726 bp, Artificial Sequence
  Description of Artificial Sequence  Synthetic Primer
ggcaaacataacatcaaaagatagaattatcaaaaatgggaaactttgcaaacagaaaaccacatgttgtgatgattccatatccagttcaaggccatatcaatccattgttcaaactagcaaagcttcttcaccttagaggctttcacataacctttgttaacaccgaatacaatcacaaacgcttgctcaaatcaagaggtccaaaggcttttgatggtttcacggactttaactttgagagcattccagatggtttaacaccaatggaaggtgatggtgatgttagtcaagatgtaccaactctttgtcaatcagtaagaaagaacttccttaaaccctattgtgaacttcttacaagacttaaccactctaccaatgttccaccagttacttgcttagtttctgattgttgtatgagctttactatacaagctgctgaagaatttgaactcccaaatgttctctatttttcatcaagtgcatgttctttattgaatgttatgcactttcgttcctttgtagaaagaggtatcataccattcaaagatgagagttatctaacaaatggatgtttggaaactaaagtagattggattcccggtttgaaaaactttcggttgaaggacatcgttgactttatcaggacaacaaatccaaatgatattatgttagaattctttatagaagttgcagatcgagttaacaaagacactactattcttttgaatactttcaatgaacttgagagtgatgtaataaatgctctctcctccacaattccttctatttatccgattggccctttaccttcattattaaaacaaactccacaaattcatcaattggactctttagattccaacctttggaaagaagatacagagtgtcttgattggcttgaatccaaggagccgggatcagttgtttatgtgaatttcggcagcattacagttatgacacccgagcaattactggaatttgcttggggtttggccaattgcaagaaatcatttttgtggatcataaggcctgatcttgtcattggtggctcagtgattttctcatctgagtttacaaatgaaattgcagatagaggcctaatagcaagttggtgtccacaagacaaagtgttgaaccacccttcaatcggaggattcttgactcattgcggatggaattcaaccactgaaagtatatgcgctggagtgccaatgttgtgttggccattttttgccgaccagccaacagactgtagatttatttgtaatgaatgggagattggtatggaaatcgatacgaatgtgaagagagaggagttggcaaagctgatcaatgaagtgatagccggagataaaggaaagaaaatgaagcaaaaggccatggagttgaagaagaaggcagaggagaacactagaccaggtggttgttcatacatgaacttgaacaaagttattaaggatgtgttgcttaaacaaaattaagccaggaggtcgtatcgaaatttttaggccattactatgctttgatgtactgtatttttaactatgtattgtttcaatatttaatttgggtttattggtggattattggtaagtgaccaatcgtcaatcaaagtaataaattttaagttcatattcagagtttatcttagattgcattgcataaaagagtaatcaacgattacatggttcagctgaaaaaaaaaaaaaaaaaaaaaaaaaaa

SEQ ID NO: 31 - DNA, 1684 bp, Artificial Sequence
  Description of Artificial Sequence  Synthetic Primer
gggaaagaacaaagaaaatgtctatgagtgatataaacaagaattcagaactcatcttcattcctgcaccaggaattggccacttagcttcagctcttgaatttgcaaaacttttaaccaaccatgacaaaaatctttacatcacagtcttctgcatcaagtttccaggcatgccctttgcagattcatatatcaaatcagttttagcctcacaaccacaaattcaactgattgatcttcctgaagtagaaccacctccacaagagctactaaaatctccagaattttacatcttgacttttttggagagtctcatacctcatgtcaaagcaactatcaaaaccattttatcaaacaaagttgttgggttagtcctagatttcttttgtgtttcaatgattgatgttggaaatgaatttggtatcccttcttatttgtttctaacatcaaatgttggttttttaagtctcatgctttcccttaaaaaccgccaaatcgaagaagttttcgatgattccgaccgtgatcatcagttgttgaatattcctggtatctcaaaccaagttccttctaatgttttacctgatgcttgttttaataaagatggtggatatattgcttattataaactagctgagaggtttagagacaccaaagggattattgttaataccttttcagatttggaacaatcttctattgatgcattatatgatcatgatgagaaaatccctcctatctatgctgttggtcctttgttagatctcaaaggtcagcctaaccctaaattggatcaagctcagcatgatcttatattgaaatggctagatgagcagccagataaatcagttgtttttttatgttttggaagcatgggagttagctttggtccatctcaaataagagagatagcattaggacttaagcatagtggggttaggttcttgtggtctaacagtgcagagaaaaaagtgttcccagaagggtttttagaatggatggaattggaaggtaagggaatgatatgtggatgggcaccacaagttgaggttttggcacataaggctattggtggatttgtttcacattgtggatggaattctattttggaaagtatgtggtttggtgtaccaatattgacatggcctatttatgcagaacaacagcttaatgcttttaggttggtgaaggaatggggggtaggtttgggactgagagtggactatagaaagggtagtgatgttgtagcggccgaggagattgagaaaggattgaaggatttgatggataaagatagcattgtacacaagaaggttcaagagatgaaagagatgtctaggaatgctgttgttgatggtggatcttctttaatttctgttggaaaacttattgatgatattacaggaagcaactgataaactgtctttttttgctacataggtggagtttccctttcttggaatcaatggatgaagaagacattctatatgttatattgttttgttgagggatgtcattttatatactatattctacctaaaaaactgttgaaagaataaaagttgaatgtggaattagtagcatatttgtgtatagcaaatttaatcaagctagcacatgtgcctatctttttttatttcagtactgcttttctttggagggttgtttatataatattttttta